SEQUENCE LISTING (25 sequences)

SEQ ID NO: 1 - DNA, 40 bp, Artificial
  Primer
gcttacctgaccgacctctgcctatcccctgttgcgtgtc

SEQ ID NO: 2 - DNA, 40 bp, Artificial
  Primer
ccattccccagctcgtcttgccatctgttccctccctgtc

SEQ ID NO: 3 - DNA, 20 bp, Artificial
  Primer
gcttacctgaccgacctctg

SEQ ID NO: 4 - DNA, 20 bp, Artificial
  Primer
ccattccccagctcgtcttg

SEQ ID NO: 5 - DNA, 44 bp, Artificial
  Primer
ccattccccagctcgtcttgccatctgttccctccctgtctcag

SEQ ID NO: 6 - DNA, 20 bp, Artificial
  Primer
ccatctgttccctccctgtc

SEQ ID NO: 7 - DNA, 20 bp, Artificial
  Primer
cctatcccctgttgcgtgtc

SEQ ID NO: 8 - DNA, 20 bp, Artificial
  Primer
cgtttcccctgtgtgccttg

SEQ ID NO: 9 - DNA, 20 bp, Artificial
  Primer
ccatctgttgcgtgcgtgtc

SEQ ID NO: 10 - DNA, 20 bp, Artificial
  Primer
ccatctgttccctccctgtc

SEQ ID NO: 11 - DNA, 20 bp, Artificial
  Primer
cctatcccctgttgcgtgtc

SEQ ID NO: 12 - DNA, 51 bp, Artificial
  Primer
tattgttgatgctgtaaaaagaagctactggtgtagtatttttatgaagtt

SEQ ID NO: 13 - DNA, 47 bp, Artificial
  Primer
tgctcaaagaattcatttaaaatatgaccatatttcattgtatcttt

SEQ ID NO: 14 - DNA, 48 bp, Artificial
  Primer
aagcgaacagtcaagtaccacagtcagttgacttttacacaagcggat

SEQ ID NO: 15 - DNA, 47 bp, Artificial
  Primer
tacaggtgttggtatgccatttgcgatttgttgcgcttggttagccg

SEQ ID NO: 16 - DNA, 52 bp, Artificial
  Primer
aacatataaacatcccctatctcaatttccgcttccatgtaacaaaaaaagc

SEQ ID NO: 17 - DNA, 39 bp, Artificial
  Primer
tagatatcacttgcgtgttactggtaatgcaggcatgag

SEQ ID NO: 18 - DNA, 41 bp, Artificial
  Primer
attcaactctggaaatgctttcttgatacgcctcgatgatg

SEQ ID NO: 19 - DNA, 40 bp, Artificial
  Primer
gatgaggagctgcaatggcaatgggttaaaggcatcatcg

SEQ ID NO: 20 - DNA, 45 bp, Artificial
  Primer
tgtatctcgatttggattagttgctttttgcatcttcattagacc

SEQ ID NO: 21 - DNA, 40 bp, Artificial
  Primer
cattaacatctgcaccagaaatagcttctaatacgattgc

SEQ ID NO: 22 - DNA, 46 bp, Artificial
  Primer
gcgacgacgtccagctaataacgctgcacctaaggctaatgataat

SEQ ID NO: 23 - DNA, 43 bp, Artificial
  Primer
aaaccatgcagatgctaacaaagctcaagcattaccagaaact

SEQ ID NO: 24 - DNA, 44 bp, Artificial
  Primer
tgttgctgcatcataatttaatactacatcatttaattctttgg

SEQ ID NO: 25 - DNA, 51 bp, Artificial
  Primer
gcagatggtgtgactaaccaagttggtcaaaatgccctaaatacaaaagat

(19) United States
(12) Patent Application Publication
(10) Pub. No.: US 2005/0221341 A1
(43) Pub. Date: Oct. 6, 2005

(54) Sequence-based karyotyping

(76) Inventors: Richard A. Shimkets, Guilford, CT (US); Michael S. Braverman, New Haven, CT (US)

Correspondence Address: MINTZ LEVIN COHN FERRIS GLOVSKY & POPEO, 666 THIRD AVENUE, NEW YORK, NY, 10017, US

(21) Appl. No.: 10/971,614

(22) Filed: Oct. 22, 2004

Related U.S. Application Data
(60) Provisional application No. 60/513,691, filed on Oct. 22, 2003.
(60) Provisional application No. 60/513,319, filed on Oct. 23, 2003.

(51) Int. Cl. C12Q 1/68; G06F 19/00; G01N 33/48; G01N 33/50

(52) U.S. Cl. 435/6; 702/20

(57) ABSTRACT

A new method for genomic analysis, termed “Sequence-Based Karyotyping,” is described. Sequence-Based Karyotyping methods for the detection of genomic abnormalities, for diagnosis of hereditary disease, or for diagnosis of spontaneous genomic mutations are also described.

### RELATED APPLICATIONS

This application claims the benefit of priority from U.S. Application Nos. 60/513,691 and 60/513,319, both filed Oct. 22, 2003. All patents and patent applications referenced in this specification are hereby incorporated by reference herein in their entireties.

### FIELD OF THE INVENTION

The invention relates to the field of genetics. In particular, it relates to the determination of karyotypes of genomes of individuals cells and organisms.

### BACKGROUND OF THE INVENTION

Structural rearrangements of chromosomes have played a decisive role in the development of abnormalities in animals. It is also known that inversions, translocations, fusions, fissions, heterochromatin variations and other chromosomal changes occur as transient somatic or hereditary mutation events in natural populations. In human cancer, chromosomal changes, including deletion of tumor suppressor genes and amplification of oncogenes, are hallmarks of neoplasia (1). Single copy changes in specific chromosomes or smaller regions can result in a number of developmental disorders, including Down, Prader Willi, Angelman, and cri du chat syndromes (2). Current methods for analysis of cellular genetic content include comparative genomic hybridization (CGH) (3), representational difference analysis (4), spectral karyotyping/M-FISH (5, 6), microarrays (7-10), and traditional cytogenetics. Such techniques have aided in the identification of genetic aberrations in human malignancies and other diseases (11-14). However, methods employing metaphase chromosomes have a limited mapping resolution (about 20 Mb) (15) and therefore cannot be used to detect smaller alterations. Recent implementation of comparative genomic hybridization to microarrays containing genomic or transcript DNA sequences provide improved resolution, but are currently limited by the number of sequences that can be assessed (16) or by the difficulty of detecting certain alterations (9). There is a continuing need in the art for methods of analyzing and comparing genomes.

Traditional karyotyping is usually performed on lymphocytes and amniocytes using labor intensive methods such as Giemsa staining (G-banding). Because chromosomes are visualized on an optical microscope, the ability to resolve detailed mutations (involving only a small part of a chromosome) is limited. While more detailed karyotyping techniques, such as FISH (fluorescent in situ hybridization) are available, they rely on specific probes and it is not economically or technically feasible to perform FISH on the entire chromosome set (i.e., the complete genome).

In recent work, a method was provided for karyotyping a genome of a test eukaryotic cell by generating a population of sequence tags after restriction endonuclease digestion from defined portions of the genome of a test cell (17). This method is not optimal because a small number of areas of the genome are expected to have a lower density of restriction endonuclease cleavage sites and could be incompletely evaluated. The authors estimate these areas to encompass 5% of a genome. Furthermore, the resolution of the method is dependent on the restriction enzyme used and the method cannot reliably detect very small regions of the genome on the order of several thousand base pairs or less.

Very recently, a new type of human polymorphism in genomic DNA has been described, in which small gene regions are repeated or deleted (18). These changes, known as Copy Number Polymorphisms (CNPs), may account for a variety of human disease conditions. New methods of analysis will be needed to identify these polymorphisms and thereby detect a wide variety of human or animal diseases or the traits of any eukaryotic organism including humans, non-human animals and plants.

### BRIEF SUMMARY OF THE INVENTION

The current invention provides for a method of karyotyping a genome of a test cell (e.g., eukaryotic or prokaryotic) by generating a pool of fragments of genomic DNA by a random fragmentation method, determining the DNA sequence of at least 20 base pairs of each fragment, mapping the fragments to the genomic scaffold of the organism, and comparing the distribution of the fragments relative to a reference genome or relative to the distribution expected by chance. The number of a plurality of sequences mapping within a given window in the population is compared to the number of said plurality of sequences expected to have been sampled within that window or to the number determined to be present in a karyotypically normal genome of the species of the cell. A difference in the number of the plurality of sequences within the window present in the population from the number calculated to be present in the genome of the cell indicates a karyotypic abnormality.

Other embodiments, objects, aspects, features, and advantages of the invention will be apparent from the accompanying description and claims.

The present invention provides for a method of karyotyping a genome. The genome of the cell is karyotyped by randomly fragmenting the DNA from a cell and sequencing at least a portion of each fragment. Optimally, at least 20 base pairs of each fragment is sequenced. For example, the DNA is fragmented by an enzyme that cleaves DNA. The enzyme cleaves at specific locations within the DNA. Alternatively, the enzyme cleaves the DNA randomly, i.e., non-specifically. For example the enzyme is DNase. The DNA is cleaved by mechanical method such as sonication or nebulization. The DNA is sequenced by methods know in the art.

Preferably, the test cell and the reference cell is from the same species. The cell is a eukaryotic cell or a prokaryotic cell. The eukaryotic cell a mammalian cell. The mammal is, e.g., a human, non-human primate, mouse, rat, dog, cat, horse, or cow. The cell is a cancer cell, an embryonic cell, or a fetal cell. The cell is isolated from amniotic fluid or is derived from in vitro fertilization. Optionally, the cell is from a subject with a hereditary disorder.

The plurality of DNA sequences obtained are mapped to a genomic scaffold to create a distribution of mapped sequence to a region of the genome. At least 1000, 10,000, 100,000, 1,000,000 or more sequenced are mapped. The sequences map to one or more regions in the genome. The regions are on the same chromosome. Alternatively, the regions are on different chromosomes. The distribution are within a contiguous region of the genome. Alternatively, the distributions are within discontiguous regions of the genome, e.g., on different chromosomes.

By mapping to a genomic scaffold is meant that the sequences are aligned along each chromosome. The test cell distribution (i.e., chromosomal map density) is defined as the number of mapped sequences (i.e., fragments) by the number of possible map locations present in a given chromosome. The number of possible map locations is defined by the size of the observation window and the length of the chromosome. No particular length is implied by the term observation window. For example, the observation window is 25 Mb, 10 Mb, 5 Mb, 4 Mb, 2 Mb, 500 kb, 250 kb, 60 kb, 30 kb, or 10 kb or less in length.

The test distribution is compared to a reference distribution from a reference cell and an alteration between the test distribution and the reference distribution is identified. The reference distribution can be a database of mapped sequences from previously tested cells. Identification of an alteration indicates a karyotypic difference between the test cell and the reference cell. The alteration is statistically significant. By statistically significant is meant that the alteration is greater than what might be expected to happen by change alone. Statistical significance is determined by method known in the art. An alteration is statistically significant if the p-value is at least 0.05. The p-values is a measure of probability that a difference between groups during an experiment happened by chance. (P(z≧z_{observed})). For example, a p-value of 0.01 means that there is a 1 in 100 chance the result occurred by chance. The lower the p-value, the more likely it is that the difference between groups is caused by a karyotypic difference. Preferably, the p-value is 0.04, 0.03, 0.02, 0.01, 0.005, 0.001 or less. Alternatively, the p-value is 1/24, 1/23 or 1/22 or less.

The method of the invention is useful in detecting aneuploidy. For example, aneuploidy is detected when the test distribution to reference distribution is greater than 1.5 or less than 0.75. However, if the test region and reference region is in a sex chromosome and the cells are from a subject of the opposite sex. aneuploidy is detected when the test distribution to reference region distribution is greater than 3.0 or less than 1.5.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1. Chromosome Content computed using Sequence-Based Karyotyping data is highly correlated with previously published estimates using the Digital Karyotyping method. Each point represents a chromosome, with extreme values representing an extra (>3.0) or the loss (<1.5) of a whole chromosome.

FIG. 2. 4 Mb resolution fragment density maps identifying regions of amplification and deletion. Amplification on chromosome 7. Center panel represents Sequence-Based Karyotyping 4 Mb density map as compared to the approximately 4 Mb published maps (inset, top right).

FIG. 3. 4 Mb resolution fragment density maps identifying regions of amplification and deletion. Chromosomal content across chromosome 2. Center panel represents Sequence-Based Karyotyping 4 Mb density map as compared to the approximately 4 Mb published maps (inset, top right).

FIG. 4A. Schematic depicting the methods of the invention and various embodiments for these methods.

FIG. 4B. Schematic depicting exemplary therapeutic and diagnostic applications for the disclosed methods, including infectious disease, oncology, inflammation, and disease diagnostics.

FIG. 5. Schematic depicting exemplary fields for use of the disclosed methods, including agriculture and industry, drugs and diagnostics, bio-defense and public health, and academia and government.

FIG. 6. Schematic depicting an overview of sample preparation for the disclosed sequencing methods.

FIG. 7. Schematic depicting an overview of Parallel Sequencing™.

FIG. 8. Schematic depicting a comparison method used for whole-genome sequencing.

FIG. 9. Schematic depicting an overview of Sequence-Based Karyotyping.

FIG. 10. Schematic depicting an overview of sequence-based gene expression analysis.

FIG. 11. Schematic depicting an overview of genome-wide methylation analysis.

FIG. 12. Schematic depicting an approach for complex-sample sequencing.

FIG. 13A. Schematic depicting the first and second steps for the cell population sequencing method.

FIG. 13B. Schematic depicting the third through seventh step for the cell population sequencing method.

FIG. 14 Schematic representation of the universal adaptor design according to the present invention. Each universal adaptor is generated from two complementary ssDNA oligonucleotides that are designed to contain a 20 bp nucleotide sequence for PCR priming, a 20 bp nucleotide sequence for sequence priming and a unique 4 bp discriminating sequence comprised of a non-repeating nucleotide sequence (i.e., ACGT, CAGT, etc.). FIG. 14 depicts a representative universal adaptor sequence pair for use with the invention. FIG. 14 depicts a schematic representation of universal adaptor design for use with the invention.

FIG. 15 Depicts the strand displacement and extension of nicked double-stranded DNA fragments according to the present invention. Following the ligation of universal adaptors generated from synthetic oligonucleotides, double-stranded DNA fragments will be generated that contain two nicked regions following T4 DNA ligase treatment (FIG. 15). The addition of a strand displacing enzyme (i.e., Bst DNA polymerase I) will bind nicks (FIG. 15), strand displace the nicked strand and complete nucleotide extension of the strand (FIG. 15) to produce non-nicked double-stranded DNA fragments (FIG. 15).

FIG. 16 Schematic of one embodiment of a bead emulsion amplification process.

FIG. 17 Schematic of an enrichment process to remove beads that do not have any DNA attached thereto.

FIG. 18 Depicts an insert flanked by PCR primers and sequencing primers.

FIG. 19 Depicts the calculation for primer candidates based on melting temperature.

FIG. 20 Depicts the assembly for the nebulizer used for the methods of the invention. A tube cap was placed over the top of the nebulizer (FIG. 20) and the cap was secured with a nebulizer clamp assembly (FIG. 20). The bottom of the nebulizer was attached to the nitrogen supply (FIG. 20) and the entire device was wrapped in parafilm (FIG. 20).

FIGS. 21A-F Depict an exemplary double ended sequencing process.

FIG. 22 Depiction of jig used to hold tubes on the stir plate below vertical syringe pump. The jig was modified to hold three sets of bead emulsion amplification reaction mixtures. The syringe was loaded with the PCR reaction mixture and beads.

FIG. 23 Depiction of beads (see arrows) suspended in individual microreactors according to the methods of the invention.

FIG. 24 Depicts a schematic representation of a preferred method of double stranded sequencing.

FIG. 25 Illustrates the results of sequencing a Staphylococcus aureus genome.

FIG. 26 Illustrates the average read lengths in one experiment involving double ended sequencing.

FIG. 27 Illustrates the number of wells for each genome span in a double ended sequencing experiment.

FIG. 28 Illustrates a typical output and alignment string from a double ended sequencing procedure. Sequences shown in order, from top to bottom: SEQ ID NO: 12-SEQ ID NO:25.

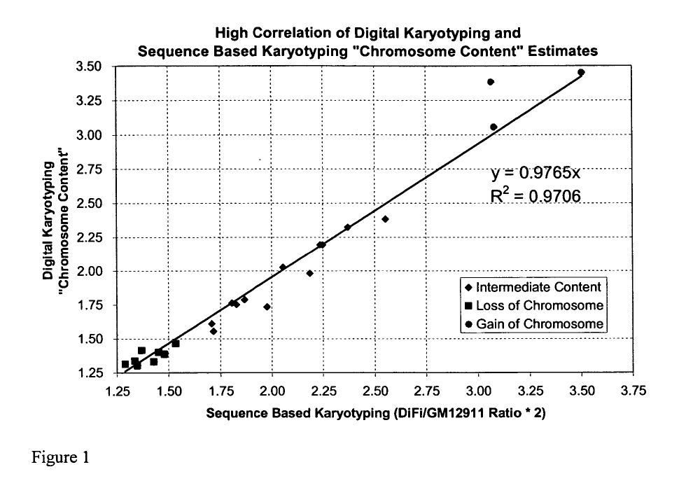

For FIGS. 1, 2, and 3, graph values on the Y-axis indicate genome copies per haploid genome, and values on the X-axis represent position along chromosome.

### DETAILED DESCRIPTION OF THE INVENTION

The term “karyotype” refers to the genomic characteristics of an individual cell or cell line of a given species; e.g., as defined by both the number and morphology of the chromosomes. Typically, the karyotype is presented as a systematized array of prophase or metaphase (or otherwise condensed) chromosomes from a photomicrograph or computer-generated image. Alternatively, interphase chromosomes may be examined as histone-depleted DNA fibers released from interphase cell nuclei. In one embodiment, the karyotyping methods of this invention are also used to determine Copy Number Polymorphisms in a test cell or a test genome. Since the Sequence-Based Karyotyping methods may be performed on prokaryotic cells, the presence of chromosomes is not essential for the methods of the invention.

As used herein, “chromosomal aberration” or “chromosome abnormality” refers to a deviation between the structure of the subject chromosome or karyotype and a normal (i.e., “non-aberrant”) homologous chromosome or karyotype. The terms “normal” or “non-aberrant,” when referring to chromosomes or karyotypes, refer to the predominate karyotype or banding pattern found in healthy individuals of a particular species and gender. Chromosome abnormalities can be numerical or structural in nature, and include aneuploidy, polyploidy, inversion, translocation, deletion, duplication, and the like. Chromosome abnormalities may be correlated with the presence of a pathological condition (e.g., trisomy 21 in Down syndrome, chromosome 5p deletion in the cri-du-chat syndrome, and a wide variety of unbalanced chromosomal rearrangements leading to dysmorphology and mental impairment) or with a predisposition to developing a pathological condition. Chromosome abnormality also refers to genomic abnormality for the purposes of this disclosure where the test organism (e.g., prokaryotic cell) may not have a classically defined chromosome. Furthermore, chromosome abnormality includes any sort of genetic abnormality including those that are not normally visible on a traditional karyotype using optical microscopes, traditional staining, of FISH. One advantage of the present invention is that chromosomal abnormality previously undetectable by optical methods (e.g., abnormalities involving 4 Mb, 600 kb, 200 kb, 40 kb or smaller) can be detected.

As used herein, the term “universal adaptor” refers to two complementary and annealed oligonucleotides that are designed to contain a nucleotide sequence for PCR priming and a nucleotide sequence for sequence priming. Optionally, the universal adaptor may further include a unique discriminating key sequence comprised of a non-repeating nucleotide sequence (i.e., ACGT, CAGT, etc.). A set of universal adaptors comprises two unique and distinct double-stranded sequences that can be ligated to the ends of double-stranded DNA. Therefore, the same universal adaptor or different universal adaptors can be ligated to either end of the DNA molecule. When comprised in a larger DNA molecule that is single stranded or when present as an oligonucleotide, the universal adaptor may be referred to as a single stranded universal adaptor.

“Target DNA” shall mean a DNA whose sequence is to be determined by the methods and apparatus of the invention. These include a test genome or a reference genome.

Binding pair shall mean a pair of molecules that interact by means of specific non-covalent interactions that depend on the three-dimensional structures of the molecules involved. Typical pairs of specific binding partners include antigen-antibody, hapten-antibody, hormone-receptor, nucleic acid strand-complementary nucleic acid strand, substrate-enzyme, substrate analog-enzyme, inhibitor-enzyme, carbohydrate-lectin, biotin-avidin, and virus-cellular receptor.

As used herein, the term “discriminating key sequence” refers to a sequence consisting of at least one of each of the four deoxyribonucleotides (i.e., A, C, G, T). The same discriminating sequence can be used for an entire library of DNA fragments. Alternatively, different discriminating key sequences can be used to track libraries of DNA fragments derived from different organisms.

As used herein, the term “plurality of molecules” refers to DNA isolated from the same source, whereby different organisms may be prepared separately by the same method. In one embodiment, the plurality of DNA samples is derived from large segments of DNA, whole genome DNA, cDNA, viral DNA or from reverse transcripts of viral RNA. This DNA may be derived from any source, including mammal (i.e., human, nonhuman primate, rodent or canine), plant, bird, reptile, fish, fungus, bacteria or virus.

As used herein, the term “library” refers to a subset of smaller sized DNA species generated from a single DNA template, either segmented or whole genome.

As used herein, the term “unique”, as in “unique PCR priming regions” refers to a sequence that does not exist or exists at an extremely low copy level within the DNA molecules to be amplified or sequenced.

As used herein, the term “compatible” refers to an end of double stranded DNA to which an adaptor molecule may be attached (i.e., blunt end or cohesive end).

As used herein, the term “fragmenting” refers to a process by which a larger molecule of DNA is converted into smaller pieces of DNA.

As used herein, “large template DNA” would be DNA of more than 25 kb, preferably more than 500 kb, more preferably more than 1 MB, and most preferably 5 MB or larger.

It is a discovery of the present inventors that the genome of an organism can be sampled by random fragmentation and sample sequencing to determine karyotypic properties of a cell, tissue, or organism using a systematic and quantitative method. The method of the invention can be used to determine changes in copy number for portions of the genome on a genomic scale. Such changes include gain or loss of whole chromosomes or chromosome arms, interstitial amplifications or deletions, as well as insertions of foreign DNA. Rearrangements, such as translocations and inversions, can be detected by the method of the invention, e.g., where large fragments are generated and the ends sequenced, or where the scaffold-predicted ends are a different distance apart than the size of the fragment sampled.

The data shown herein demonstrate that the method of the invention, called Sequence-Based Karyotyping, can accurately identify regions whose copy number is abnormal, even in complex genomes such as the human genome. Advantageously, the method permits the identification of specific amplifications and deletions that had not been previously described by comparative genomic hybridization (CGH) or other methods in any human cancer. The approach is particularly applicable to the analysis of human cancers, wherein identification of homozygous deletions and amplifications has historically revealed genes important in tumor initiation and progression. The method of the invention can be used with a variety of other applications. For example, the approach could be used to identify previously undiscovered alterations in hereditary disorders. A potentially large number of such diseases are thought to be due to deletions or duplications too small to be detected by conventional approaches. These may be detected with Sequence-Based Karyotyping, even in the absence of any linkage or other positional information.

The methods of the invention may be used for diagnosis of diseases, or a propensity to develop diseases. For example, Chronic Myeloproliferative Diseases (MPD) are associated with one or more of the following abnormalities: +14 or trisomy 14, +8 or trisomy 8, −21 or monosomy 21, -Y, del (13q), del(16)(q22), del(20q), del(5q), and del(9q). Myelodysplastic Syndromes (MDS) are associated with one or more of the following abnormalities: +11, trisomy 11, +14, trisomy 14, +15, trisomy 15, +8, trisomy 8, −21, monosomy 21, −7/del(7q), −7/del(7q), -Y, del (13q), del(13q), del(16)(q22), del(17p), del(20q), del(5q), and del(9q). Acute Non Lymphocytic Leukaemias (ANLL) are associated with one or more of the following abnormalities: +10, trisomy 10, +11, trisomy 11, +14, trisomy 14, +15, trisomy 15, +22, trisomy 22, +4, trisomy 4, +8, trisomy 8, −21, monosomy 21, −7/del(7q), -Y, del (13q), del(16(q22), del(17p), del(20q), del(5q), and del(9q). B-Cell Acute Lymphocytic Leukaemias (B-ALL) are associated with one or more of the following abnormalities: +10; trisomy 10; +15; trisomy 15; +4; trisomy 4; +8, trisomy 8; −21, monosomy 21; Trisomy 5 and del(6q). T-Cell Acute Lymphocytic Leukaemias (T-ALL) are associated with one or more of the following abnormalities: +4, trisomy 4, +8, trisomy 8, del(6q); and del(9q). Non Hodgkin Lymphomas (NHL) are associated with one or more of the following abnormalities: +12, trisomy 12, +3, trisomy 3, +8, trisomy 8, del (13q), del(11q), del(13q), del(17p), del(6q) and del(7q). Chronic Lymphoproliferative Diseases (CLD)) are associated with one or more of the following abnormalities: +12, trisomy 12, +15, trisomy 15, +8, trisomy 8, −21, monosomy 21, del (13q), del (6q) and del(13q).

The methods of the invention may be used to determine chromosomal abnormalities including balanced and unbalanced chromosomal rearrangements, polyploidy, aneuploidy, deletions, duplications, copy number polymorphisms and the like. The chromosome abnormalities that are detectable by the methods of the invention include constitutional or acquired abnormalities. Numeric abnormalities that are detectable include polyploidy (e.g., tripolidy or tetraploidy) or aneuploidy (e.g., trisomy, monosomy). Other abnormalities that can be detected by the methods of the invention include abnormalities of chromosome structure such as translocations (balanced or unbalanced), deletions, inversions (e.g., pericentric inversion and paracentric inversion), duplication, or isochromosomes. The structural anomalies such as translocations and inversions may be in the balanced or unbalanced forms.

Standard chromosome analysis (e.g., G-banding) allows only the detection of only relatively large structural rearrangements while more detailed analysis rely fluorescence in situ hybridization (FISH) technology that require specific molecular probes. FISH probes for small chromosomal abnormalities may involve the actual gene or a critical region surrounding the genes. Current technology is still unable to detect certain microdeletions and microduplications.

One embodiment of the invention is directed to a method of karyotyping a test genome of a test cell. The first step in Sequence-Based Karyotyping is to obtaining a plurality of test DNA sequences from random locations of the genome of the test cell. DNA is isolated from a test cell to produce a test DNA (or a test genome) using standard methods. In a preferred embodiment of the invention, test DNA sequence is determined by randomly fragmenting the test DNA into multiple fragments and sequencing at least 20 basepairs from each fragment. Randomly fragmenting a DNA refers to the physical fragmentation (e.g., also called breakage or digestion) of a large molecule of DNA into multiple smaller DNA molecules in a non-sequence specific manner. The non-sequence specific fragmentation (random fragmentation) is distinguished from sequence specific fragmentation which may involve, for example, restriction endonuclease digestion. In other words, non-sequence specific fragmentation (random fragmentation) may involve a method of fragmenting DNA without the use of restriction endonucleases.

One method of randomly fragmenting a nucleic acid is to use enzymatic digestion or physical fragmentation. Enzymatic digestion of DNA may involve digestion of DNA with a DNA cutting enzyme such as DNase I, endonuclease V or the like which does not exhibit sequence specificity. Physical fragmentation may involve sonication or nebulization. In addition, DNA fragments may be generated by random PCR amplification (i.e., PCR with random primers). Additional methods for preparing DNA fragments may be found in copending U.S. application Ser. No. 10/767,894 filed Jan. 28, 2004, incorporated herein by reference in its entirety.

After fragmentation of the test DNA, a portion or all of the fragments may be sequenced for at least 20 contiguous bases. The sequencing of more than 20 bp is also contemplated but not necessary. Sequencing may be performed on any part of the DNA fragment such as from the ends or from a region between the two ends of the DNA fragment.

In an optional step, the DNA fragment may be amplified before sequencing. Methods for amplifying DNA are known and are described, in the Examples and in copending U.S. application Ser. No. 10/767,779 filed Jan. 28, 2004 and U.S. application No. 10/767,899 filed Jan. 28, 2004, both incorporated herein by reference in their entireties.

Methods for sequencing DNA fragments are well known. There are many DNA sequencing methods available, such as the Sanger sequencing using dideoxy termination and denaturing gel electrophoresis (Sanger, F. et al., Proc.Natl.Acad.Sci. U.S.A. 75, 5463-5467 (1977)), Maxam-Gilbert sequencing using chemical cleavage and denaturing gel electrophoresis (Maxam, A. M. & Gilbert, W. Proc Natl Acad Sci USA 74, 560-564 (1977)), pyro-sequencing detection pyrophosphate (PPi) released during the DNA polymerase reaction (Ronaghi, M. et al., Science 281, 363, 365 (1998)), and sequencing by hybridization (SBH) using oligonucleotides (Lysov, I. et al., Dokl Akad Nauk SSSR 303, 1508-1511 (1988); Bains W. & Smith G. C. J. Theor Biol 135, 303-307(1988); Drnanac, R. et al., Genomics 4, 114-128 (1989); Khrapko, K. R. et al., FEBS Lett 256. 118-122 (1989); Pevzner P. A. J Biomol Struct Dyn 7, 63-73 (1989); Southern, E. M. et al., Genomics 13, 1008-1017 (1992)). Other sequencing methods are described in copending U.S. patent application Ser. No. 10/768,729 filed Jan. 28, 2004, incorporated herein by reference in its entirety. It is understood that other methods of sequencing may involve optional steps such as the ligation of sequencing primers to the ends of the fragments or the labeling of the fragments.

While the sequencing of 20 bp from each fragment is sufficient, sequencing of more bases is also useful. For example, the sequencing of at least 25 bp, at least 30 bp, at least 35 bp, at least 40 bp, at least 45 bp, at least 50 bp, at least 55 bp, at least 60 bp, at least 65 bp, at least 70 bp, at least 75 bp, at least 80 bp, at least 95 bp, at least 100 bp have been performed by the methods of the invention and found to be useful but not essential. The sequencing of longer sequences is especially useful for larger genomes (test DNA) or for genomes (test DNA) with extensive repetitive sequences. In addition, we have found that it is not essential for the sequencing to begin at the end of the fragment. Sequencing more than 20 bases from one end may mean, for example, sequencing from base 5 to base 25, sequencing from base 10 to base 35 or sequencing from base 50 to base 72. In one preferred embodiment, sequencing may be performed on both ends of a fragment by double ended sequencing—a technique described in this disclosure. Double ended sequencing will allow two different pieces of sequence information to be determined per fragment and can be useful, for example, in identifying chromosomal translocation points. For example, if one end of a fragment maps to chromosome 7 and the other end maps to chromosome 2, the fragment will indicate a chromosome 7 chromosome 2 translocation. Alternatively, if two ends of a short fragment maps to two distant location on the same chromosome, it will indicate the occurrence of a deletion.

The second step involves mapping the test DNA sequences to a genomic scaffold to obtain a test distribution of mapped sequences to a test region of the genomic scaffold to generate a test distribution of mapped sequences. The identification of at least 20 contiguous bases from a fragment from the previous step will typically allow the mapping of the fragment to a unique location in a genomic scaffold. Briefly, the frequency of a random DNA sequence may be expressed as 4ₙ, where n is the length. A 20 base fragment would be expected to occur only once in a trillion or more bases. Hence, a random 20 base sequence is highly likely to map uniquely on a genomic scaffold such as a human genome with 3.2 billion bases. The location may be expressed, for example, as a number. The human genome comprises 3.2 billion bases and a location may be expressed as a number between one and 3.2 billion. Since the method of the invention involves determining multiple sequences, a plurality of locations (called a test distribution or reference distribution of mapped sequences) for the many fragments may be determined. At this time, the genome of 221 organisms, including humans, are known (see, hypertexttransferprotocol://worldwideweb.genomesonline.org). A further 523 prokaryotic genomes and 453 eukaryotic genome is being completed (Id.). The ability to find the location of a 20 base sequence (or any length sequence as listed in this disclosure) determined by the methods of the invention will increase with time.

A genomic scaffold may be a complete DNA sequence of an organism (e.g., a human) or a smaller portion or fraction thereof. One advantage of the invention is that it is not necessary for a complete genome of a test cell to be karyotyped. Instead, in some embodiments, only a small fraction, the test region, may be selected for analysis. The test region may range in size from a complete genome, to a chromosome, to a chromosome arm, or to a fraction of a chromosome arm. A fraction of a chromosome arm may include, a contiguous regions about 4 Mb, 2 Mb, 500 kb, 250 kb, 60 kb, 30 kb, or 10 kb in length. One benefit of selecting a test region smaller than the whole genome is improved processing time. After a test region is determined, DNA sequence data which falls outside the test region may be discarded or ignored. For example, if the test region only comprise chromosome 7 in human, any DNA sequence which lies outside chromosome 7 can be discarded.

One method of producing a test distribution is to note the location of a plurality of DNA sequences from random locations in a test genome. The mapped DNA sequences can be ordered along each test region (e.g., chromosome), and average test cell distribution (chromosomal map density) defined as the number of mapped sequences (fragments) by the number of possible map locations present in a given chromosome. Each map location may comprise a range of bases such as, for example, 1 kb, 10 kb, 20 kb, 50 kb, 100 kb, 200 kb, 500 kb, or 1 Mb of contiguous sequence. As a further example, a 1 Mb stretch of genomic sequence may be fragmented into 10 map locations of 100 kb each (0-100, 101-200, 201-300, 301-400, 401-500, 501-600, 601-700, 701-800, 801-900, 901-1000). Any fragments which maps to the same range of bases (e.g., 603 kb, 650 kb , 675 kb ) would be considered to be mapped to the same location. The size of the map locations may be varied depending on the resolution required. For example, for a lower resolution karyotype, each map location may comprise 4 Mb to 50 Mb contiguous bases. For a higher resolution karyotype, each map location may comprise 5 kb to 100 kb, 5 kb to 200 kb, 10 kb to 100 kb or 10 kb to 200 kb. When a test genome is fragmented and a plurality of fragments is sequenced, a “test distribution” comprising the location and number of fragment that mapped to that location (frequency) of each location can be produced using the methods of the invention.

A reference distribution is produced by applying the same method used to produce the test distribution with the exception that the DNA molecule that is subjected to Sequence-Based Karyotyping is from a reference cell. In a preferred embodiment, the karyotype of the reference cell is known. In another preferred embodiment, the karyotype of the reference cell is normal (i.e., euploid). In other embodiments, the reference cell has a karyotype that is typical of a well known karyotype abnormality such as trisomy 21. Since male cells (XY) contain a different complement of chromosomes than female cells (XX), a reference cell and a reference distribution can be male or female. When the test region is on an autosome, it is not important whether the test cell or the reference cell is of the same sex. When the test region is a sex chromosome, the differences in sex chromosomes numbers between male and female cells should be taken into account.

It is not necessary to generate a reference distribution by experimental methods. As an alternative, a reference distribution may be calculated from a genomic sequence. Because the random fragmentation method is expected to produce an even reference distribution, the reference distribution may be a corresponding test region of a genome with each location of the region having an equal number of mapped sequences. For example, if 10,000 fragments were mapped to a test region with 10 locations of equal size, each location is expected to have a frequency of 1000 mapped fragments. Some non-uniformness will be introduced by the fact that genomes contain regions of repetitive sequence which are non-uniformly distributed throughout the genome. However, since the genomic reference sequence is assumed to be known, the distribution of these repetitive regions can be pre-calculated and factored in to the reference distribution. Finally, inherent in the sequencing process itself may be a slight bias in favor of sequences with certain compositional characteristics (such as higher or lower GC content, the percentage of nucleotides in a given stretch that are G or C). This bias could be ascertained by calibration experiments and then factored in to subsequent computationally derived reference distributions.

In the third step, the test distribution of mapped sequences and the reference distribution of mapped sequences are then compared to determine a sequence-based karyotype of the test cell. If the test cell and the reference cell have the same distribution of mapped sequences, then the test cell and reference cell would have the same karyotype. Similarly, if the test distribution and reference distribution are different, then the test cell and reference cell would have a different karyotype.

The fourth step of the method evaluates if the differences identified by the third step is a significant alterations (significant difference). In a preferred embodiment, the significant alterations are a statistically significant alteration. The statistical significance of any variation between the test distribution and reference distribution may be calculated by the methods disclosed in the Examples. A significant alteration may have a confidence value (p-value) of less than 0.05, less than 0.01, less than 0.001, less than 1/22, less than 1/23, less than 1/24.

In this assay, the test and reference distribution of mapped sequence should be within a contiguous region in the reference genome. In a preferred embodiment, the contiguous region is within one chromosome. In a more preferred embodiment, the contiguous region is within one arm of a chromosome. In the most preferred embodiments, the contiguous regions is less than or equal to a specific size of DNA. The size may be, for example, 4 Mb, 2 Mb, 500 kb, 250 kb, 60 kb, 30 kb, or 10 kb.

In another embodiment of the invention, the reference and test distribution of mapped sequences comprises more than 1000 members (i.e., 1000 mapped sequences). The number of members may be greater than, for example, 2,000, 3,000, 5,000, 10,000, 20,000, 50,000, 100,000, 300,000, 1,000,000 or 10,000,000.

The Sequence-Based Karyotyping method of the invention may be used to analyze both prokaryotic and eukaryotic cells. Eukaryotic cells may be a cell from any eukaryotic organism including, for example, primate cells, human cells, and cells of livestock. In a preferred embodiment, the test cell and reference cell is from the same species. Both normal and abnormal cells may be a test cell or a reference cell. An abnormal cell may be, for example, a cancer cell, a cell from an individual with a disorder, or a cell infected with another organism (e.g., a virus).

One embodiment of the invention is a method of performing a sequence-based karyotype on a cancer cell or a diseased cell. Numerous diseases states have been associated with an abnormal karyotype (see, e.g., discussion of disease related karyotypes above). Sequence-Based Karyotyping may be performed on a cell suspected of being in a preneoplastic or neoplastic state. Any karyotypic abnormalities, or absence of abnormalities, would be useful in diagnosis.

In another embodiment of the invention, the test cell may be from a person with a hereditary disorder or may be used to diagnose a hereditary disorder. In another embodiment of the invention, the Sequence-Based Karyotyping methods of the invention may be used for prenatal diagnosis. Prenatal diagnosis may involve Sequence-Based Karyotyping of a naturally fertilized or in vitro fertilized embryo or fetus. The Sequence-Based Karyotyping methods of the invention may be used for in vitro diagnosis of fetuses based on a sample from amniotic fluid collection procedure or from a chorionic villus sampling procedure.

In one embodiment, the Sequence-Based Karyotyping methods of the invention may be used to determine aneuploidy or copy number polymorphisms. It is understood that the discussion in the specification regarding the detection of aneuploidy is also applicable to the detection of copy number polymorphisms. For example, if one or more autosomes are present in the test eukaryotic cell relative to the reference eukaryotic cell at a ratio of 1.5 or greater or less than 0.75 wherein such ratio is indicative of aneuploidy. A ratio of 1.5 or more (i.e., test/reference>=1.5) is indicative of the presence of at least one extra copy of the autosome or fragment of autosome in the test genome relative to the reference genome. Alternatively, a ratio of 0.75 or less (i.e., test/reference<=0.75) indicates that there may be one less copy of the autosome in the test genome relative to the reference genome.

In another embodiment, the Sequence-Based Karyotyping methods of the invention may be used to determine aneuploidy in sex chromosomes (i.e., X and Y chromosomes). If the test cell and reference cell are both male or both female, then the test is similar to the situation of the autosomes above. In the case where the reference cell is male, the test cell is female, and the test region is on the X chromosome, a ratio of 3 or more (i.e., test/reference>=3) is indicative of the presence of at least one extra copy while a ratio of 1.5 or less (i.e., test/reference<=1.5) indicates that there may be one less copy of the sex chromosome in the test genome.

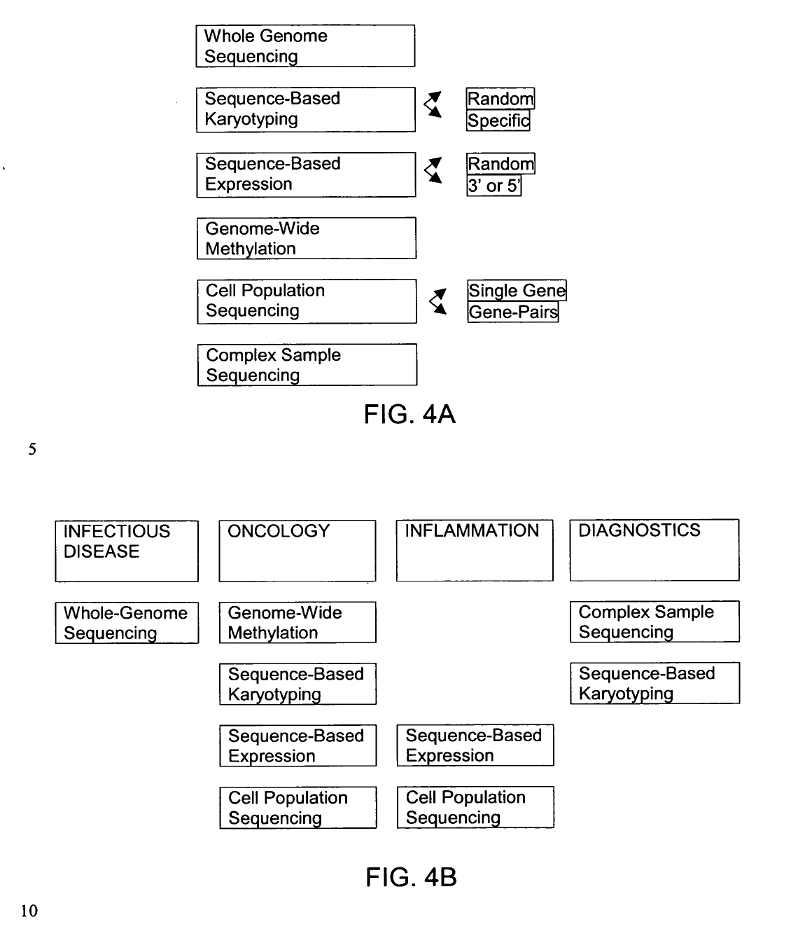

The methods of the invention (e.g., whole-genome sequencing, Sequence-Based Karyotyping, sequence-based expression analysis, genome-wide methylation analysis, cell population sequencing, and complex sample sequencing) encompass various embodiments (FIG. 4A). For example, Sequence-Based Karyotyping can be performed on random or specific samples. Sequence-based expression analysis can be performed on random or 3′ or 5′ samples. Cell population sequencing can be performed on single genes or gene pairs.

In a method of the invention, Genomic DNA of a cell is fragmented and the sequence of the DNA is determined. DNA is fragmented by chemical or mechanical means. The DNA sequences obtained are mapped to a genomic scaffold. By mapping to a genomic scaffold it is meant that the sequences are aligned along each chromosome. Filtering is performed to remove DNA sequences within repeated regions and to remove the rare DNA sequences not present in the human genome. The filtered, mapped DNA sequences are ordered along each chromosome, and the average test cell distribution (chromosomal map density), defined as the ratio of the number of mapped sequences (fragments) to the number of possible map locations present in a given region, is evaluated.

The methods of the invention are useful for many different therapeutic and diagnostic applications (FIG. 4B). As non-limiting examples, the disclosed methods can be used for large-scale sequencing efforts relating to infectious disease. In oncology, the disclosed methods can be used for tumor immunotherapy and improved quality and value of targets for last remaining oncogenes. In inflammation, the disclosed methods can be used for improved target quality and breakthroughs in understanding and treatment of immune disorders. In diagnostics, the disclosed methods can be used in diagnostics platforms and discovery of markers for commercialization on other platforms: protein markers, RNA markers, SNPs, repeats, methylation sites. The methods address the continuing need for testing and treatments for pathogenic infections. The methods are also useful for testing fertilized embryos.

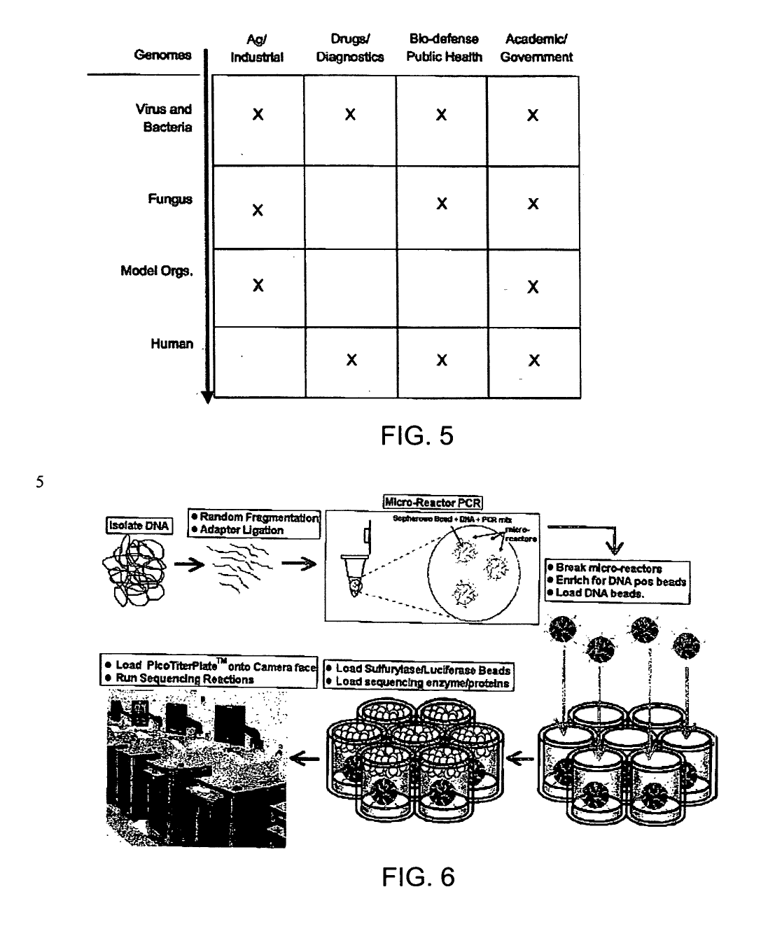

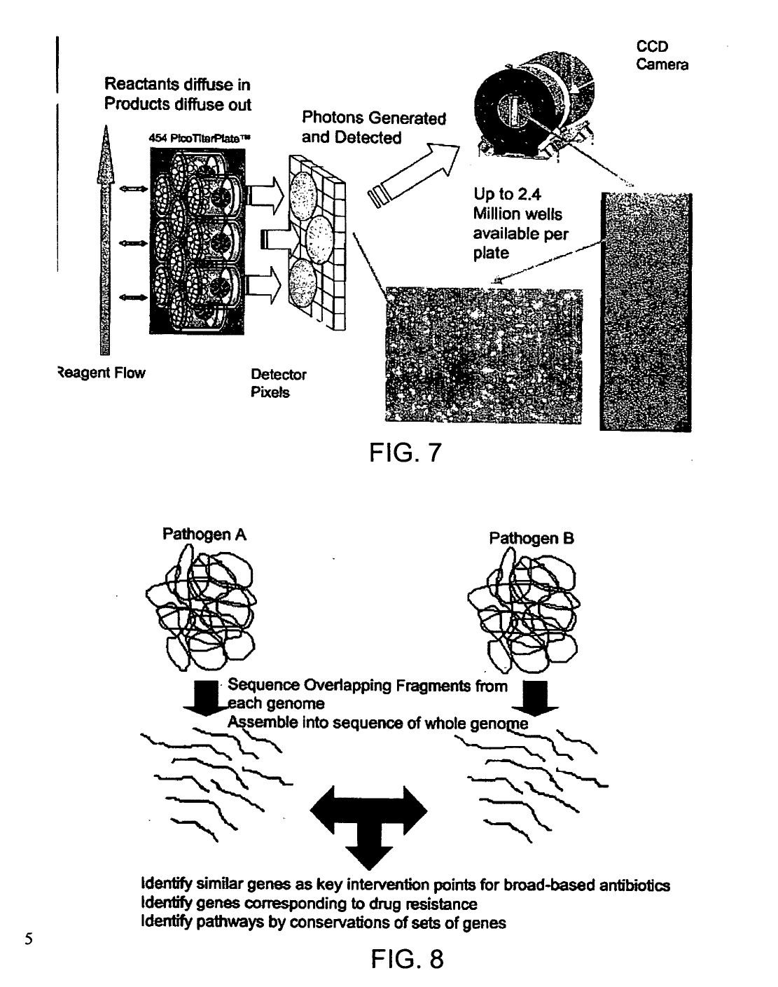

The disclosed methods (e.g., whole-genome sequencing, Sequence-Based Karyotyping, sequence-based expression analysis, genome-wide methylation analysis, cell population sequencing, and complex sample sequencing) can be used in various fields (FIG. 5), including agricultural, industrial, pharmaceutical, diagnostic, bio-defense, public health, academic, and governmental settings. The methods can be applied to a range of genomes such as viral, bacterial, fungal, human genomes, or genomes of model organisms such as worms, flies, zebra fish, chickens, mice, rats, and non-human primates.

The whole-genome sequencing methods of the invention can be used to determine the complete nucleotide sequence of an organism, e.g., for use in virology, infectious disease, human genetics, or diagnostics. These sequencing methods can also be used to identify pathways that use conserved sets of genes. In one embodiment of this method, genomic DNA from two pathogens can be isolated and overlapping fragments can be sequenced (FIG. 8). Based on this, the genome sequence can be assembled (FIG. 8). Whole-genome sequencing can be used to identify common gene sequences among multiple pathogens to locate ideal drug targets (e.g., key intervention points for broad-based drugs such as antibiotics). Sequencing of drug-resistant pathogens allows development of new and tailored therapies (FIG. 8). Non-limiting examples of pathogenic infections include Lyme disease, West Nile virus, HIV/AIDS, tuberculosis, bovine spongiform encephalopathy (mad cow disease), SARS, hepatitis (e.g., hepatitis A and B), influenza, typhoid fever, malaria, cholera, typhoid fever, diphtheria, tick-borne encephalitis, Japanese encephalitis, plague, dengue fever, schistosomiasis, and E. coli infection (e.g., diarrhea). The whole-genome sequencing methods of the invention can be used to study diseases spread by person-to-person contact (e.g., hepatitis B, HIV/AIDS, SARS, tuberculosis, and diphtheria), diseases carried by insects (e.g., dengue fever, malaria, plague, encephalitis, Lyme disease, and West Nile virus), and diseases carried in food or water (e.g., cholera, hepatitis A, schistosomiasis, typhoid fever, E. coli poisoning, and bovine spongiform encephalopathy). Another use of the karyotyping methods of the invention is for the determination of DNA sequence differences between different but related microorganisms. For example, determining differences among the different strains of HIV or influenza, or between different bacteria such a Staphylococcus aureus, can be achieved by sequencing large numbers of DNA fragments derived from each organism, mapping those sequences to a reference genome or directly comparing them to fragments derived from another organism, and identifying differences.

The sequenced-based karyotyping methods of the invention offer a number of advantages over the currently available methods. One advantage is that the present method fragments DNA in a manner that is not sequence specific (i.e., also referred to as random fragmentation). Other methods of DNA fragmentation using, for example, restriction endonucleases are limited in resolution because a small number of areas of the genome are expected to have a lower density of mapping enzyme restriction sites and would be less susceptible to analysis. By some estimates, the percentage of the genome resistant to karyotyping by restriction endonuclease may be as high as 5% (see, e.g., Wang et al.). Since the present methods are restriction endonuclease independent, they can achieve higher resolution than restriction endonuclease dependent methods. In fact, the methods of the invention are limited in resolution only by the number of fragments an operator wishes to sequence, rather than a systematic limitation imposed by the method of sequence fragmentation.

A second advantage of the present method is that the DNA fragmentation technique is not sensitive to DNA methylation. Techniques that employ restriction endonucleases (i.e., Not I) are susceptible to methylation changes in the genome or restriction/protection changes (e.g., in a pathogenic bacteria) and cannot be employed, for example, for the detection of the presence of pathogenic bacterial DNA in a sample of genomic DNA. This is because pathogenic bacteria may comprise a genome that is completely methylated or protected and resistant to restriction endonuclease cleavage. Such a genome would not be detectable by a restriction endonuclease based karyotyping method.

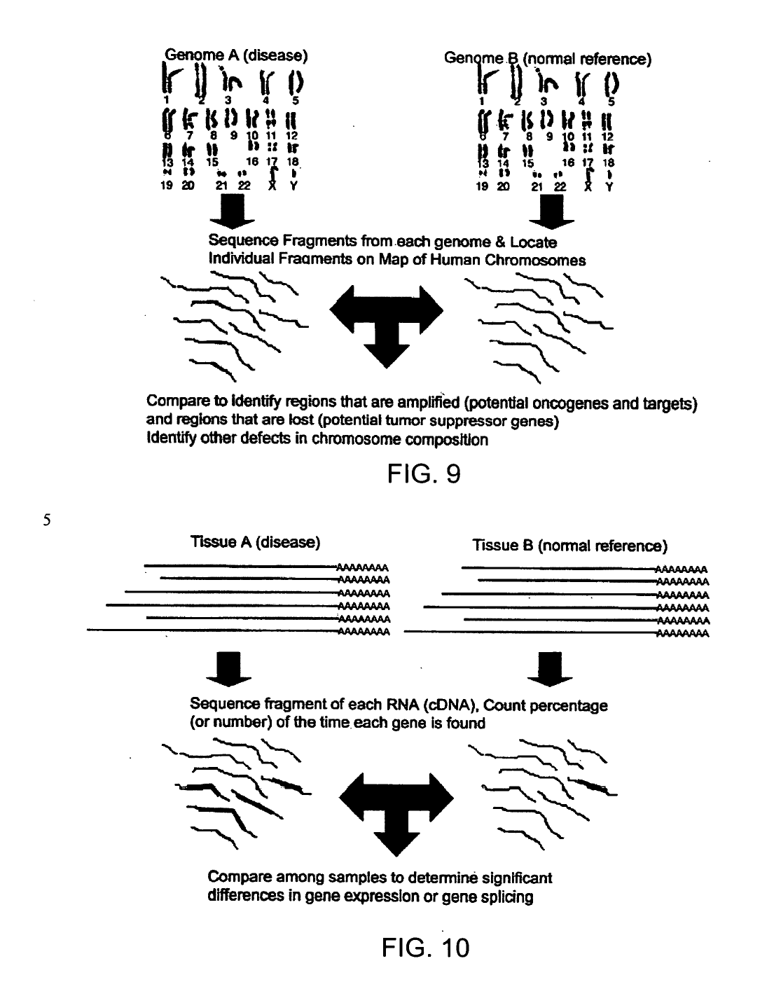

Sequence-Based Karyotyping or high resolution molecular karyotyping according to the invention can be used to identify remaining oncogenes and tumor suppressor genes, or to allow re-implantation diagnostics (e.g., at the single cell level). Such methods can be applied to cancer diagnostics and therapeutics. In one embodiment of this technique, the genomes from a normal subject and a diseased subject are isolated and fragments from each genome are sequenced (FIG. 9). The fragments are located to a map of human chromosomes and the normal and diseased sequences are compared to identify amplifications, deletions, and other abnormalities (FIG. 9). In human cancers, key genes are known to be inserted, amplified, or deleted. The Sequence-Based Karyotyping of the invention can thereby be used to analyze cancer-associated genes and proteins and develop drug targets. The disclosed methods can be used to prepare new and more accurate cancer diagnostics. Sequence-Based Karyotyping can also be used to study diseases (e.g., CNS diseases) of unknown origin. The disclosed methods can also be used to screen in vitro fertilized embryos before implantation. In this way, Sequence-Based Karyotyping can be used to select the healthiest embryos for implantation. This, in turn, can increase the rate of successful implantation over current rates (˜30%).

Another use of the methods of the invention is for the measurement of gene expression in samples. By sequencing a large number of DNA fragments derived from mRNA or cDNA from a given cell or tissue, determining the genes which are expressed in that tissue and at what relative abundance is possible. In addition, applying this method to multiple samples will allow for the comparison among samples in order to identify differentially-expressed transcripts. This method is similar, in principle, to Serial Analysis of Gene Expression (SAGE) except that SAGE samples only the last 10-14 nucleotides of a transcript and thus does not identify variations in splicing, variations in nucleotide sequence relative to a reference genome, and does not always provide a unique identification of the gene based on the small amount of information, all of which is accomplished by gene expression profiling using the sequencing method described in this disclosure. In one embodiment of this method, polyA⁺ RNA is isolated from diseased and normal tissue (FIG. 10). The RNA is reverse transcribed to produce cDNA and this is sequenced. Based on the sequence information, the percentage or number of hits for a particular polyA⁺ RNA is determined (FIG. 10). The diseased and normal samples are compared to identify differences in gene expression and/or gene splicing (FIG. 10). The disclosed sequence-based gene expression methods can be applied, for example, to target identification, toxicology, diagnosis, adverse drug response, determination of drug method of action, drug response, biomarker discovery, co-expression and pathway identification, mutation analysis, and RNAi analysis.

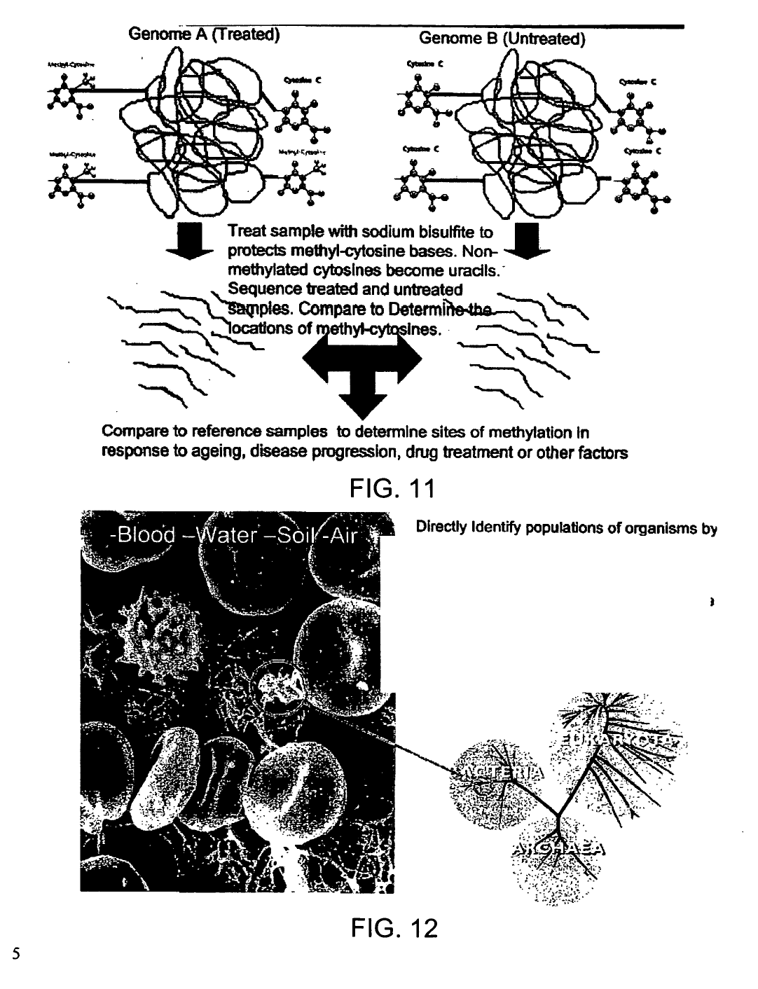

Another use of the sequencing methods of the invention is for the measurement of methylation of DNA. In this method, DNA fragments generated from genomic DNA are sequenced with and without treatment by sodium bisulfite, which modifies unmethylated but not methylated cytosine residues, or another agent that specifically alters either methylated or unmethylated cytosines (FIG. 11). Sequencing a large number of these fragments and comparing them with the genomic reference sequence will determine which nucleotides were methylated. Enrichment of the DNA fragments containing methylated DNA prior to sequencing by the use of a methylcytosine-specific antibody, for example, will make the number of fragments to be sequenced significantly smaller (FIG. 11). Previous studies have correlated methylation patterns with disease progression and drug treatment. Genome-wide methylation studies can therefore be applied to geriatric diseases, drug targets, diagnostics, biomarkers, and forensics. In other aspects, genome-wide methylation analysis can be used to study imprinting.

Complex sample sequencing in accordance with the invention can be used for detection of pathogens in blood, water, air, soil, food, and for identification of all organisms in a sample without any prior knowledge. In accordance with this method, populations of organisms can be identified by preparing a mixed DNA and cDNA sample, sequencing random fragments from the DNA and RNA in the sample, and mapping sequences to a hierarchical database of all known sequences (FIG. 12). According to one embodiment, a cell-free sample (e.g., blood, water, air, food, or soil) can be used to generate 1 million sequence reads. BLAST analysis can be used to assign sequences to known genomes for pathogens. The pathogens can be organized into an evolutionary tree to indicate known agents and/or new agents or strains (e.g., virus or bacteria). Advantageously, this method can be used to identify unknown pathogenic agents and other microorganisms. Complex sample sequencing can also be used for emerging pathogen detection (e.g., by sampling the initial patient set) and for identifying new and useful microorganisms (e.g., in food, water, air, and soil) for medical and industrial applications. This sequencing method can further be used for difficult diagnostic cases, such as the detection of M tuberculosis.

The cell population sequencing methods of the invention can be used to sequence the same gene or pairs of genes (e.g., V_{H}, and V_{L} regions) from 100,00 or more cells. Such studies are ideal for analysis of autoimmunity and tumor immune responses. The cell of interest can be bacterial, fungal, or animal. For example, yeast cells can be analyzed with interacting bait and prey to perform genome-wide pathway studies. Alternatively, B or T cells can be analyzed for variable regions of the immunoglobulin heavy and light chains. Other cells of interest include CD4⁺ cells, CD8⁺ cells, natural killer cells (e.g., tumor infiltrates), and CTLs. Cell population sequencing can be applied to the study of autoimmunity, tumor immunity (e.g., finding common antibodies, cancer mutations), gene mutations (e.g., for oncogenes or tumor suppressors), loss of heterozygocity, protein-protein interactions, and system biology. The methods can thereby be used to identify disease targets and treatments. Cells with interacting pairs of proteins (e.g., bacterial, fungal, or mammalian) can be sequenced to determine pairs of interacting proteins. One embodiment of this method is described as follows.

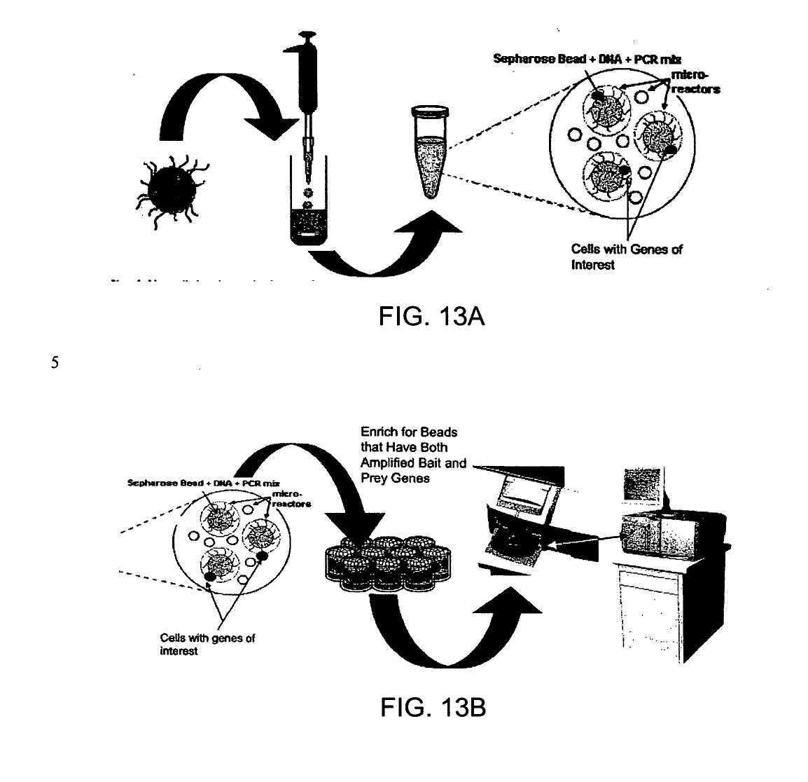

First, magnetic beads are covalently coated with streptavidin and then bound to biotinylated oligonucleotides designed to capture two or more genes of interest from a single cell (FIG. 13A). Second, an aqueous mixture comprising hundreds of thousands to millions of microreactors are generated by mixing together the components for PCR, primer-bound beads, the cell population of interest, and an oil/detergent mixture to create a microemulsion. The aqueous compartments (solid circles in the oil; FIG. 13A) include an average of less than one cell and less than one bead. Third, the microemulsion is temperature-cycled, e.g., in a conventional PCR machine, such that the bead bound oligonucleotides can act as primers for amplification for cells having the target genes (FIG. 13B). Fourth, the emulsion is broken and the beads comprising the amplified genes of interest are isolated, e.g., by magnet. Fifth, after denaturation, the bead are incubated with oligonucleotides that serve as primers for the genes of interest, while at least one primer is added in a de-activated form. Sixth, sequencing is performed on the beads to determine the first sequence of interest. Seventh, the next primer is activated and sequencing is performed on the next gene, e.g., a member of a gene pair (FIG. 13B). Primers can be added sequentially to sequence additional genes captured by this method (i.e., three or more genes).

### 1. Preparing DNA for Sequence-Based Karyotyping

One preferred method for preparing genomic DNA for Sequence-Based Karyotyping is described below. The method is comprised of seven general steps: (a) fragmenting large template DNA or whole genomic DNA samples to generate a plurality of digested DNA fragments; (b) creating compatible ends on the plurality of digested DNA samples; (c) ligating a set of universal adaptor sequences onto the ends of fragmented DNA molecules to make a plurality of adaptor-ligated DNA molecules, wherein each universal adaptor sequence has a known and unique base sequence comprising a common PCR primer sequence, a common sequencing primer sequence and a discriminating four base key sequence and wherein one adaptor is attached to biotin; (d) separating and isolating the plurality of ligated DNA fragments; (e) removing any portion of the plurality of ligated DNA fragments; (f) nick repair and strand extension of the plurality of ligated DNA fragments; (g) attaching each of the ligated DNA fragments to a solid support; and (h) isolating populations comprising single-stranded adaptor-ligated DNA fragments for which there is a unique adaptor at each end (i.e., providing directionality).

The following discussion summarizes the basic steps involved in the methods of the invention. The steps are recited in a specific order, however, as would be known by one of skill in the art, the order of the steps may be manipulated to achieve the same result. Such manipulations are contemplated by the inventors. Further, some steps may be minimized as would also be known by one of skill in the art.

Fragmentation

In the practice of the methods of the present invention, the fragmentation of the DNA sample can be done by any means known to those of ordinary skill in the art. Preferably, the fragmenting is performed by enzymatic or mechanical means. Further, it is preferred that the fragmenting is performed in a non-sequence specific manner. That is, for example, the fragmenting is performed without the use of sequence specific endonucleases such as restriction endonucleases. The mechanical means for fragmentation may be sonication or pnysical shearing. The enzymatic means may be performed by digestion with nucleases (e.g., Deoxyribonuclease I (DNase I)). In a preferred embodiment, the fragmentation results in ends for which the sequence is not known.

In a preferred embodiment, the enzymatic means is DNase I. DNase I is a versatile enzyme that nonspecifically cleaves double-stranded DNA (dsDNA) to release 5′-phosphorylated di-, tri-, and oligonucleotide products. DNase I has optimal activity in buffers containing Mn2+, Mg2+ and Ca2+, but no other salts. The purpose of the DNase I digestion step is to fragment a large DNA genome into smaller species comprising a library. The cleavage characteristics of DNase I will result in random digestion of template DNA (i.e., no sequence bias) and in the predominance of blunt-ended dsDNA fragments when used in the presence of manganese-based buffers (Melgar, E. and D. A. Goldthwait. 1968. Deoxyribonucleic acid nucleases. II. The effects of metal on the mechanism of action of deoxyribonuclease I. J. Biol. Chem. 243: 4409). The range of digestion products generated following DNase I treatment of genomic templates is dependent on three factors: i) amount of enzyme used (units); ii) temperature of digestion (0° C.); and iii) incubation time (minutes). The DNase I digestion conditions outlined below have been optimized to yield genomic libraries with a size range from 50-700 base pairs (bp).

In a preferred embodiment, the DNase I digests large template DNA or whole genome DNA for 1-2 minutes to generate a population of polynucleotides. In another preferred embodiment, the DNase I digestion is performed at a temperature between 10° C-37° C. In yet another preferred embodiment, the digested DNA fragments are between 50 bp to 700 bp in length.

Polishing

Digestion of genomic DNA (gDNA) templates with DNase I in the presence of Mn2+ will yield fragments of DNA that are either blunt-ended or have protruding termini with one or two nucleotides in length. In a preferred embodiment, an increased number of blunt ends are created with Pfu DNA polymerase. In other embodiments, blunt ends can be created with less efficient DNA polymerases such as T4 DNA polymerase or Klenow DNA polymerase. Pfu “polishing” or blunt ending is used to increase the amount of blunt-ended species generated following genomic template digestion with DNase I. Use of Pfu DNA polymerase for fragment polishing will result in the fill-in of 5′ overhangs. Additionally, Pfu DNA polymerase does not exhibit DNA extendase activity but does have 3′→5′ exonuclease activity that will result in the removal of single and double nucleotide extensions to further increase the amount of blunt-ended DNA fragments available for adaptor ligation (Costa, G. L. and M. P. Weiner. 1994a. Protocols for cloning and analysis of blunt-ended PCR-generated DNA fragments. PCR Methods Appl 3(5):S95; Costa, G. L., A. Grafsky and M. P. Weiner. 1994b. Cloning and analysis of PCR-generated DNA fragments. PCR Methods Appl 3(6):338; Costa, G. L. and M. P. Weiner. 1994c. Polishing with T4 or Pfu polymerase increases the efficiency of cloning of PCR products. Nucleic Acids Res. 22(12):2423).

Adaptor Ligation

If the libraries of nucleic acids are to be attached to the solid substrate, then preferably the nucleic acid templates are annealed to anchor primer sequences using recognized techniques (see, e.g., Hatch, et al., 1999. Genet. Anal. Biomol. Engineer. 15: 35-40; Kool, U.S. Pat. No. 5,714, 320 and Lizardi, U.S. Pat. No. 5,854,033). In general, any procedure for annealing the anchor primers to the template nucleic acid sequences is suitable as long as it results in formation of specific, i.e., perfect or nearly perfect, complementarity between the adapter region or regions in the anchor primer sequence and a sequence present in the template library.

In a preferred embodiment, following fragmentation and blunt ending of the DNA library, universal adaptor sequences are added to each DNA fragment. The universal adaptors are designed to include a set of unique PCR priming regions that are typically 20 bp in length located adjacent to a set of unique sequencing priming regions that are typically 20 bp in length optionally followed by a unique discriminating key sequence consisting of at least one of each of the four deoxyribonucleotides (i.e., A, C, G, T). In a preferred embodiment, the discriminating key sequence is 4 bases in length. In another embodiment, the discriminating key sequence may be combinations of 1-4 bases. In yet another embodiment, each unique universal adaptor is forty-four bp (44 bp) in length. In a preferred embodiment the universal adaptors are ligated, using T4 DNA ligase, onto each end of the DNA fragment to generate a total nucleotide addition of 88 bp to each DNA fragment. Different universal adaptors are designed specifically for each DNA library preparation and will therefore provide a unique identifier for each organism. The size and sequence of the universal adaptors may be modified as would be apparent to one of skill in the art.

For example, to prepare two distinct universal adaptors (i.e., “first” and “second”), single-stranded oligonucleotides may be ordered from a commercial vendor (i.e., Integrated DNA Technologies, IA or Operon Technologies, CA). In one embodiment, the universal adaptor oligonucleotide sequences are modified during synthesis with two or three phosphorothioate linkages in place of phosphodiester linkages at both the 5′ and 3′ ends. Unmodified oligonucleotides are subject to rapid degradation by nucleases and are therefore of limited utility. Nucleases are enzymes that catalyze the hydrolytic cleavage of a polynucleotide chain by cleaving the phosphodiester linkage between nucleotide bases. Thus, one simple and widely used nuclease-resistant chemistry available for use in oligonucleotide applications is the phosphorothioate modification. In phosphorothioates, a sulfur atom replaces a non-bridging oxygen in the oligonucleotide backbone making it resistant to all forms of nuclease digestion (i.e. resistant to both endonuclease and exonuclease digestion). Each oligonucleotide is HPLC-purified to ensure there are no contaminating or spurious oligonucleotide sequences in the synthetic oligonucleotide preparation. The universal adaptors are designed to allow directional ligation to the blunt-ended, fragmented DNA. Each set of double-stranded universal adaptors are designed with a PCR priming region that contains noncomplementary 5′ four-base overhangs that cannot ligate to the blunt-ended DNA fragment as well as prevent ligation with each other at these ends. Accordingly, binding can only occur between the 3′ end of the adaptor and the 5′ end of the DNA fragment or between the 3′ end of the DNA fragment and the 5′ end of the adaptor. Double-stranded universal adaptor sequences are generated by using single-stranded oligonucleotides that are designed with sequences that allow primarily complimentary oligonucleotides to anneal, and to prevent cross-hybridization between two non-complimentary oligonucleotides. In one embodiment, 95% of the universal adaptors are formed from the annealing of complimentary oligonucleotides. In a preferred embodiment, 97% of the universal adaptors are formed from the annealing of complimentary oligonucleotides. In a more preferred embodiment, 99% of the universal adaptors are formed from the annealing of complimentary. oligonucleotides. In a most preferred embodiment, 100% of the universal adaptors are formed from the annealing of complimentary oligonucleotides.

One of the two adaptors can be linked to a support binding moiety. In a preferred embodiment, a 5′ biotin is added to the first universal adaptor to allow subsequent isolation of ssDNA template and noncovalent coupling of the universal adaptor to the surface of a solid support that is saturated with a biotin-binding protein (i.e. streptavidin, neutravidin or avidin). Other linkages are well known in the art and may be used in place of biotin-streptavidin (for example antibody/antigen-epitope, receptor/ligand and oligonucleotide pairing or complimentarily) one embodiment, the solid support is a bead, preferably a polystyrene bead. In one preferred embodiment, the bead has a diameter of about 2.8 μm. As used herein, this bead is referred to as a “sample prep bead”.

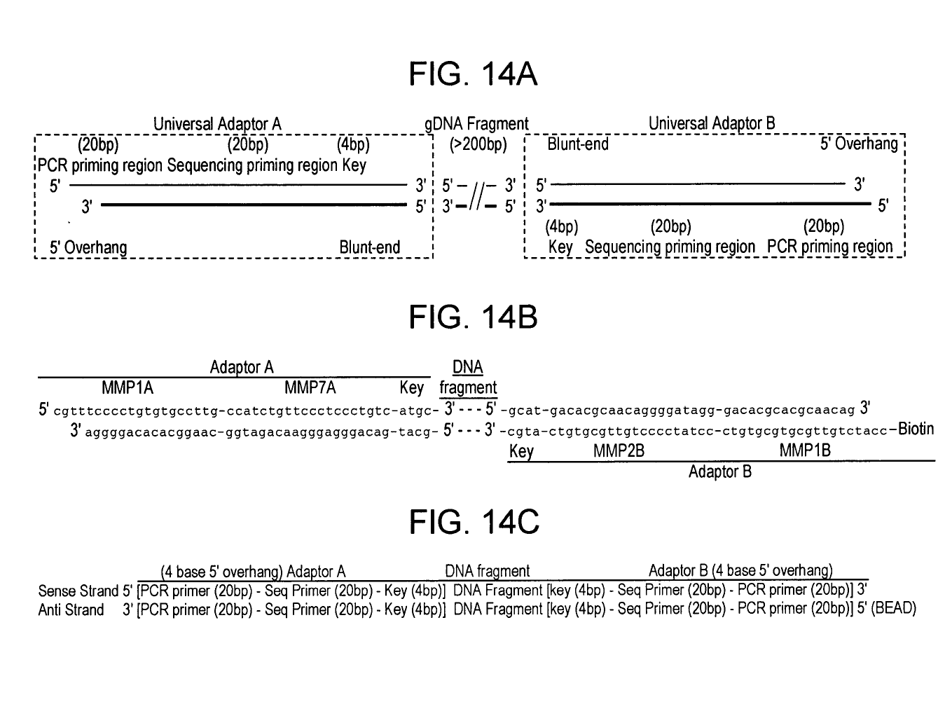

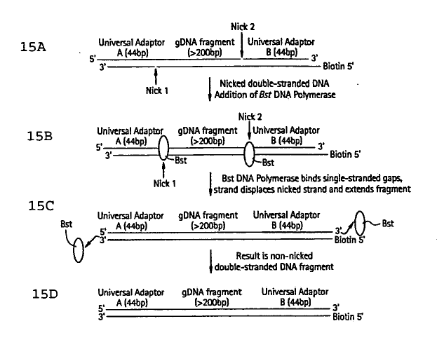

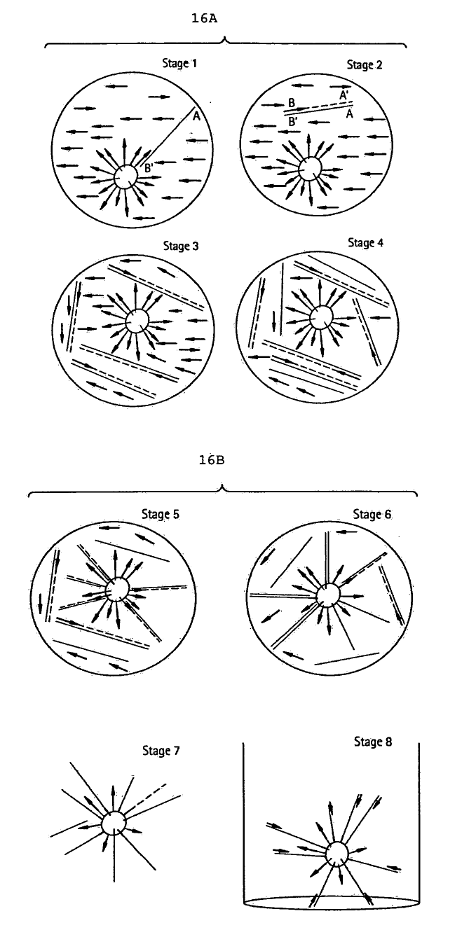

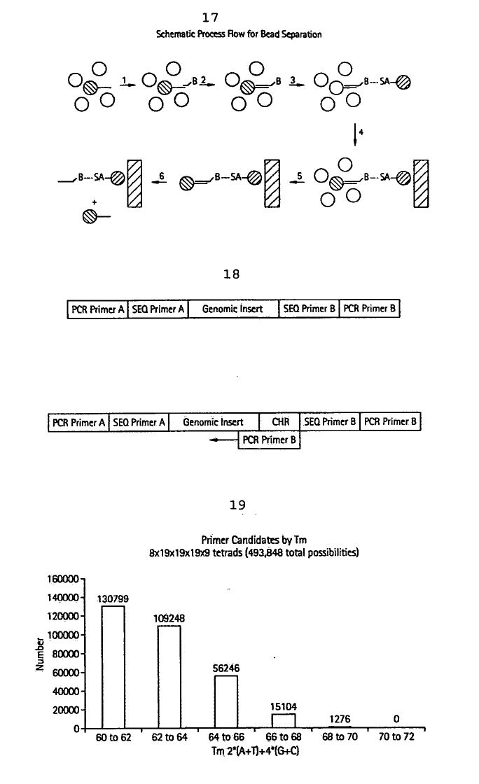

Each universal adaptor may be prepared by combining and annealing two ssDNA oligonucleotides, one containing the sense sequence and the second containing the antisense (complementary) sequence. Schematic representation of the universal adaptor design is outlined in FIG. 14.

Isolation of Ligation Products

The universal adaptor ligation results in the formation of fragmented DNAs with adaptors on each end, unbound single adaptors, and adaptor dimers. In a preferred embodiment, agarose gel electrophoresis is used as a method to separate and isolate the adapted DNA library population from the unligated single adaptors and adaptor dimer populations. In other embodiments, the fragments may be separated by size exclusion chromatography or sucrose sedimentation. The procedure of DNase I digestion of DNA typically yields a library population that ranges from 50-700 bp. In a preferred embodiment, upon conducting agarose gel electrophoresis in the presence of a DNA marker, the addition of the 88 bp universal adaptor set will shift the DNA library population to a larger size and will result in a migration profile in the size range of approximately 130-800 bp; adaptor dimers will migrate at 88 bp; and adaptors not ligated will migrate at 44 bp. Therefore, numerous double-stranded DNA libraries in sizes ranging from 200-800 bp can be physically isolated from the agarose gel and purified using standard gel extraction techniques. In one embodiment, gel isolation of the adapted ligated DNA library will result in the recovery of a library population ranging in size from 200-400 bp. A size of 200-400 bp is ideal for complete DNA sequencing of a genome. However, any size greater than 20 bp will work for Sequence-Based Karyotyping. Other methods of distinguishing adaptor-ligated fragments are known to one of skill in the art.

Nick Repair

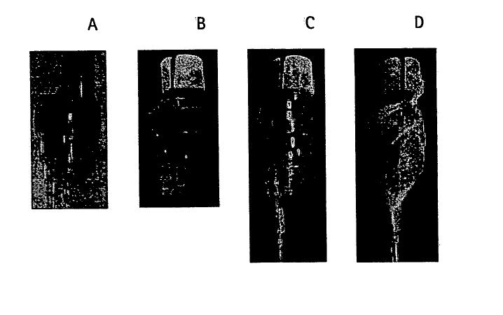

Because the DNA oligonucleotides used for the universal adaptors are not 5′ phosphorylated, gaps will be present at the 3′ junctions of the fragmented DNAs following ligase treatment (see FIG. 15). These two “gaps” or “nicks” can be filled in by using a DNA polymerase enzyme that can bind to, strand displace and extend the nicked DNA fragments. DNA polymerases that lack 3′→5′ exonuclease activity but exhibit 5′→3′ exonuclease activity have the ability to recognize nicks, displace the nicked strands, and extend the strand in a manner that results in the repair of the nicks and in the formation of non-nicked double-stranded DNA (see FIG. 15) (Hamilton, S. C., J. W. Farchaus and M. C. Davis. 2001. DNA polymerases as engines for biotechnology. BioTechniques 31:370).

Several modifying enzymes are utilized for the nick repair step, including but not limited to polymerase, ligase and kinase. DNA polymerases that can be used for this application include, for example, E. coli DNA pol I, Thermoanaerobacter thermohydrosulfuricus pol I, and bacteriophage phi 29. In a preferred embodiment, the strand displacing enzyme Bacillus stearothermophilus pol I (Bst DNA polymerase I) is used to repair the nicked dsDNA and results in non-nicked dsDNA (see FIG. 15). In another preferred embodiment, the ligase is T4 and the kinase is polynucleotide kinase.

Isolation of Single-Stranded DNA

Following the generation of non-nicked dsDNA, ssDNAs comprising both the first and second adaptor molecules are to be isolated (desired populations are designated below with asterisks; “A” and “B” correspond to the first and second adaptors). Double-stranded DNA libraries will have adaptors bound in the following configurations:
Universal Adaptor A—DNA fragment—Universal Adaptor A
Universal Adaptor B—DNA fragment—Universal Adaptor A*
Universal Adaptor A—DNA fragment—Universal Adaptor B*
Universal Adaptor B—DNA fragment—Universal Adaptor B

Universal adaptors are designed such that only one universal adaptor has a 5′ biotin moiety. For example, if universal adaptor B has a 5′ biotin moiety, streptavidin-coated sample prep beads can be used to bind all double-stranded DNA library species with universal adaptor B. Genomic library populations that contain two universal adaptor A species will not contain a 5′ biotin moiety and will not bind to streptavidin-containing sample prep beads and thus can be washed away. The only species that will remain attached to beads are those with universal adaptors A and B and those with two universal adaptor B sequences. DNA species with two universal adaptor B sequences (i.e., biotin moieties at each 5′ end) will be bound to streptavidin-coated sample prep beads at each end, as each strand comprised in the double strand will be bound. Double-stranded DNA species with a universal adaptor A and a universal adaptor B will contain a single 5′biotin moiety and thus will be bound to streptavidin-coated beads at only one end. The sample prep beads are magnetic, therefore, the sample prep beads will remain coupled to a solid support when magnetized. Accordingly, in the presence of a low-salt (“melt” or denaturing) solution, only those DNA fragments that contain a single universal adaptor A and a single universal adaptor B sequence will release the complementary unbound strand. This single-stranded DNA population may be collected and quantitated by, for example, pyrophosphate sequencing, real-time quantitative PCR, agarose gel electrophoresis or capillary gel electrophoresis.

Attachment of Template to Beads

In one embodiment, ssDNA libraries that are created according to the methods of the invention are quantitated to calculate the number of molecules per unit volume. These molecules are annealed to a solid support (bead) that contain oligonucleotide capture primers that are complementary to the PCR priming regions of the universal adaptor ends of the ssDNA species. Beads are then transferred to an amplification protocol. Clonal populations of single species captured on DNA beads may then sequenced. In one embodiment, the solid support is a bead, preferably a sepharose bead. As used herein, this bead is referred to as a “DNA capture bead”.

The beads used herein may be of any convenient size and fabricated from any number of known materials. Example of such materials include: inorganics, natural polymers, and synthetic polymers. Specific examples of these materials include: cellulose, cellulose derivatives, acrylic resins, glass; silica gels, polystyrene, gelatin, polyvinyl pyrrolidone, co-polymers of vinyl and acrylamide, polystyrene cross-linked with divinylbenzene or the like (see, Merrifield Biochemistry 1964, 3, 1385-1390), polyacrylamides, latex gels, polystyrene, dextran, rubber, silicon, plastics, nitrocellulose, celluloses, natural sponges, silica gels, glass, metals plastic, cellulose, cross-linked dextrans (e.g., Sephadex™) and agarose gel (Sepharose™) and solid phase supports known to those of skill in the art. In one embodiment, the diameter of the DNA capture bead is in the range of 20-70 μm. In a preferred embodiment, the diameter of the DNA capture bead is in a range of 20-50 μm. In a more preferred embodiment, the diameter of the DNA capture bead is about 30 μm.

In one aspect, the invention includes a method for generating a library of solid supports comprising: (a) preparing a population of ssDNA templates according to the methods disclosed herein; (b) attaching each DNA template to a solid support such that there is one molecule of DNA per solid support; (c) amplifying the population of single-stranded templates such that the amplification generates a clonal population of each DNA fragment on each solid support; (d) sequencing clonal populations of beads.

In one embodiment, the solid support is a DNA capture bead. In another embodiment, the DNA is genomic DNA, cDNA or reverse transcripts of viral RNA. The DNA may be attached to the solid support, for example, via a biotin-streptavidin linkage, a covalent linkage or by complementary oligonucleotide hybridization. In one embodiment, each DNA template is ligated to a set of universal adaptors. In another embodiment, the universal adaptor pair comprises a common PCR primer sequence, a common sequencing primer sequence and a discriminating key sequence. Single-stranded DNAs are isolated that afford unique ends; single stranded molecules are then attached to a solid support and exposed to amplification techniques for clonal expansion of populations. The DNA may be amplified by PCR.

In another aspect, the invention provides a library of solid supports made by the methods described herein.

The nucleic acid template (e.g., DNA template) prepared by this method may be used for many molecular biological procedures, such as linear extension, rolling circle amplification, PCR and sequencing. This method can be accomplished in a linkage reaction, for example, by using a high molar ratio of bead to DNA. Capture of single-stranded DNA molecules will follow a poisson distribution and will result in a subset of beads with no DNA attached and a subset of beads with two molecules of DNA attached. In a preferred embodiment, there would be one bead to one molecule of DNA. In addition, it is possible to include additional components in the adaptors that may be useful for additional manipulations of the isolated library.

### 2. Nucleic Acid Template Amplification

In order for the nucleic acid template to be sequenced according to one of the methods of this invention the copy number must be amplified to generate a sufficient number of copies of the template to produce a detectable signal by the light detection means. Any suitable nucleic acid amplification means may be used.

A number of in vitro nucleic acid amplification techniques have been described. These amplification methodologies may be differentiated into those methods: (i) which require temperature cycling—polymerase chain reaction (PCR) (see e.g., Saiki, et al., 1995. Science 230: 1350-1354), ligase chain reaction (see e.g., Barany, 1991. Proc. Natl. Acad. Sci. USA 88: 189-193; Barringer, et al., 1990. Gene 89: 117-122) and transcription-based amplification (see e.g., Kwoh, et al., 1989. Proc. Natl. Acad. Sci. USA 86: 1173-1177) and (ii) isothermal amplification systems—self-sustaining, sequence replication (see e.g., Guatelli, et al., 1990. Proc. Natl. Acad. Sci. USA 87: 1874-1878); the Qβ replicase system (see e.g., Lizardi, et al., 1988. BioTechnology 6: 1197-1202); strand displacement amplification Nucleic Acids Res. Apr. 11, 1992;20(7):1691-6.; and the methods described in PNAS Jan. 1, 1992;89(1):392-6; and NASBA J Virol Methods. 1991 Dec;35(3):273-86.

In one embodiment, isothermal amplification is used. Isothermal amplification also includes rolling circle-based amplification (RCA). RCA is discussed in, e.g., Kool, U.S. Pat. No. 5,714,320 and Lizardi, U.S. Pat. No. 5,854,033; Hatch, et al., 1999. Genet. Anal. Biomol. Engineer. 15: 35-40. The result of the RCA is a single DNA strand extended from the 3′ terminus of the anchor primer (and thus is linked to the solid support matrix) and including a concatamer containing multiple copies of the circular template annealed to a primer sequence. Typically, 1,000 to 10,000 or more copies of circular templates, each having a size of, e.g., approximately 30-500, 50-200, or 60-100 nucleotides size range, can be obtained with RCA.

Bead Emulsion PCR Amplification

In a preferred embodiment, a PCR amplification step is performed prior to distribution of the nucleic acid templates onto the picotiter plate.

In a particularly preferred embodiment, a novel amplification system, herein termed “bead emulsion amplification” is performed by attaching a template nucleic acid (e.g., DNA) to be amplified to a solid support, preferably in the form of a generally spherical bead. A library of single stranded template DNA prepared according to the sample preparation methods of this invention is an example of one suitable source of the starting nucleic acid template library to be attached to a bead for use in this amplification method.

The bead is linked to a large number of a single primer species (i.e., primer B in FIG. 16) that is complementary to a region of the template DNA. Template DNA annealed to the bead bound primer. The beads are suspended in aqueous reaction mixture and then encapsulated in a water-in-oil emulsion. The emulsion is composed of discrete aqueous phase microdroplets, approximately 60 to 200 um in diameter, enclosed by a thermostable oil phase. Each microdroplet contains, preferably, amplification reaction solution (i.e., the reagents necessary for nucleic acid amplification). An example of an amplification would be a PCR reaction mix (polymerase, salts, dNTPs) and a pair of PCR primers (primer A and primer B). See, FIG. 16. A subset of the microdroplet population also contains the DNA bead comprising the DNA template. This subset of microdroplet is the basis for the amplification. The microcapsules that are not within this subset have no template DNA and will not participate in amplification. In one embodiment, the amplification technique is PCR and the PCR primers are present in a 8:1 or 16:1 ratio (i.e., 8 or 16 of one primer to 1 of the second primer) to perform asymmetric PCR.

In this overview, the DNA is annealed to an oligonucleotide (primer B) which is immobilized to a bead. During thermocycling (FIG. 16), the bond between the single stranded DNA template and the immobilized B primer on the bead is broken, releasing the template into the surrounding microencapsulated solution. The amplification solution, in this case, the PCR solution, contains addition solution phase primer A and primer B. Solution phase B primers readily bind to the complementary b′ region of the template as binding kinetics are more rapid for solution phase primers than for immobilized primers. In early phase PCR, both A and B strands amplify equally well (FIG. 16).

By midphase PCR (i.e., between cycles 10 and 30) the B primers are depleted, halting exponential amplification. The reaction then enters asymmetric amplification and the amplicon population becomes dominated by A strands (FIG. 16). In late phase PCR (FIG. 16), after 30 to 40 cycles, asymmetric amplification increases the concentration of A strands in solution. Excess A strands begin to anneal to bead immobilized B primers. Thermostable polymerases then utilize the A strand as a template to synthesize an immobilized, bead bound B strand of the amplicon.

In final phase PCR (FIG. 16), continued thermal cycling forces additional annealing to bead bound primers. Solution phase amplification may be minimal at this stage but concentration of immobilized B strands increase. Then, the emulsion is broken and the immobilized product is rendered single stranded by denaturing (by heat, pH etc.) which removes the complimentary A strand. The A primers are annealed to the A′ region of immobilized strand, and immobilized strand is loaded with sequencing enzymes, and any necessary accessory proteins. The beads are then sequenced using recognized pyrophosphate techniques (described, e.g., in U.S. Pat. No. 6,274,320, 6258,568 and 6,210,891, incorporated in toto herein by reference).

Template Design

In a preferred embodiment, the DNA template to be amplified by bead emulsion amplification can be a population of DNA such as, for example, a genomic DNA library or a cDNA library. It is preferred that each member of the population have a common nucleic acid sequence at the first end and a common nucleic acid sequence at a second end. This can be accomplished, for example, by ligating a first adaptor DNA sequence to one end and a second adaptor DNA sequence to a second end of the DNA population. Many DNA and cDNA libraries, by nature of the cloning vector (e.g., Bluescript, Stratagene, La Jolla, Calif.) fit this description of having a common sequence at a first end and a second common sequence at a second end of each member DNA. The DNA template may be of any size amenable to in vitro amplification (including the preferred amplification techniques of PCR and asymmetric PCR). In a preferred embodiment, the DNA template is between about 150 to 750 bp in size, such as, for example about 250 bp in size.

Binding Nucleic Acid Template to Capture Beads

In a first step, a single stranded nucleic acid template to be amplified is attached to a capture bead. The nucleic acid template may be attached to the solid support capture bead in any manner known in the art. Numerous methods exist in the art for attaching DNA to a solid support such as the preferred microscopic bead. According to the present invention, covalent chemical attachment of the DNA to the bead can be accomplished by using standard coupling agents, such as water-soluble carbodiimide, to link the 5′-phosphate on the DNA to amine-coated capture beads through a phosphoamidate bond. Another alternative is to first couple specific oligonucleotide linkers to the bead using similar chemistry, and to then use DNA ligase to link the DNA to the linker on the bead. Other linkage chemistries to join the oligonucleotide to the beads include the use of N-hydroxysuccinamide (NHS) and its derivatives. In such a method, one end of the oligonucleotide may contain a reactive group (such as an amide group) which forms a covalent bond with the solid support, while the other end of the linker contains a second reactive group that can bond with the oligonucleotide to be immobilized. In a preferred embodiment, the oligonucleotide is bound to the DNA capture bead by covalent linkage. However, non-covalent linkages, such as chelation or antigen-antibody complexes, may also be used to join the oligonucleotide to the bead.

Oligonucleotide linkers can be employed which specifically hybridize to unique sequences at the end of the DNA fragment, such as the overlapping end from a restriction enzyme site or the “sticky ends” of bacteriophage lambda based cloning vectors, but blunt-end ligations can also be used beneficially. These methods are described in detail in U.S. Pat. No. 5,674,743. It is preferred that any method used to immobilize the beads will continue to bind the immobilized oligonucleotide throughout the steps in the methods of the invention.

In one embodiment, each capture bead is designed to have a plurality of nucleic acid primers that recognize (i.e., are complementary to) a portion of the nucleic template, and the nucleic acid template is thus hybridized to the capture bead. In the methods described herein, clonal amplification of the template species is desired, so it is preferred that only one unique nucleic acid template is attached to any one capture bead.

The beads used herein may be of any convenient size and fabricated from any number of known materials. Example of such materials include: inorganics, natural polymers, and synthetic polymers. Specific examples of these materials include: cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (as described, e.g., in Merrifield, Biochemistry 1964, 3, 1385-1390), polyacrylamides, latex gels, polystyrene, dextran, rubber, silicon, plastics, nitrocellulose, natural sponges, silica gels, control pore glass, metals, cross-linked dextrans (e.g., Sephadex™) agarose gel (Sepharose™), and solid phase supports known to those of skill in the art. In a preferred embodiment, the capture beads are Sepharose beads approximately 25 to 40 μm in diameter.

Emulsification

Capture beads with attached single strand template nucleic acid are emulsified as a heat stable water-in-oil emulsion. The emulsion may be formed according to any suitable method known in the art. One method of creating emulsion is described below but any method for making an emulsion may be used. These methods are known in the art and include adjuvant methods, counterflow methods, crosscurrent methods, rotating drum methods, and membrane methods. Furthermore, the size of the microcapsules may be adjusted by varying the flow rate and speed of the components. For example, in dropwise addition, the size of the drops and the total time of delivery may be varied. Preferably, the emulsion contains a density of bead “microreactors” at a density of about 3,000 beads per microliter.

The emulsion is preferably generated by suspending the template-attached beads in amplification solution. As used herein, the term “amplification solution” means the sufficient mixture of reagents that is necessary to perform amplification of template DNA. One example of an amplification solution, a PCR amplification solution, is provided in the Examples below—it will be appreciated that various modifications may be made to the PCR solution.

In one embodiment, the bead/amplification solution mixture is added dropwise into a spinning mixture of biocompatible oil (e.g., light mineral oil, Sigma) and allowed to emulsify. The oil used may be supplemented with one or more biocompatible emulsion stabilizers. These emulsion stabilizers may include Atlox 4912, Span 80, and other recognized and commercially available suitable stabilizers. Preferably, the droplets formed range in size from 5 micron to 500 microns, more preferably, from between about 50 to 300 microns, and most preferably, from 100 to 150 microns.

There is no limitation in the size of the microreactors. The microreactors should be sufficiently large to encompass sufficient amplification reagents for the degree of amplification required. However, the microreactors should be sufficiently small so that a population of microreactors, each containing a member of a DNA library, can be amplified by conventional laboratory equipment (e.g., PCR thermocycling equipment, test tubes, incubators and the like).

With the limitations described above, the optimal size of a microreactor may be between 100 to 200 microns in diameter. Microreactors of this size would allow amplification of a DNA library comprising about 600,000 members in a suspension of microreactors of less than 10 ml in volume. For example, if PCR was the chosen amplification method, 10 mls would fit in 96 tubes of a regular thermocycler with 96 tube capacity. In a preferred embodiment, the suspension of 600,000 microreactors would have a volume of less than 1 ml. A suspension of less than 1 ml may be amplified in about 10 tubes of a conventional PCR thermocycler. In a most preferred embodiment, the suspension of 600,000 microreactors would have a volume of less than 0.5 ml.

Amplification

After encapsulation, the template nucleic acid may be amplified by any suitable method of DNA amplification including transcription-based amplification systems (Kwoh D. et al., Proc. Natl. Acad Sci. (U.S.A.) 86:1173 (1989); Gingeras T. R. et al., PCT appl. WO 88/10315; Davey, C. et al., European Patent Application Publication No. 329,822; Miller, H. I. et al., PCT appl. WO 89/06700, and “race” (Frohman, M. A., In: PCR Protocols: A Guide to Methods and Applications, Academic Press, NY (1990)) and “one-sided PCR” (Ohara, O. et al., Proc. Natl. Acad. Sci. (U.S.A.) 86.5673-5677 (1989)). Still other less common methods such as “di-oligonucleotide” amplification, isothermal amplification (Walker, G. T. et al., Proc. Natl. Acad. Sci. (U.S.A.) 89:392-396 (1992)), and rolling circle amplification (reviewed in U.S. Pat. No. 5,714,320), may be used in the present invention.

In a preferred embodiment, DNA amplification is performed by PCR. PCR according to the present invention may be performed by encapsulating the target nucleic acid, bound to a bead, with a PCR solution comprising all the necessary reagents for PCR. Then, PCR may be accomplished by exposing the emulsion to any suitable thermocycling regimen known in the art. In a preferred embodiment, between 30 and 50 cycles, preferably about 40 cycles, of amplification are performed. It is desirable, but not necessary, that following the amplification procedure there be one or more hybridization and extension cycles following the cycles of amplification. In a preferred embodiment, between 10 and 30 cycles, preferably about 25 cycles, of hybridization and extension are performed (e.g., as described in the examples). Routinely, the template DNA is amplified until typically at least two million to fifty million copies, preferably about ten million to thirty million copies of the template DNA are immobilized per bead.

Breaking the Emulsion and Bead Recovery

Following amplification of the template, the emulsion is “broken” (also referred to as “demulsification” in the art). There are many methods of breaking an emulsion (see, e.g., U.S. Pat. No. 5,989,892 and references cited therein) and one of skill in the art would be able to select the proper method. In the present invention, one preferred method of breaking the emulsion is to add additional oil to cause the emulsion to separate into two phases. The oil phase is then removed, and a suitable organic solvent (e.g., hexanes) is added. After mixing, the oil/organic solvent phase is removed. This step may be repeated several times. Finally, the aqueous layers above the beads are removed. The beads are then washed with an organic solvent/annealing buffer mixture (e.g., one suitable annealing buffer is described in the examples), and then washed again in annealing buffer. Suitable organic solvents include alcohols such as methanol, ethanol and the like.

The amplified template-containing beads may then be resuspended in aqueous solution for use, for example, in a sequencing reaction according to known technologies. (See, Sanger, F. et al., Proc. Natl. Acad. Sci. U.S.A. 75, 5463-5467 (1977); Maxam, A. M. & Gilbert, W. Proc Natl Acad Sci USA 74, 560-564 (1977); Ronaghi, M. et al., Science 281, 363, 365 (1998); Lysov, I. et al., Dokl Akad Nauk SSSR 303, 1508-1511 (1988); Bains W. & Smith G. C. J.TheorBiol 135, 303-307(1988); Drnanac, R. et al., Genomics 4, 114-128 (1989); Khrapko, K. R. et al., FEBS Lett 256. 118-122 (1989); Pevzner P. A. J Biomol Struct Dyn 7, 63-73 (1989); Southern, E. M. et al., Genomics 13, 1008-1017 (1992).) If the beads are to be used in a pyrophosphate-based sequencing reaction (described, e.g., in U.S. Pat. Nos. 6,274,320, 6258,568 and 6,210,891, and incorporated in toto herein by reference), then it is necessary to remove the second strand of the PCR product and anneal a sequencing primer to the single stranded template that is bound to the bead.

Briefly, the second strand is melted away using any number of commonly known methods such as NaOH, low ionic (e.g., salt) strength, or heat processing. Following this melting step, the beads are pelleted and the supernatant is discarded. The beads are resuspended in an annealing buffer, the sequencing primer added, and annealed to the bead-attached single stranded template using a standard annealing cycle.

Purifying the Beads

At this point, the amplified DNA on the bead may be sequenced either directly on the bead or in a different reaction vessel. In an embodiment of the present invention, the DNA is sequenced directly on the bead by transferring the bead to a reaction vessel and subjecting the DNA to a sequencing reaction (e.g., pyrophosphate or Sanger sequencing). Alternatively, the beads may be isolated and the DNA may be removed from each bead and sequenced. In either case, the sequencing steps may be performed on each individual bead. However, this method, while commercially viable and technically feasible, may not be most effective because many of the beads will be negative beads (a bead that does not have amplified DNA attached). Accordingly, the following optional process may be used for removing beads that contain no nucleic acid template prior to distribution onto the picotiter plate.

A high percentage of the beads may be “negative” (i.e., have no amplified nucleic acid template attached thereto) if the goal of the initial DNA attachment is to minimize beads with two different copies of DNA. For useful pyrophosphate sequencing, each bead should contain multiple copies of a single species of DNA. This requirement is most closely approached by maximizing the total number of beads with a single fragment of DNA bound (before amplification). This goal can be achieved by the observation of a mathematical model.

For the general case of “N” number of DNAs randomly distributed among M number of beads, the relative bead population containing any number of DNAs depends on the ratio of N/M. The fraction of beads containing N DNAs R(N) may be calculated using the Poisson distribution:
R(N)=exp-(N/M)×(N/M)^{N}/N! (where × is the multiplication symbol)

The table below shows some calculated values for various N/M (the average DNA fragment to bead ratio) and N (the number of fragments actually bound to a bead).

N/M

| | 0.1 | 0.5 | 1 | 2 |
|---|---|---|---|---|
| R(0) | 0.9 | 0.61 | 0.37 | 0.13 |
| R(1) | 0.09 | 0.3 | 0.37 | 0.27 |
| R(N > 1) | 0.005 | 0.09 | 0.26 | 0.59 |

In the table the top row denotes the various ratios of N/M. R(0) denotes the fraction of beads with no DNA, R(1) denotes the fraction of beads with one DNA attached (before amplification) and R(N>1) denotes the fraction of DNA with more than one DNA attached (before amplification).

The table indicates that the maximum fraction of beads containing a single DNA fragment is 0.37 (37%) and occurs at a fragment to bead ratio of one. In this mixture, about 63% of the beads is useless for sequencing because they have either no DNA or more than a single species of DNA. Additionally, controlling the fragment to bead ratio require complex calculations and variability could produce bead batches with a significantly smaller fraction of useable beads.

This inefficiency could be significantly ameliorated if beads containing amplicon (originating from the binding of at least one fragment) could be separated from those without amplicon (originating from beads with no bound fragments). An amplicon is defined as any nucleic acid molecules produced by an in vitro nucleic amplification technique. Binding would be done at low average fragment-to-bead ratios (N/M<1), minimizing the ratio of beads with more than one DNA bound. A separation step would remove most or all of the beads with no DNA leaving an enriched population of beads with one species of amplified DNA. These beads may be applied to any method of sequencing such as, for example, pyrophosphate sequencing. Because the fraction of beads with one amplicon (N=1) has been enriched, any method of sequencing would be more efficient.

As an example, with an average fragment to bead ratio of 0.1, 90% of the beads will have no amplicon, 9% of the beads would be useful with one amplicon, and 0.5% of the beads will have more than one amplicon. An enrichment process of the invention will remove the 90% of the zero amplicon beads leaving a population of beads where the sequenceable fraction (N=1) is:
1-(0.005/0.09)=94%.

Dilution of the fragment to bead mixture, along with separation of beads containing amplicon can yield an enrichment of 2.5 folds over the optimal unenriched method. 94%/37% (see table above N/M=1)=2.5. An additional benefit of the enrichment procedure of the invention is that the ultimate fraction of sequenceable beads is relatively insensitive to variability in N/M. Thus, complex calculations to derive the optimal N/M ratio are either unnecessary or may be performed to a lower level of precision. This will ultimately make the procedure more suitable to performance by less trained personnel or automation. An additional benefit of the procedure is that the zero amplicon beads may be recycled and reused. While recycling is not necessary, it may reduce cost or the total bulk of reagents making the method of the invention more suitable for some purposes such as, for example, portable sampling, remote robotic sampling and the like. In addition, all the benefits of the procedure (i.e., less trained personnel, automation, recycling of reagents) will reduce the cost of the procedure. The procedure is described in more detail below.

The enrichment procedure may be used to treat beads that have been amplified in the bead emulsion method above. The amplification is designed so that each amplified molecule contains the same DNA sequence at its 3′ end. The nucleotide sequence may be a 20 mer but may be any sequence from 15 bases or more such as 25 bases, 30 bases, 35 bases, or 40 bases or longer. Naturally, while longer oligonucleotide ends are functional, they are not necessary. This DNA sequence may be introduced at the end of an amplified DNA by one of skill in the art. For example, if PCR is used for amplification of the DNA, the sequence may be part of one member of the PCR primer pair.

A schematic of the enrichment process is illustrated in FIG. 17. Here, the amplicon-bound bead mixed with 4 empty beads represents the fragment-diluted amplification bead mixture. In step 1, a biotinylated primer complementary to the 3′ end of the amplicon is annealed to the amplicon. In step 2, DNA polymerase and the four natural deoxynucleotides triphosphates (dNTPs) are added to the bead mix and the biotinylated primer is extended. This extension is to enhance the bonding between the biotinylated primer and the bead-bound DNA. This step may be omitted if the biotinylated primer—DNA bond is strong (e.g., in a high ionic environment). In Step 3, streptavidin coated beads susceptible to attraction by a magnetic field (referred to herein as “magnetic streptavidin beads”) are introduced to the bead mixtures. Magnetic beads are commercially available, for example, from Dynal (M290). The streptavidin capture moieties binds biotins hybridized to the amplicons, which then specifically fix the amplicon-bound beads to the magnetic streptavidin beads.

In step 5, a magnetic field (represented by a magnet) is applied near the reaction mixture, which causes all the “magnetic streptavidin beads/amplicon bound bead complexes” to be positioned along one side of the tube most proximal to the magnetic field. Magnetic beads without amplicon bound beads attached are also expected to be positioned along the same side. Beads without amplicons remain in solution. The bead mixture is washed and the beads not immobilized by the magnet (i.e., the empty beads) are removed and discarded. In step 6, the extended biotinylated primer strand is separated from the amplicon strand by “melting”—a step that can be accomplished, for example, by heat or a change in pH. The heat may be 60° C. in low salt conditions (e.g., in a low ionic environment such as 0.1×SSC). The change in pH may be accomplished by the addition of NaOH. The mixture is then washed and the supernatant, containing the amplicon bound beads, is recovered while the now unbound magnetic beads are retained by a magnetic field. The resultant enriched beads may be used for DNA sequencing. It is noted that the primer on the DNA capture bead may be the same as the primer of step 2 above. In this case, annealing of the amplicon-primer complementary strands (with or without extension) is the source of target-capture affinity.

The biotin streptavidin pair could be replaced by a variety of capture-target pairs. Two categories are pairs whose binding can be subsequently cleaved and those which bind irreversibly, under conditions that are practically achievable. Cleavable pairs include thiol-thiol, Digoxigenin/anti-Digoxigenin, -Captavidin™ if cleavage of the target-capture complex is desired.

As described above, step 2 is optional. If step 2 is omitted, it may not be necessary to separate the magnetic beads from the amplicon bound beads. The amplicon bound beads, with the magnetic beads attached, may be used directly for sequencing. If the sequencing were to be performed in a microwell, separation would not be necessary if the amplicon bound bead-magnetic bead complex can fit inside the microwell.

While the use of magnetic capture beads is convenient, capture moieties can be bound to other surfaces. For example, streptavidin could be chemically bound to a surface, such as, the inner surface of a tube. In this case, the amplified bead mixture may be flowed through. The amplicon bound beads will tend to be retained until “melting” while the empty beads will flow through. This arrangement may be particularly advantageous for automating the bead preparation process.

While the embodiments described above is particularly useful, other methods can be envisioned to separate beads. For example, the capture beads may be labeled with a fluorescent moiety which would make the target-capture bead complex fluorescent. The target capture bead complex may be separated by flow cytometry or fluorescence cell sorter. Using large capture beads would allow separation by filtering or other particle size separation techniques. Since both capture and target beads are capable of forming complexes with a number of other beads, it is possible to agglutinate a mass of cross-linked capture-target beads. The large size of the agglutinated mass would make separation possible by simply washing away the unagglutinated empty beads. The methods described are described in more detail, for example, in Bauer, J.; J. Chromatography B, 722 (1999) 55-69 and in Brody et al., Applied Physics Lett. 74 (1999) 144-146.

The DNA capture beads each containing multiple copies of a single species of nucleic acid template prepared according to the above method are then suitable for distribution onto the picotiter plate.

### 3. Sequencing the Nucleic Acid Template

Pyrophosphate sequencing is used according to the methods of this invention to sequence the nucleic acid template. This technique is based on the detection of released pyrophosphate (Ppi) during DNA synthesis. See, e.g., Hyman, 1988. A new method of sequencing DNA. Anal Biochem. 174:423-36; Ronaghi, 2001. Pyrosequencing sheds light on DNA sequencing. Genome Res. 11:3-11.

In a cascade of enzymatic reactions, visible light is generated proportional to the number of incorporated nucleotides. The cascade starts with a nucleic acid polymerization reaction in which inorganic Ppi is released with nucleotide incorporation by polymerase. The released Ppi is converted to ATP by ATP sulfurylase, which provides the energy to luciferase to oxidize luciferin and generates light. Because the added nucleotide is known, the sequence of the template can be determined. Solid-phase pyrophosphate sequencing utilizes immobilized DNA in a three-enzyme system (see Figures). To increase the signal-to-noise ratio, the natural dATP has been replaced by dATPαS. Typically dATPαS is a mixture of two isomers (Sp and Rp); the use of pure 2′-deoxyadenosine-5′-O′-(1-thiotriphosphate) Sp-isomer in pyrophosphate sequencing allows substantially longer reads, up to doubling of the read length.

### 4. Methods of Sequencing Nucleic Acids

Pyrophosphate-based sequencing is then performed. The sample DNA sequence and the extension primer are then subjected to a polymerase reaction in the presence of a nucleotide triphosphate whereby the nucleotide triphosphate will only become incorporated and release pyrophosphate (PPi) if it is complementary to the base in the target position, the nucleotide triphosphate being added either to separate aliquots of sample-primer mixture or successively to the same sample-primer mixture. The release of PPi is then detected to indicate which nucleotide is incorporated.

In one embodiment, a region of the sequence product is determined by annealing a sequencing primer to a region of the template nucleic acid, and then contacting the sequencing primer with a DNA polymerase and a known nucleotide triphosphate, i.e., dATP, dCTP, dGTP, dTTP, or an analog of one of these nucleotides. The sequence can be determined by detecting a sequence reaction byproduct, as is described below.

The sequence primer can be any length or base composition, as long as it is capable of specifically annealing to a region of the amplified nucleic acid template. No particular structure for the sequencing primer is required so long as it is able to specifically prime a region on the amplified template nucleic acid. Preferably, the sequencing primer is complementary to a region of the template that is between the sequence to be characterized and the sequence hybridizable to the anchor primer. The sequencing primer is extended with the DNA polymerase to form a sequence product. The extension is performed in the presence of one or more types of nucleotide triphosphates, and if desired, auxiliary binding proteins.

Incorporation of the dNTP is preferably determined by assaying for the presence of a sequencing byproduct. In a preferred embodiment, the nucleotide sequence of the sequencing product is determined by measuring inorganic pyrophosphate (PPi) liberated from a nucleotide triphosphate (dNTP) as the dNMP is incorporated into an extended sequence primer. This method of sequencing, termed Pyrosequencing™ technology (PyroSequencing AB, Stockholm, Sweden) can be performed in solution (liquid phase) or as a solid phase technique. PPi-based sequencing methods are described generally in, e.g., WO9813523A1, Ronaghi, et al., 1996. Anal. Biochem. 242: 84-89, Ronaghi, et al., 1998. Science 281: 363-365 (1998) and U.S. Ser. No. 2001/0024790. These disclosures of PPi sequencing are incorporated herein in their entirety, by reference. See also, e.g., U.S. Pat. Nos. 6,210,891 and 6,258,568, each fully incorporated herein by reference.

Pyrophosphate released under these conditions can be detected enzymatically (e.g., by the generation of light in the luciferase-luciferin reaction). Such methods enable a nucleotide to be identified in a given target position, and the DNA to be sequenced simply and rapidly while avoiding the need for electrophoresis and the use of potentially dangerous radiolabels.

PPi can be detected by a number of different methodologies, and various enzymatic methods have been previously described (see e.g., Reeves, et al., 1969. Anal. Biochem. 28: 282-287; Guillory, et al., 1971. Anal. Biochem. 39: 170-180; Johnson, et al., 1968. Anal. Biochem. 15: 273; Cook, et al., 1978. Anal. Biochem. 91: 557-565; and Drake, et al., 1979. Anal. Biochem. 94: 117-120).

PPi liberated as a result of incorporation of a dNTP by a polymerase can be converted to ATP using, e.g., an ATP sulfurylase. This enzyme has been identified as being involved in sulfur metabolism. Sulfur, in both reduced and oxidized forms, is an essential mineral nutrient for plant and animal growth (see e.g., Schmidt and Jager, 1992. Ann. Rev. Plant Physiol. Plant Mol. Biol. 43: 325-349). In both plants and microorganisms, active uptake of sulfate is followed by reduction to sulfide. As sulfate has a very low oxidation/reduction potential relative to available cellular reductants, the primary step in assimilation requires its activation via an ATP-dependent reaction (see e.g., Leyh, 1993. Crit. Rev. Biochem. Mol. Biol. 28: 515-542). ATP sulfurylase (ATP: sulfate adenylyltransferase; EC 2.7.7.4) catalyzes the initial reaction in the metabolism of inorganic sulfate (SO₄⁻²); see e.g., Robbins and Lipmann, 1958. J. Biol. Chem. 233: 686-690; Hawes and Nicholas, 1973. Biochem. J. 133: 541-550). In this reaction SO₄⁻² is activated to adenosine 5′-phosphosulfate (APS).

ATP sulfurylase has been highly purified from several sources, such as Saccharomyces cerevisiae (see e.g., Hawes and Nicholas, 1973. Biochem. J. 133: 541-550); Penicillium chrysogenum (see e.g., Renosto, et al., 1990. J. Biol. Chem. 265: 10300-10308); rat liver (see e.g., Yu, et al., 1989. Arch. Biochem. Biophys. 269: 165-174); and plants (see e.g., Shaw and Anderson, 1972. Biochem. J. 127: 237-247; Osslund, et al., 1982. Plant Physiol. 70: 39-45). Furthermore, ATP sulfurylase genes have been cloned from prokaryotes (see e.g., Leyh, et al., 1992. J. Biol. Chem. 267: 10405-10410; Schwedock and Long, 1989. Mol. Plant Microbe Interaction 2: 181-194; Laue and Nelson, 1994. J. Bacteriol. 176: 3723-3729); eukaryotes (see e.g., Cherest, et al., 1987. Mol. Gen. Genet. 210: 307-313; Mountain and Korch, 1991. Yeast 7: 873-880; Foster, et al., 1994. J. Biol. Chem. 269: 19777-19786); plants (see e.g., Leustek, et al., 1994. Plant Physiol. 105: 897-90216); and animals (see e.g., Li, et al., 1995. J. Biol. Chem. 270: 29453-29459). The enzyme is a homo-oligomer or heterodimer, depending upon the specific source (see e.g., Leyh and Suo, 1992. J. Biol. Chem. 267: 542-545).

In some embodiments, a thermostable sulfurylase is used. Thermostable sulfurylases can be obtained from, e.g., Archaeoglobus or Pyrococcus spp. Sequences of thermostable sulfurylases are available at database Acc. No. 028606, Acc. No. Q9YCR4, and Acc. No. P56863.

ATP sulfurylase has been used for many different applications, for example, bioluminometric detection of ADP at high concentrations of ATP (see e.g., Schultz, et al., 1993. Anal. Biochem. 215: 302-304); continuous monitoring of DNA polymerase activity (see e.g., Nyrbn, 1987. Anal. Biochem. 167: 235-238); and DNA sequencing (see e.g., Ronaghi, et al., 1996. Anal. Biochem. 242: 84-89; Ronaghi, et al., 1998. Science 281: 363-365; Ronaghi, et al., 1998. Anal. Biochem. 267: 65-71).

Several assays have been developed for detection of the forward ATP sulfurylase reaction. The colorimetric molybdolysis assay is based on phosphate detection (see e.g., Wilson and Bandurski, 1958. J. Biol. Chem. 233: 975-981), whereas the continuous spectrophotometric molybdolysis assay is based upon the detection of NADH oxidation (see e.g., Seubert, et al., 1983. Arch. Biochem. Biophys. 225: 679-691; Seubert, et al., 1985. Arch. Biochem. Biophys. 240: 509-523). The later assay requires the presence of several detection enzymes. In addition, several radioactive assays have also been described in the literature (see e.g., Daley, et al., 1986. Anal. Biochem. 157: 385-395). For example, one assay is based upon the detection of ³²PPi released from ³²P-labeled ATP (see e.g., Seubert, et al., 1985. Arch. Biochem. Biophys. 240: 509-523) and another on the incorporation of ³⁵S into [³⁵S]-labeled APS (this assay also requires purified APS kinase as a coupling enzyme; see e.g., Seubert, et al., 1983. Arch. Biochem. Biophys. 225: 679-691); and a third reaction depends upon the release of ³⁵SO₄⁻² from [³⁵S]-labeled APS (see e.g., Daley, et al., 1986. Anal. Biochem. 157: 385-395).

For detection of the reversed ATP sulfurylase reaction a continuous spectrophotometric assay (see e.g., Segel, et al., 1987. Methods Enzymol. 143: 334-349); a bioluminometric assay (see e.g., Balharry and Nicholas, 1971. Anal. Biochem. 40: 1-17); an ³⁵SO₄⁻² release assay (see e.g., Seubert, et al., 1985. Arch. Biochem. Biophys. 240: 509-523); and a ³²PPi incorporation assay (see e.g., Osslund, et al., 1982. Plant Physiol. 70: 39-45) have been previously described.

ATP produced by an ATP sulfurylase can be hydrolyzed using enzymatic reactions to generate light. Light-emitting chemical reactions (i.e., chemiluminescence) and biological reactions (i.e., bioluminescence) are widely used in analytical biochemistry for sensitive measurements of various metabolites. In bioluminescent reactions, the chemical reaction that leads to the emission of light is enzyme-catalyzed. For example, the luciferin-luciferase system allows for specific assay of ATP and the bacterial luciferase-oxidoreductase system can be used for monitoring of NAD(P)H. Both systems have been extended to the analysis of numerous substances by means of coupled reactions involving the production or utilization of ATP or NAD(P)H (see e.g., Kricka, 1991. Chemiluminescent and bioluminescent techniques. Clin. Chem. 37: 1472-1281).

The development of new reagents have made it possible to obtain stable light emission proportional to the concentrations of ATP (see e.g., Lundin, 1982. Applications of firefly luciferase In; Luminescent Assays (Raven Press, New York) or NAD(P)H (see e.g., Lovgren, et al., Continuous monitoring of NADH-converting reactions by bacterial luminescence. J. Appl. Biochem. 4: 103-111). With such stable light emission reagents, it is possible to make endpoint assays and to calibrate each individual assay by addition of a known amount of ATP or NAD(P)H. In addition, a stable light-emitting system also allows continuous monitoring of ATP- or NAD(P)H-converting systems.

Suitable enzymes for converting ATP into light include luciferases, e.g., insect luciferases. Luciferases produce light as an end-product of catalysis. The best known light-emitting enzyme is that of the firefly, Photinus pyralis (Coleoptera). The corresponding gene has been cloned and expressed in bacteria (see e.g., de Wet, et al., 1985. Proc. Natl. Acad. Sci. USA 80: 7870-7873) and plants (see e.g., Ow, et al., 1986. Science 234: 856-859), as well as in insect (see e.g., Jha, et al., 1990. FEBS Lett. 274: 24-26) and mammalian cells (see e.g., de Wet, et al., 1987. Mol. Cell. Biol. 7: 725-7373; Keller, et al., 1987. Proc. Natl. Acad. Sci. USA 82: 3264-3268). In addition, a number of luciferase genes from the Jamaican click beetle, Pyroplorus plagiophihalamus (Coleoptera), have recently been cloned and partially characterized (see e.g., Wood, et al., 1989. J. Biolumin. Chemilumin. 4: 289-301; Wood, et al., 1989. Science 244: 700-702). Distinct luciferases can sometimes produce light of different wavelengths, which may enable simultaneous monitoring of light emissions at different wavelengths. Accordingly, these aforementioned characteristics are unique, and add new dimensions with respect to the utilization of current reporter systems.

Firefly luciferase catalyzes bioluminescence in the presence of luciferin, adenosine 5′-triphosphate (ATP), magnesium ions, and oxygen, resulting in a quantum yield of 0.88 (see e.g., McElroy and Selinger, 1960. Arch. Biochem. Biophys. 88: 136-145). The firefly luciferase bioluminescent reaction can be utilized as an assay for the detection of ATP with a detection limit of approximately 1×10⁻¹³ M (see e.g., Leach, 1981. J. Appl. Biochem. 3: 473-517). In addition, the overall degree of sensitivity and convenience of the luciferase-mediated detection systems have created considerable interest in the development of firefly luciferase-based biosensors (see e.g., Green and Kricka, 1984. Talanta 31: 173-176; Blum, et al., 1989. J. Biolumin. Chemilumin. 4: 543-550).

Using the above-described enzymes, the sequence primer is exposed to a polymerase and a known dNTP. If the dNTP is incorporated onto the 3′ end of the primer sequence, the dNTP is cleaved and a PPi molecule is liberated. The PPi is then converted to ATP with ATP sulfurylase. Preferably, the ATP sulfurylase is present at a sufficiently high concentration that the conversion of PPi proceeds with first-order kinetics with respect to PPi. In the presence of luciferase, the ATP is hydrolyzed to generate a photon. The reaction preferably has a sufficient concentration of luciferase present within the reaction mixture such that the reaction, ATP→ADP+PO₄³⁻+photon (light), proceeds with first-order kinetics with respect to ATP. The photon can be measured using methods and apparatuses described below. In one embodiment, the PPi and a coupled sulfurylase/luciferase reaction is used to generate light for detection. In some embodiments, either or both the sulfurylase and luciferase are immobilized on one or more mobile solid supports disposed at each reaction site.

The present invention thus permits PPi release to be detected during the polymerase reaction giving a real-time signal. The sequencing reactions may be continuously monitored in real-time. A procedure for rapid detection of PPi release is thus enabled by the present invention. The reactions have been estimated to take place in less than 2 seconds (Nyren and Lundin, supra). The rate limiting step is the conversion of PPi to ATP by ATP sulfurylase, while the luciferase reaction is fast and has been estimated to take less than 0.2 seconds. Incorporation rates for polymerases have also been estimated by various methods and it has been found, for example, that in the case of Klenow polymerase, complete incorporation of one base may take less than 0.5 seconds. Thus, the estimated total time for incorporation of one base and detection by this enzymatic assay is approximately 3 seconds. It will be seen therefore that very fast reaction times are possible, enabling real-time detection. The reaction times could further be decreased by using a more thermostable luciferase.

For most applications it is desirable to use reagents free of contaminants like ATP and PPi. These contaminants may be removed by flowing the reagents through a pre-column containing apyrase and/-or pyrophosphatase bound to resin. Alternatively, the apyrase or pyrophosphatase can be bound to magnetic beads and used to remove contaminating ATP and PPi present in the reagents. In addition it is desirable to wash away diffusible sequencing reagents, e.g., unincorporated dNTPs, with a wash buffer. Any wash buffer used in pyrophosphate sequencing can be used.

In some embodiments, the concentration of reactants in the sequencing reaction include 1 pmol DNA, 3 pmol polymerase, 40 pmol dNTP in 0.2 ml buffer. See Ronaghi, et al., Anal. Biochem. 242: 84-89 (1996).

The sequencing reaction can be performed with each of four predetermined nucleotides, if desired. A “complete” cycle generally includes sequentially administering sequencing reagents for each of the nucleotides dATP, dGTP, dCTP and dTTP (or dUTP), in a predetermined order. Unincorporated dNTPs are washed away between each of the nucleotide additions. Alternatively, unincorporated dNTPs are degraded by apyrase (see below). The cycle is repeated as desired until the desired amount of sequence of the sequence product is obtained. In some embodiments, about 10-1000, 10-100, 10-75, 20-50, or about 30 nucleotides of sequence information is obtained from extension of one annealed sequencing primer.

In some embodiments, the nucleotide is modified to contain a disulfide-derivative of a hapten such as biotin. The addition of the modified nucleotide to the nascent primer annealed to the anchored substrate is analyzed by a post-polymerization step that includes i) sequentially binding of, in the example where the modification is a biotin, an avidin- or streptavidin-conjugated moiety linked to an enzyme molecule, ii) the washing away of excess avidin- or streptavidin-linked enzyme, iii) the flow of a suitable enzyme substrate under conditions amenable to enzyme activity, and iv) the detection of enzyme substrate reaction product or products. The hapten is removed in this embodiment through the addition of a reducing agent. Such methods enable a nucleotide to be identified in a given target position, and the DNA to be sequenced simply and rapidly while avoiding the need for electrophoresis and the use of potentially dangerous radiolabels.

A preferred enzyme for detecting the hapten is horse-radish peroxidase. If desired, a wash buffer, can be used between the addition of various reactants herein. Apyrase can be used to remove unreacted dNTP used to extend the sequencing primer. The wash buffer can optionally include apyrase.

Example haptens, e.g., biotin, digoxygenin, the fluorescent dye molecules cy3 and cy5, and fluorescein, are incorporated at various efficiencies into extended DNA molecules. The attachment of the hapten can occur through linkages via the sugar, the base, and via the phosphate moiety on the nucleotide. Example means for signal amplification include fluorescent, electrochemical and enzymatic. In a preferred embodiment using enzymatic amplification, the enzyme, e.g. alkaline phosphatase (AP), horse-radish peroxidase (HRP), beta-galactosidase, luciferase, can include those for which light-generating substrates are known, and the means for detection of these light-generating (chemiluminescent) substrates can include a CCD camera.

In a preferred mode, the modified base is added, detection occurs, and the hapten-conjugated moiety is removed or inactivated by use of either a cleaving or inactivating agent. For example, if the cleavable-linker is a disulfide, then the cleaving agent can be a reducing agent, for example dithiothreitol (DTT), beta-mercaptoethanol, etc. Other embodiments of inactivation include heat, cold, chemical denaturants, surfactants, hydrophobic reagents, and suicide inhibitors.

Luciferase can hydrolyze dATP directly with concomitant release of a photon. This results in a false positive signal because the hydrolysis occurs independent of incorporation of the dATP into the extended sequencing primer. To avoid this problem, a dATP analog can be used which is incorporated into DNA, i.e., it is a substrate for a DNA polymerase, but is not a substrate for luciferase. One such analog is α-thio-dATP. Thus, use of α-thio-dATP avoids the spurious photon generation that can occur when dATP is hydrolyzed without being incorporated into a growing nucleic acid chain.

Typically, the PPi-based detection is calibrated by the measurement of the light released following the addition of control nucleotides to the sequencing reaction mixture immediately after the addition of the sequencing primer. This allows for normalization of the reaction conditions. Incorporation of two or more identical nucleotides in succession is revealed by a corresponding increase in the amount of light released. Thus, a two-fold increase in released light relative to control nucleotides reveals the incorporation of two successive dNTPs into the extended primer.

If desired, apyrase may be “washed” or “flowed” over the surface of the solid support so as to facilitate the degradation of any remaining, non-incorporated dNTPs within the sequencing reaction mixture. Apyrase also degrades the generated ATP and hence “turns off” the light generated from the reaction. Upon treatment with apyrase, any remaining reactants are washed away in preparation for the following dNTP incubation and photon detection steps. Alternatively, the apyrase may be bound to the solid or mobile solid support.

Double Ended Sequencing

In a preferred embodiment we provide a method for sequencing from both ends of a nucleic acid template. Traditionally, the sequencing of two ends of a double stranded DNA molecule would require at the very least the hybridization of primer, sequencing of one end, hybridization of a second primer, and sequencing of the other end. The alternative method is to separate the individual strands of the double stranded nucleic acid and individually sequence each strand. The present invention provides a third alternative that is more rapid and less labor intensive than the first two methods.

The present invention provides for a method of sequential sequencing of nucleic acids from multiple primers. References to DNA sequencing in this application are directed to sequencing using a polymerase wherein the sequence is determined as the nucleotide triphosphate (NTP) is incorporated into the growing chain of a sequencing primer. One example of this type of sequencing is the pyro-sequencing detection pyrophosphate method (see, e.g., U.S. Pat. Nos. 6,274,320, 6258,568 and 6,210,891, each of which is incorporated in total herein by reference.).

In one embodiment, the present invention provides for a method for sequencing two ends of a template double stranded nucleic acid. The double stranded DNA is comprised of two single stranded DNA; referred to herein as a first single stranded DNA and a second single stranded DNA. A first primer is hybridized to the first single stranded DNA and a second primer is hybridized to the second single stranded DNA. The first primer is unprotected while the second primer is protected. “Protection” and “protected” are defined in this disclosure as being the addition of a chemical group to reactive sites on the primer that prevents a primer from polymerization by DNA polymerase. Further, the addition of such chemical protecting groups should be reversible so that after reversion, the now deprotected primer is once again able to serve as a sequencing primer. The nucleic acid sequence is determined in one direction (e.g., from one end of the template) by elongating the first primer with DNA polymerase using conventional methods such as pyrophosphate sequencing. The second primer is then deprotected, and the sequence is determined by elongating the second primer in the other direction (e.g., from the other end of the template) using DNA polymerase and conventional methods such as pyrophosphate sequencing. The sequences of the first and second primers are specifically designed to hybridize to the two ends of the double stranded DNA or at any location along the template in this method.

In another embodiment, the present invention provides for a method of sequencing a nucleic acid from multiple primers. In this method a number of sequencing primers are hybridized to the template nucleic acid to be sequenced. All the sequencing primers are reversibly protected except for one. A protected primer is an oligonucleotide primer that cannot be extended with polymerase and dNTPs which are commonly used in DNA sequencing reactions. A reversibly protected primer is a protected primer which can be deprotected. All protected primers referred to in this invention are reversibly protected. After deprotection, a reversibly protected primer functions as a normal sequencing primer and is capable of participating in a normal sequencing reaction.

The present invention provides for a method of sequential sequencing a nucleic acid from multiple primers. The method comprises the following steps: First, one or more template nucleic acids to be sequenced are provided. Second, a plurality of sequencing primers are hybridized to the template nucleic acid or acids. The number of sequencing primers may be represented by the number n where n can be any positive number greater than 1. That number may be, for example, 2, 3, 4, 5, 6, 7, 8, 9, 10 or greater. Of the primers, n-1 number may be protected by a protection group. So, for example, if n is 2, 3, 4, 5, 6, 7, 8, 9 or 10, n-1 would be 1, 2, 3, 4, 5, 6, 7, 8, 9 respectively. The remaining primer (e.g., n number primers—(n-1) number of protected primers=one remaining primer) is unprotected. Third, the unprotected primer is extended and the template DNA sequence is determined by conventional methods such as, for example, pyrophosphate sequencing. Fourth, after the sequencing of the first primer, one of the remaining protected primers is unprotected. Fifth, unprotected primer is extended and the template DNA sequence is determined by conventional methods such as, for example, pyrophosphate sequencing. Optionally, the method may be repeated until sequencing is performed on all the protected primers.

In another aspect, the present invention includes a method of sequential sequencing of a nucleic acid comprising the steps of: (a) hybridizing 2 or more sequencing primers to the nucleic acid wherein all the primers except for one are reversibly protected; (b) determining a sequence of one strand of the nucleic acid by polymerase elongation from the unprotected primer; (c) deprotecting one of the reversibly protected primers into an unprotected primer; (d) repeating steps (b) and (c) until all the reversibly protected primers are deprotected and used for determining a sequence. In one embodiment, this method comprises one additional step between steps (b) and (c), i.e., the step of terminating the elongation of the unprotected primer by contacting the unprotected primer with DNA polymerase and one or more of a nucleotide triphosphate or a dideoxy nucleotide triphosphate. In yet another embodiment, this method further comprises an additional step between said step (b) and (c), i.e., terminating the elongation of the unprotected primer by contacting the unprotected primer with DNA polymerase and a dideoxy nucleotide triphosphate from ddATP, ddTTP, ddCTP, ddGTP or a combination thereof.

In another aspect, this invention includes a method of sequencing a nucleic acid comprising: (a) hybridizing a first unprotected primer to a first strand of the nucleic acid; (b) hybridizing a second protected primer to a second strand; (c) exposing the first and second strands to polymerase, such that the first unprotected primer is extended along the first strand; (d) completing the extension of the first sequencing primer; (e) deprotecting the second sequencing primer; and (f) exposing the first and second strands to polymerase so that the second sequencing primer is extended along the second strand. In a preferred embodiment, completing comprises capping or terminating the elongation.

In another embodiment, the present invention provides for a method for sequencing two ends of a template double stranded nucleic acid that comprises a first and a second single stranded DNA. In this embodiment, a first primer is hybridized to the first single stranded DNA and a second primer is hybridized to the second single stranded DNA in the same step. The first primer is unprotected while the second primer is protected.

Following hybridization, the nucleic acid sequence is determined in one direction (e.g., from one end of the template) by elongating the first primer with DNA polymerase using conventional methods such as pyrophosphate sequencing. In a preferred embodiment, the polymerase is devoid of 3′ to 5′ exonuclease activity. The second primer is then deprotected, and its sequence is determined by elongating the second primer in the other direction (e.g., from the other end of the template) with DNA polymerase using conventional methods such as pyrophosphate sequencing. As described earlier, the sequences of the first primer and the second primer are designed to hybridize to the two ends of the double stranded DNA or at any location along the template. This technique is especially useful for the sequencing of many template DNAs that contain unique sequencing primer hybridization sites on its two ends. For example, many cloning vectors provide unique sequencing primer hybridization sites flanking the insert site to facilitate subsequent sequencing of any cloned sequence (e.g., Bluescript, Stratagene, La Jolla, Calif.).

One benefit of this method of the present invention is that both primers may be hybridized in a single step. The benefits of this and other methods are especially useful in parallel sequencing systems where hybridizations are more involved than normal. Examples of parallel sequencing systems are disclosed in copending U.S. patent application Ser. No. 10/104,280, the disclosure of which is incorporated in total herein.

The oligonucleotide primers of the present invention may be synthesized by conventional technology, e.g., with a commercial oligonucleotide synthesizer and/or by ligating together subfragments that have been so synthesized.

In another embodiment of the invention, the length of the double stranded target nucleic acid may be determined. Methods of determining the length of a double stranded nucleic acid are known in the art. The length determination may be performed before or after the nucleic acid is sequenced. Known methods of nucleic acid molecule length determination include gel electrophoresis, pulsed field gel electrophoresis, mass spectroscopy and the like. Since a blunt ended double stranded nucleic acid is comprised of two single strands of identical lengths, the determination of the length of one strand of a nucleic acid is sufficient to determine the length of the corresponding double strand.

The sequence reaction according to the present invention also allows a determination of the template nucleic acid length. First, a complete sequence from one end of the nucleic acid to another end will allow the length to be determined. Second, the sequence determination of the two ends may overlap in the middle allowing the two sequences to be linked. The complete sequence may be determined and the length may be revealed. For example, if the template is 100 bps long, sequencing from one end may determine bases 1 to 75; sequencing from the other end may determine bases 25 to 100; there is thus a 51 base overlap in the middle from base 25 to base 75; and from this information, the complete sequence from 1 to 100 may be determined and the length, of 100 bases, may be revealed by the complete sequence.

Another method of the present invention is directed to a method comprising the following steps. First a plurality of sequencing primers, each with a different sequence, is hybridized to a DNA to be sequenced. The number of sequencing primers may be any value greater than one such as, for example, 2, 3, 4, 5, 6, 7, 8, 9, 10 or more. All of these primers are reversibly protected except for one. The one unprotected primer is elongated in a sequencing reaction and a sequence is determined. Usually, when a primer is completely elongated, it cannot extend and will not affect subsequent sequencing from another primer. If desired, the sequenced primer may be terminated using excess polymerase and dNTP or using ddNTPs. If a termination step is taken, the termination reagents (dNTPs and ddNTPs) should be removed after the step. Then, one of the reversibly protected primers is unprotected and sequencing from the second primer proceeds. The steps of deprotecting a primer, sequencing from the deprotected primer, and optionally, terminating sequencing from the primer is repeated until all the protected primers are unprotected and used in sequencing.

The reversibly protected primers should be protected with different chemical groups. By choosing the appropriate method of deprotection, one primer may be deprotected without affecting the protection groups of the other primers. In a preferred embodiment, the protection group is PO₄. That is, the second primer is protected by PO₄ and deprotection is accomplished by T4 polynucleotide kinase (utilizing its 3′-phosphatase activity). In another preferred embodiment, the protection is a thio group or a phosphorothiol group.

The template nucleic acid may be a DNA, RNA, or peptide nucleic acid (PNA). While DNA is the preferred template, RNA and PNA may be converted to DNA by known techniques such as random primed PCR, reverse transcription, RT-PCR or a combination of these techniques. Further, the methods of the invention are useful for sequencing nucleic acids of unknown and known sequence. The sequencing of nucleic acid of known sequence would be useful, for example, for confirming the sequence of synthesized DNA or for confirming the identity of suspected pathogen with a known nucleic acid sequence. The nucleic acids may be a mixture of more than one population of nucleic acids. It is known that a sequencing primer with sufficient specificity (e.g., 20 bases, 25 bases, 30 bases, 35 bases, 40 bases, 45 bases, or 50 bases) may be used to sequence a subset of sequences in a long nucleic acid or in a population of unrelated nucleic acids. Thus, for example, the template may be one sequence of 10 Kb or ten sequences of 1 Kb each. In a preferred embodiment, the template DNA is between 50 bp to 700 bp in length. The DNA can be single stranded or double stranded.

In the case where the template nucleic acid is single stranded, a number of primers may be hybridized to the template nucleic acid as shown below:
5′—primer 4—3′ 5′-primer 3—3′ 5′-primer2-3′ 5′-primer 1-3′
3′ - - - template nucleic acid - - - 5′

In this case, it is preferred that the initial unprotected primer would be the primer that hybridizes at the most 5′ end of the template. See primer 1 in the above illustration. In this orientation, the elongation of primer 1 would not displace (by strand displacement) primer 2, 3, or 4. When sequencing from primer 1 is finished, primer 2 can be unprotected and nucleic acid sequencing can commence. The sequencing from primer 2 may displace primer 1 or the elongated version of primer one but would have no effect on the remaining protected primers (primers 3 and 4). Using this order, each primer may be used sequentially and a sequencing reaction from one primer would not affect the sequencing from a subsequent primer.

One feature of the invention is the ability to use multiple sequencing primers on one or more nucleic acids and the ability to sequence from multiple primers using only one hybridization step. In the hybridization step, all the sequencing primers (e.g., the n number of sequencing primers) may be hybridized to the template nucleic acid(s) at the same time. In conventional sequencing, usually one hybridization step is required for sequencing from one primer. One feature of the invention is that the sequencing from n primers (as defined above) may be performed by a single hybridization step. This effectively eliminates n-1 hybridization step.

In a preferred embodiment, the sequences of the n number of primers are sufficiently different that the primers do not cross hybridize or self-hybridize. Cross hybridization refers to the hybridization of one primer to another primer because of sequence complementarity. One form of cross hybridization is commonly referred to as a “primer dimer.” In the case of a primer dimer, the 3′ ends of two primers are complementary and form a structure that when elongated, is approximately the sum of the length of the two primers. Self-hybridization refers to the situation where the 5′ end of a primer is complementary to the 3′ end of the primer. In that case, the primer has a tendency to self hybridize to form a hairpin-like structure.

A primer can interact or become associated specifically with the template molecule. By the terms “interact” or “associate”, it is meant herein that two substances or compounds (e.g., primer and template; chemical moiety and nucleotide) are bound (e.g., attached, bound, hybridized, joined, annealed, covalently linked, or otherwise associated) to one another sufficiently that the intended assay can be conducted. By the terms “specific” or “specifically”, it is meant herein that two components bind selectively to each other. The parameters required to achieve specific interactions can be determined routinely, e.g., using conventional methods in the art.

To gain more sensitivity or to help in the analysis of complex mixtures, the protected primers can be modified (e.g., derivatized) with chemical moieties designed to give clear unique signals. For example, each protected primer can be derivatized with a different natural or synthetic amino acid attached through an amide bond to the oligonucleotide strand at one or more positions along the hybridizing portion of the strand. The chemical modification can be detected, of course, either after having been cleaved from the target nucleic acid, or while in association with the target nucleic acid. By allowing each protected target nucleic acid to be identified in a distinguishable manner, it is possible to assay (e.g., to screen) for a large number of different target nucleic acids in a single assay. Many such assays can be performed rapidly and easily. Such an assay or set of assays can be conducted, therefore, with high throughput efficiency as defined herein.

In the methods of the invention, after a first primer is elongated and the sequence of the template DNA is determined, a second primer is deprotected and sequenced. There is no interference between the sequencing reaction of the first primer with the sequencing reaction of the second, now unprotected, primer because the first primer is completely elongated or terminated. Because the first primer is completely elongated, the sequencing from the second primer, using conventional methods such a pyrophosphate sequencing, will not be affected by the presence of the elongated first primer. The invention also provides a method of reducing any possible signal contamination from the first primer. Signal contamination refers to the incidences where the first primer is not completely elongated. In that case, the first primer will continue to elongate when a subsequent primer is deprotected and elongated. The elongation of both the first and second primers may interfere with the determination of DNA sequence.

In a preferred embodiment, the sequencing reaction (e.g., the chain elongation reaction) from one primer is first terminated or completed before a sequencing reaction is started on a second primer. A chain elongation reaction of DNA can be terminated by contacting the template DNA with DNA polymerase and dideoxy nucleotide triphosphates (ddNTPs) such as ddATP, ddTTP, ddGTP and ddCTP. Following termination, the dideoxy nucleotide triphosphates may be removed by washing the reaction with a solution without ddNTPs. A second method of preventing further elongation of a primer is to add nucleotide triphosphates (dNTPs such as dATP, dTTP, dGTP and dCTP) and DNA polymerase to a reaction to completely extend any primer that is not completely extended. Following complete extension, the dNTPs and the polymerases are removed before the next primer is deprotected. By completing or terminating one primer before deprotecting another primer, the signal to noise ratio of the sequencing reaction (e.g., pyrophosphate sequencing) can be improved.

The steps of (a) optionally terminating or completing the sequencing, (b) deprotecting a new primer, and (c) sequencing from the deprotected primer may be repeated until a sequence is determined from the elongation of each primer. In this method, the hybridization step comprises “n” number of primers and one unprotected primer. The unprotected primer is sequenced first and the steps of (a), (b) and (c) above may be repeated.

In a preferred embodiment, pyrophosphate sequencing is used for all sequencing conducted in accordance with the method of the present invention.

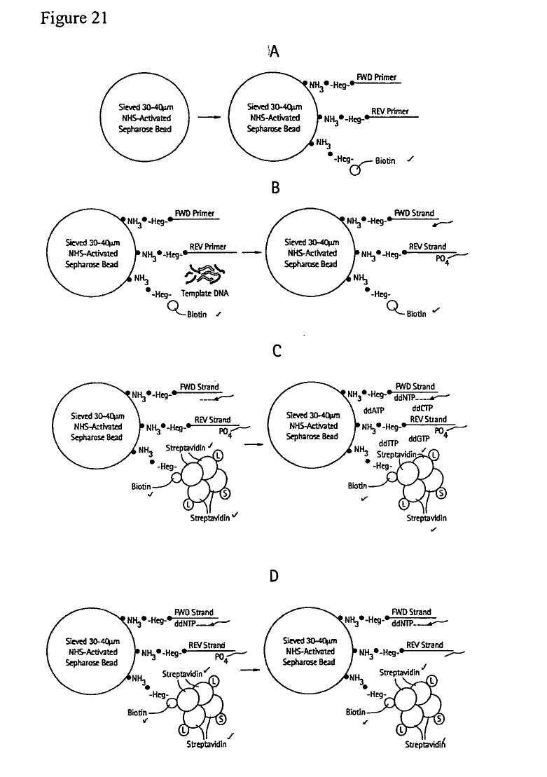

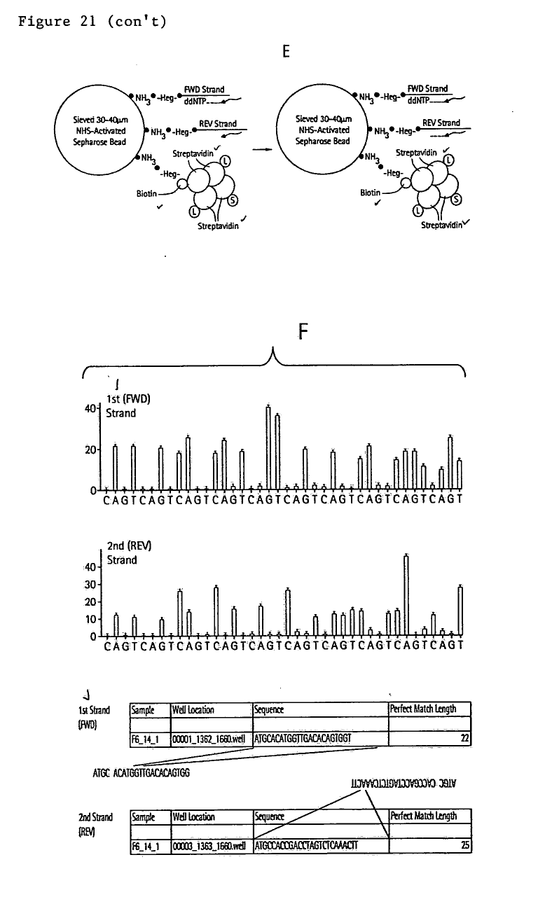

In another preferred embodiment, the double ended sequencing is performed according to the process outlined in FIG. 21. This process may be divided into six steps: (1) creation of a capture bead (FIG. 21); (2) drive to bead (DTB) PCR amplification (FIG. 21); (3) SL reporter system preparation (FIG. 10C); (4) sequencing of the first strand (FIG. 21); (5) preparation of the second strand (FIG. 21); and (6) analysis of each strand (FIG. 21). This exemplary process is outlined below.

In step 1, an N-hydroxysuccinimide (NHS)-activated capture bead (e.g., Amersham Biosciences, Piscataway, N.J.) is coupled to both a forward primer and a reverse primer. NHS coupling forms a chemically stable amide bond with ligands containing primary amino groups. The capture bead is also coupled to biotin (FIG. 21). The beads (i.e., solid nucleic acid capturing supports) used herein may be of any convenient size and fabricated from any number of known materials. Example of such materials include: inorganics, natural polymers, and synthetic polymers. Specific examples of these materials include: cellulose, cellulose derivatives, acrylic resins, glass; silica gels, polystyrene, gelatin, polyvinyl pyrrolidone, co-polymers of vinyl and acrylamide, polystyrene cross-linked with divinylbenzene or the like (see, Merrifield Biochemistry 1964, 3, 1385-1390), polyacrylamides, latex gels, polystyrene, dextran, rubber, silicon, plastics, nitrocellulose, celluloses, natural sponges, silica gels, glass, metals plastic, cellulose, cross-linked dextrans (e.g., Sephadex™) and agarose gel (Sepharose™) and solid phase supports known to those of skill in the art. In a preferred embodiment, the capture beads are Sepharose beads approximately 25 to 40 μM in diameter.

In step 2, template DNA which has hybridized to the forward and reverse primers is added, and the DNA is amplified through a PCR amplification strategy (FIG. 21). In one embodiment, the DNA is amplified by Emulsion Polymerase Chain Reaction, Drive to Bead Polymerase Chain Reaction, Rolling Circle Amplification or Loop-mediated Isothermal Amplification. In step 3, streptavidin is added followed by the addition of sulfurylase and luciferase which are coupled to the streptavidin (FIG. 21). The addition of auxiliary enzymes during a sequencing method has been disclosed in U.S. Ser. No. 10/104,280 and U.S. Ser. No. 10/127,906, which are incorporated herein in their entireties by reference. In one embodiment, the template DNA has a DNA adaptor ligated to both the 5′ and 3′ end. In a preferred embodiment, the DNA is coupled to the DNA capture bead by hybridization of one of the DNA adaptors to a complimentary sequence on the DNA capture bead.

In the first step, single stranded nucleic acid template to be amplified is attached to a capture bead. The nucleic acid template may be attached to the capture bead in any manner known in the art. Numerous methods exist in the art for attaching the DNA to a microscopic bead. Covalent chemical attachment of the DNA to the bead can be accomplished by using standard coupling agents, such as water-soluble carbodiimide, to link the 5′-phosphate on the DNA to amine-coated microspheres through a phosphoamidate bond. Another alternative is to first couple specific oligonucleotide linkers to the bead using similar chemistry, and to then use DNA ligase to link the DNA to the linker on the bead. Other linkage chemistries include the use of N-hydroxysuccinamide (NHS) and its derivatives, to join the oligonucleotide to the beads. In such a method, one end of the oligonucleotide may contain a reactive group (such as an amide group) which forms a covalent bond with the solid support, while the other end of the linker contains another reactive group which can bond with the oligonucleotide to be immobilized. In a preferred embodiment, the oligonucleotide is bound to the DNA capture bead by covalent linkage. However, non-covalent linkages, such as chelation or antigen-antibody complexes, may be used to join the oligonucleotide to the bead.

Oligonucleotide linkers can be employed which specifically hybridize to unique sequences at the end of the DNA fragment, such as the overlapping end from a restriction enzyme site or the “sticky ends” of bacteriophage lambda based cloning vectors, but blunt-end ligations can also be used beneficially. These methods are described in detail in U.S. Pat. No. 5,674,743, the disclosure of which is incorporated in toto herein. It is preferred that any method used to immobilize the beads will continue to bind the immobilized oligonucleotide throughout the steps in the methods of the invention. In a preferred embodiment, the oligonucleotide is bound to the DNA capture bead by covalent linkage. However, non-covalent linkages, such as chelation or antigen-antibody complexes, may be used to join the oligonucleotide to the bead.

In step 4, the first strand of DNA is sequenced by depositing the capture beads onto a PicoTiter plate (PTP), and sequencing by a method known to one of ordinary skill in the art (e.g., pyrophosphate sequencing) (FIG. 21). Following sequencing, a mixture of dNTPs and ddNTPs are added in order to “cap” or terminate the sequencing process (FIG. 21). In step 5, the second strand of nucleic acid is prepared by adding apyrase to remove the ddNTPs and polynucleotide kinase (PNK) to remove the 3′ phosphate group from the blocked primer strand (FIG. 21). Polymerase is then added to prime the second strand followed by sequencing of the second strand according to a standard method known to one of ordinary skill in the art (FIG. 21). In step 7, the sequence of the both the first and second strand is analyzed such that a contiguous DNA sequence is determined.

The methods disclosed may be use for: (1) cell population sequencing wherein 1, 2 or more genes from large numbers (100,000+) of individual cells may be sequenced concurrently, a truly revolutionary approach to study autoimmune disorders and immunity to tumors; (2) a method for conducting genome-wide methylation occurring as the result of disease and/or aging may be accessed; and (3) complex-sample sequencing wherein fragments of genetic material from a mixture of, for example, microorganisms from blood, air, water, food, or other sources may be prepared and sequenced together, and wherein the individual members of the sample mixture may be identified by computational matching to larger sequence databases.

### 5. EXAMPLES

The examples are presented in order to more fully illustrate the preferred embodiments of the invention. These examples should in no way be construed as limiting the scope of the invention, as encompassed by the appended claims.

### Example 1

### Principles of Sequence-Based Karyotyping

The sensitivity and specificity of Sequence-Based Karyotyping in detecting genome-wide changes was expected to depend on several factors. The breadth of the region of amplification or deletion and the magnitude of the change in copy number of a given genomic event will directly effect the detection of the change.

Analysis of Whole Chromosomes

We attempted to determine whether any loss or gain of chromosomal content was present in DiFi cells that were detectable using Sequence-Based Karyotyping relative to the published findings by digital karyotyping. Briefly, all the DNA sequences obtained were mapped to a genomic scaffold. Sequences that did not map to the genome, either due to incompleteness of the genomic scaffold or issues of sequencing quality, were removed from consideration. Filtering was also performed to remove DNA sequences which mapped to multiple genomic locations (within repeated sequences). Counts of the resulting number of unique hits to each chromosome were tabulated for both the test DiFi sample and the reference GM12911 sample. For each chromosome, the ratio of the number of unique hits in the DiFi sample to the corresponding number of hits in the GM12911 sample was computed, providing a raw ratio of measured chromosomal content on a per chromosome basis. The raw ratios were further normalized to account for any difference in the amount of actual sequencing performed for the two samples; specifically, the ratio of the total number of unique hits to the autosomal chromosomes in the DiFi and GM12911 samples was used as a multiplicative normalization factor to convert the raw chromosomal content ratios into normalized ratios. Each of these normalized ratios, for the autosomal chromosomes, was then multiplied by 2 to provide a normalized, measured chromosomal content for a diploid genome. Data for the Y chromosome was removed as the DiFi sample was from a female and the GM12911 sample was from a male. No multiplication by 2 was performed for the X chromosome since the female DiFi sample was already expected to have twice the X content as the male GM12911 sample. The resulting diploid-based chromosomal content estimates were compared with those of Wang et al (17), as shown in FIG. 1, and found to have very high correlation (R²=0.97),validating our estimates of aneuploidy. In this figure each point represents a chromosome with a content computed in terms of a diploid genome. A “Chromosome Content” of 2.0 represents a chromosome without amplification or deletion. Larger values imply the existence of regions of amplification and smaller values imply regions of deletion. Extremely low values (less than 1.5) are assumed to represent the loss of a chromosome, extremely high values (greater than 3.0) are assumed to represent the gain of a chromosome. The figure contains only 23 data points because the DiFi cells were of female origin and so there was no “Y” chromosomal content to plot.

Analysis of Amplifications

To identify amplifications, which typically involve regions much smaller than a chromosomal arm, analysis was performed as described below to identify fragments recovered more frequently than expected by chance and/or more frequently than karyotypically normal cells.

Wang et al (17) have previously reported gene amplifications on chromosomes 7, 13, and 20. The Sequence-Based Karyotyping method found, with statistical significance, the same amplifications on chromosomes 7, and found the two reported amplifications on 13 and 20, but without significance. However, the Sequence-Based Karyotyping method also found 4 putative amplifications not previously reported, ˜3.6 fold Chr10 55.73-56.35 MB, ˜4.6 fold Chr13 22.43-22.78 MB, ˜3.6 fold Chr14 23.68-24.41 MB, ˜3.6 fold Chr18 7.66-8.54 MB, and eight ˜4 fold amplification regions on chromosome 5.

Although there is the possibility that some of these amplifications are false positives, another possibility is that they were only discovered by Sequence-Based Karyotyping because it is implemented based on the sequencing of random fragments and, unlike Digital Karyotyping, is not biased in only being able to report data for sections of the genome adjacent to specific restriction enzyme sites.

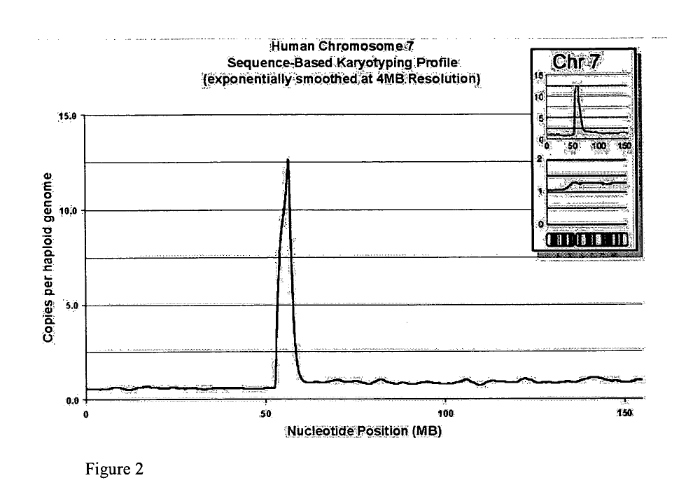

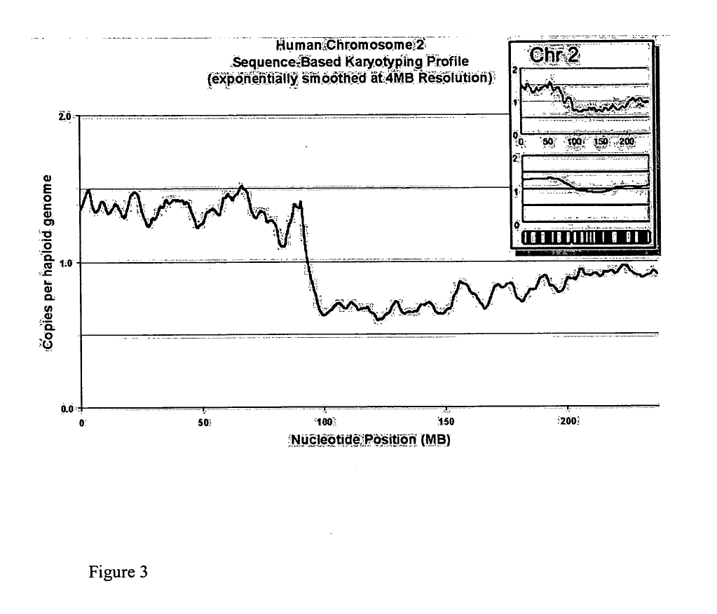

FIGS. 2 and 3 show more detailed resolution of the amplification on chromosome 7 and the overall chromosomal content on chromosome 2, respectively. Sequence-Based Karyotyping is capable of far greater resolution than the 4 Mb resolution used in these figures; however, this resolution was chosen in order to facilitate comparison with similar previously published data for Digital Karyotyping and CGH which was plotted at an approximate 4 Mb resolution. Qualitatively we see the shapes of the curves of Sequence-Based Karyotyping and Digital Karyotyping are similar. Both are able to detect the large amplification on Chromosome 7 that is not detected by CGH.

Analysis of Deletions

When a homozygous deletion occurs in a cancer cell, there are zero copies of the deleted sequences compared to two copies in normal cells. This difference is far less than that observed with amplifications, wherein 10-200 copies of the involved sequences are present in cancer cells compared to two copies in normal cells. Detection of homozygous deletions was therefore expected to be more difficult than the detection of amplifications.

We attempted to determine whether any deletions were present in DiFi cells that were detectable using Sequence-Based Karyotyping relative to the published findings by digital karyotyping. Two confirmed specific deletions for the DiFi cell line were published, one on chromosome 5 and the other on chromosome X. The chromosome 5 deletion is not found with significance, but the chromosome X deletion is found with high significance. Additional deletions on chromosomes 3, 9, 13, and another location on X were found by Sequence-Based Karyotyping.

### Example 2

### Materials and Methods for Sequence-Based Karyotyping

Sequence-Based Karyotyping was performed on DNA from the DiFi colorectal cancer cell line, and from lymphoblastoid cells of a normal individual (GM1291 1, obtained from Coriell Cell Repositories, NJ). Genomic DNA was isolated using DNeasy or QIAamp DNA blood kits (Qiagen, Chatsworth, Calif.) using the manufacturers' protocols.

Briefly, DNA is fragmented and size fractionated. Fragments within a several hundred basepair size range are ligated to proprietary adapters to generate templates. These templates are suitable for subsequent PCR and sequencing reactions using the sequencing methods described in this disclosure (454 Life Sciences technology). The adapted templates are amplified using a proprietary oil-water emulsion PCR system. The amplified DNA molecules are then immobilized onto proprietary microscopic beads and collected. The beads containing amplified DNA are subsequently segregated from non-DNA containing beads and used for sequencing. The DNA-containing beads are loaded into a glass fiber plate containing microwells. Individual sequencing reactions occur in the microwells. The DNA sequence of the individual templates is determined by repetitively flowing each individual nucleotide and indirectly monitoring the release of PPi as DNA synthesis off the template proceeds. Light emitted during these individual sequencing reactions is captured and computationally transformed into DNA sequence reads. The data are further computationally processed to yield high quality DNA sequences according to predetermined quality standards

Sequences were generated as follows: Male Normal (GM12911) sample: 354,451 total sequence reads (94.9 bp on average); Female Cancer (DiFi): 487,310 total sequence reads (97.1 bp on average)

All sequences were mapped to the Human Genome using the criteria of at least 95% identity over 90% of the read length. Any sequences that mapped to more than one position were discarded. This resulted in 125,684 Normal and 203,352 DiFi fragments uniquely mapped to the Genome.

### Example 3

### Data Analysis

Genomic sequences are analyzed for insertions, deletions, and aneuploidy by comparing fragments sequenced from a normal reference sample to fragments sequenced from an experimental sample. Reads from the normal reference genome may be generated at the same time as those for the experimental sample (to better account for date-specific facility affects) or a standard library of reads from a reference genome may be generated once and reused for multiple projects. Finally, a computational reference genome can be constructed by high density random sampling of the known genome and determining how many unique sequences there are within given sub-regions of each chromosome based on sequence reads of size commensurate with the average read length of the sequencing. Statistics from these computational methods can be combined with statistics from actually sampled normal samples to compute platform-specific irregularities in sequencing density that might otherwise be confused with actual differences if the theoretical computational database were directly compared against fragments from an experimental sample.

Fragments reads from both the normal reference (either sequenced, or computationally generated) and experimental, test samples are mapped, by sequence similarity, to a reference genome. The reference genomes used are divided into two populations of chromosomal sequence: that portion which is ordered and assembled and the rest (which may come from known chromosomes but for which the ordering and positioning of the genomic DNA is not well characterized or genomic DNA which is known to be from the genome but not associated with any particular chromosome). We refer to the ordered and assembled portion of the genome as the “known genome” and the rest as the “random genome.” In addition there is generally additional genomic information available for the genome of the Mitochondrion of the reference genome.

Reads which map to multiple locations on the genome are discarded. A read is considered to map to multiple locations if it maps to more than one location on the known genome or to a single location on the known genome and any location on the random genome or to any location on the associated mitochondrial genome. For assays concerning the mitochondrial genome itself, a read is considered to map to multiple locations if it maps to more than one location on the mitochondrial genome or to one location on the mitochondrial genome and to any other location on the known or random reference genome.

Discovery of deletions and increased copy of genomic regions is performed by considering each chromosome individually. Based on the desired ability to discover amplifications versus deletions, a critical “pooling” value is chosen. Higher pooling value are chosen to discover deletions and lower values are chosen to discover increased copy numbers. Given the pooling value, one divides each chromosome into consecutive regions such that each region contains a minimum of the pooling value of normal fragments that uniquely map within the so induced region. Given regions defined in this manner, one tabulates the number of uniquely mapping test fragments that map within the same regions. The resulting set of numbers are then analyzed according to a number of contingency table based methods. First, a contingency table with two rows can be constructed with one row corresponding to the reference sample and one row corresponding to the test sample. Each column of the table corresponds to the regions of the chromosome induced by the procedure involving the pooling value. A standard Chi-square analysis of the resulting contingency table can indicate whether there are any regions of significantly different copy number overall, independent of any affect of aneuploidy (which is automatically factored out by the Chi-square analysis).

For a contingency table with N columns, corresponding to N pooled regions of the genome, a series of (N-1) 2×2 contingency tables can also be constructed by picking a single column of interest and summing over all the other columns into a single marginalized value. We compute all such 2×2 tables and sort them from smallest p-value to largest, picking the table with the smallest value. If that value exceeds a multiple-testing corrected p-value (described below) then we choose the difference represented by that table as significant. The counts, contained in the significantly different column of data, are removed from the original table and now the original global table has N-1 columns and two rows. We proceed in this fashion, continuing to remove columns so long as the minimal p-value is below a multiple-test corrected p-value (described below). At that point, a set of zero or more columns, corresponding to regions of the genome, have been removed from the original table, and the relevant genes, regulatory regions, and other genomic features are determined by database lookup of genomic features that have been mapped to the reference scaffold in the regions corresponding to the removed columns. Relative amplification and deletions within these regions can be computed from the ratio of the number of uniquely mapped fragment counts in the corresponding genomic region between the reference and test samples (normalized by the amount of sequencing performed on the two samples). Additionally, relative amplifications and deletions may be computed by looking at the ratios of counts solely of the test sample itself in the region of interest to the test sample counts in immediately neighboring genomic regions (this may often give a more accurate estimate assuming the neighboring regions are not themselves unduly amplified or deleted).

This same procedure could be applied on a whole genome basis by simply combining all the chromosomes into a single contingency table, rather than by treating each chromosome separately. One could also divide the genome up according to regions of fixed size and perform the same analysis either on a per-chromosome basis or on the genome as a whole. The advantage of the above pooling method is that by choosing a sufficiently large pooling value (typically>=5), one can virtually guarantee that the assumptions of a Chi-square analysis will be met (namely that no fewer than 20% of the cells in the table have an expected value of less than 5 and that none have an expected value of 0). Whether pooling is used or not, if the Chi-square analysis assumptions have not been met, then one can merge adjacent cells of the table until the assumptions have been met, merging the coordinates of the corresponding regions of the genome.

One may additionally choose to bias the pooling so that one does not pool across contigs that have a large gap between them in the genome assembly, and instead place excess counts so that they occur in the last region of the last assembly contig, and only start creating new regions at the beginning of the next assembly contig. Another option is to pool based on aggregate genomic features of interest (such as the entire p region vs the entire q region of each chromosome) allowing one to decide if there is unusual distribution of hits relative to these features. In the extreme, one could make a contingency table of the entire genome, with one column per chromosome to identify chromosomes that are over or underrepresented in content at the entire chromosomal level. Ratios, on a per chromosomal basis, of the number of uniquely mapping fragments in the experimental sample to the number in the normal sample (corrected by the ratio of the total number of uniquely mapping sequences to the entire genome of the normal sample over the number in the experimental sample, to correct for differences in the amount of sequencing in the two samples), can be used to estimate rates of aneuploidy. Choosing larger pooling values has the affect of aggregating the genome into larger physical regions and smaller pooling values aggregates the genome into smaller regions. The larger the physical region, the more averaged out any given effect, especially deletions, will be. On the other hand, the larger the pooling value, the greater statistical certainty will be associated with an observed deletion in the experimental sample. Thus, there is a tension between observing deletions and having good statistical p values with those deletions. Pooling values we typically use are 5, 10, 20, and 40.

A multiple testing correction is applied given that multiple statistical tests are performed in order to avoid inflated rates of false positives. If the chromosomes are pooled and evaluated separately, one can decide on an overall false positive rate, p_{false}, a priori. For example, if one is studying just the autosomal chromosomes, one might choose p_{false}= 1/22 (˜0.0455), for female samples, one might choose p_{false}= 1/23 (˜0.0435), and for male samples, one might choose p_{false}= 1/24 (˜0.042), so that the number of false positive regions of difference does not exceed 1 given that 22, 23, or 24 chromosomes are going to be evaluated in the these cases, respectively. These values can be scaled by an arbitrary factor of ƒ(i.e., f/22, f/23, f/24) if a total of ƒ false positives are acceptable. Alternatively, traditional standard p-values of 0.001, 0.01, and 0.05 might be employed.

Each chromosome is separately evaluated in a series of at N-1 iterations of finding minimal p-score 2×2 chi-square tables (where N is different for each chromosome). On the i'th such iteration, there are potentially N-i total subsequent iterations that may be performed, and so a conservative p-value to use on the i'th iteration is
1-(1-p_{false})^{(1/(N-i))}

Rather than apportioning the same error to each chromosome, one might instead choose to apportion the error over the entire genome. Summing all the N regions induced over all the chromosomes one gets an overall N_{genome}. One can then formulate a desired false positive rate, as above, associated with this number of comparisons where the iteration count continues to increase (and does not restart with 1 as one goes from each chromosomal contingency table to the other). The same correction factor may be used when the entire genome is put into a single contingency table (in which case there are N_{genome} columns in that table). All of the above may also be performed with multiple test samples in separate rows of the contingency table against a single reference sample row, or even with test samples and no reference sample in order to find the relationship between different test samples.

### Example 4

### Preparation of DNA Sample For Sequence-Based Karyotyping

DNA Sample:

Step 1: DNase I Digestion

DNA was obtained and prepared to a concentration of 0.3 mg/ml in Tris-HCl (10 mM, pH 7-8). A total of 134 μl of DNA (15 μg) was needed for this preparation. It is recommended to not use DNA preparations diluted with buffers containing EDTA (i.e., TE, Tris/EDTA).

In a 0.2 ml tube, DNase I Buffer, comprising 50 μl Tris pH 7.5 (1M), 10 μl MnCl₂ (1M), 1 μl BSA (100 mg/ml), and 39 μl water was prepared.

In a separate 0.2 ml tube, 15 μl of DNase I Buffer and 1.5 μl of DNase I (1 U/ml) was added. The reaction tube was placed in a thermal cycler set to 15° C.

The 134 μl of DNA (0.3 mg/ml) was added to the DNase I reaction tube placed in the thermal cycler set at 15° C. The lid was closed and the sample was incubated for exactly 1 minute. Following incubation, 50 μl of 50 mM EDTA was added to stop the enzyme digestion.

The digested DNA was purified by using the QiaQuick PCR purification kit. The digestion reaction was then split into four aliquots, and four spin columns were used to purify each aliquot (37.5 μl per spin column). Each column was eluted with 30 μl elution buffer (EB) according to the manufacturer's protocol. The eluates were then combined to generate a final reaction volume of 120 μl.

One 3 μl aliquot of the digestion reaction was saved for analysis using a BioAnalzyer DNA 1000 LabChip.

Step 2: Pfu Polishing

The following Pfu polishing protocol was used.
1. In a 0.2 ml tube, 115 μl purified, DNase I-digested DNA fragments, 15 μl 10× Cloned Pfu buffer, 5 μl dNTPs (10 mM), and 15 μl cloned Pfu DNA polymerase (2.5 U/μl) were added in order.
2. The polishing reaction components were mixed well and incubated at 72° C. for 30 minutes.
3. Following incubation, the reaction tube was removed and placed on ice for 2 minutes.
4. The polishing reaction mixture was then split into four aliquots and purified using QiaQuick PCR purification columns (37.5 μL on each column). Each column was eluted with 30 μl buffer EB according to the manufacturer's protocol. The eluates were then combined to generate a final reaction volume of 120 μL.
5. One 3 μl aliquot of the final polishing reaction was saved for analysis using a BioAnalzyer DNA 1000 LabChip.

Step 3: Ligation of Universal Adaptors to Fragmented DNA Library

Each Universal Adaptor is prepared by annealing, in a single tube, the two single-stranded complementary DNA oligonucleotides (i.e., one oligo containing the sense sequence and the second oligo containing the antisense sequence). The following ligation protocol was used.
6. In a 0.2 ml tube, 39 μl nH₂O (molecular biology grade water), 25 μl digested, polished DNA Library, 100 μl 2× Quick Ligase Reaction Buffer, 20 μl MMP1 (10 pm/μl) adaptor set, 100:1 ratio, and 16 μl Quick Ligase were added in order. The ligation reaction was mixed well and incubated at RT for 20 minutes.
7. The ligation reaction was then removed and a 10-μl aliquot of the ligation reaction was purified for use on the BioAnalyzer. A single spin column from the Qiagen MinElute kit was used. The column was eluted with 10 μl EB according to the procedure per manufacturers' protocol. A 1-μl aliquot of the purified ligation reaction was loaded using a BioAnalyzer DNA 1000 LabChip. This purification step is recommended as the unpurified ligation reaction contains high amounts of salt and PEG that will inhibit the sample from running properly on the BioAnalyzer.
8. The remainder of the ligation reaction (190 μL) was used for gel isolation in Step 4.

Step 3a: Microcon Filtration and Adaptor Construction. Total preparation time was approximately 25 min.

The Universal Adaptor ligation reaction requires a 100-fold excess of adaptors. To aid in the removal of these excess adaptors, the double-stranded gDNA library is filtered through a Microcon YM-100 filter device. Microcon YM-100 membranes can be used to remove double stranded DNA smaller than 125 bp. Therefore, unbound adaptors (44 bp), as well as adaptor dimers (88 bp) can be removed from the ligated gDNA library population. The following filtration protocol was used:
1. The 190 μL of the ligation reaction from Step 4 was applied into an assembled Microcon YM-100 device.
2. The device was placed in a centrifuge and spun at 5000×g for approximately 6 minutes, or until membrane was almost dry.
3. To wash, 200 μl of 1× TE was added.
4. Sample was spun at 5000×g for an additional 9 minutes, or until membrane was almost dry.
5. To recover, the reservoir was inserted into a new vial and spun at 3000×g for 3 minutes. The reservoir was discarded. The recovered volume was approximately 10 μl. Next, 80 μl TE was added.

The Adaptors (A and B) were HPLC-purified and modified with phosphorothioate linkages prior to use. For Adaptor “A” (10 μM), 10 μl of 100 μM Adaptor A (44 bp, sense) was mixed with 10 μl of 100 μM Adaptor A (40 bp, antisense), and 30 μl of 1× Annealing Buffer (V_{f}=50 μl) were mixed. The primers were annealed using the ANNEAL program on the Sample Prep Labthermal cycler (see below). For Adaptor “B” (10 μM), 10 μl of 100 μM Adaptor B (40 bp, sense) was mixed with 10 μl of 100 μM Adaptor B (44 bp, antisense), and 30 μl of 1× Annealing Buffer (V_{f}=50 μl). The primers were annealed using the ANNEAL program on the Sample Prep Lab thermal cycler. Adaptor sets could be stored at −20° C. until use.

ANNEAL-A program for primer annealing:
1. Incubate at 95° C., 1 min;
2. Decrease temperature to 15° C., at 0.1° C./sec; and
3. Hold at 15° C.

Step 4: Gel Electrophoresis and Extraction of Adapted DNA Library

Adaptor dimers will migrate at 88 bp and adaptors unligated will migrate at 44 bp. Therefore, genomic DNA libraries in size ranges >200 bp can be physically isolated from the agarose gel and purified using standard gel extraction techniques. Gel isolation of the adapted DNA library will result in the recovery of a library population in a size range that is ≧200 bp (size range of library can be varied depending on application). The following electrophoresis and extraction protocol was used.
1. A 2% agarose gel was prepared.
2. 10 μl of 10× Ready-Load Dye was added to the remaining 90 μl of the DNA ligation mixture.
3. The dye/ligation reaction mixture was loaded into the gel using four adjacent lanes (25 μl per lane).
4. 10 μl of the100 bp ladder (0.1 μg/μl) was loaded two lanes away from ligation reaction lanes.
5. The gel was run at 100V for 3 hours.
6. When the gel run was complete, DNA bands were visualized using a hand-held long-wave UV light. Using a sterile, single-use scalpel, the fragment sizes of 200-400 bp were cut out from the agarose gel. Using this approach, libraries with any size range can be isolated.
7. The DNA embedded in the agarose gel was isolated using a Qiagen MinElute Gel Extraction kit following the manufacturer's instructions. Briefly, Buffer QG was added to cover the agarose in the tube. The agarose was allowed to completely dissolve. The color of the Buffer QG was maintained by adjusting the pH according to the Qiagen instructions to minimize sample loss. The columns were eluded with 10 μl of Buffer EB which was pre-warmed at 55° C. The eluates were pooled to produce 20 μl of gDNA library.
8. One 1 μL aliquot of each isolated DNA library was analyzed using a BioAnalyzer DNA 1000 LabChip to assess the exact distribution of the DNA library population.

Step 5: Strand Displacement and Extension of Nicked Double Stranded DNA Library

These two “Gaps” or “nicks” can be filled in by using a strand displacing DNA polymerase.
1. In a 0.2 ml tube, 19 μl gel-extracted DNA library, 40 μl nH₂O, 8 μl 10× ThermoPol Reaction Buffer, 8 μl BSA (1 mg/ml), 2 μl dNTPs (10 mM), and 3 [μl Bst I Polymerase (8 U/μl) were added in order.
2. The samples were mixed well and placed in a thermal cycler and incubated using the Strand Displacement incubation program: “BST”. BST program for stand displacement and extension of nicked double-stranded DNA:
   (1) Incubate at 65° C., 30 minutes;
   (2) Incubate at 80° C., 10 minutes;
   (3) Incubate at 58° C., 10 minutes; and
   (4) Hold at 14° C.
3. One 1 μL aliquot of the Bst-treated DNA library was run using a BioAnalyzer DNA 1000 LabChip.

Step 6: Preparation of Streptavidin Beads
1. 100 μl Dynal M-270 Streptavidin beads were washed two times with 200 μl of 1× Binding Buffer (1 M NaCl, 0.5 mM EDTA, 5 mM Tris, pH 7.5) by applying the magnetic beads to the MPC.
2. The beads were resuspended in 100 μl 2× Binding buffer, then the remaining 79 μl of the Bst-treated DNA sample (from Step 5) and 20 μl water was added.
3. The bead solution was mixed well and placed on a tube rotator at RT for 20 minutes. The bead mixtures were washed, using the MPC, two times with 100 μl of 1× Binding Buffer, then washed two times with nH₂O. Binding & Washing (B&W) Buffer (2× and 1×): 2× B&W buffer was prepared by mixing 10 mM Tris.HCl (pH 7.5), 1 mM EDTA, and 2 M NaCl. The reagents were combined as listed above and mixed thoroughly. The solution can be stored at RT for 6 months; 1× B&W buffer was prepared by mixing 2× B&W buffer with nH₂O, 1:1. The final concentrations were half the above, i.e., 5 mM Tris.HCl (pH 7.5), 0.5 mM EDTA, and 1 M NaCl.

Step 7: Isolation of single-stranded DNA Library using Streptavidin Beads

Double-stranded genomic DNA fragment pools will have adaptors bound in the following possible configurations:
Universal Adaptor A—gDNA Fragment—Universal Adaptor A
Universal Adaptor B—gDNA Fragment—Universal Adaptor A*
Universal Adaptor A—gDNA Fragment—Universal Adaptor B*
Universal Adaptor B—gDNA Fragment—Universal Adaptor B

Because only the Universal Adaptor B has a 5′ biotin moiety, magnetic streptavidin-containing beads can be used to bind all gDNA library species that possess the Universal Adaptor B. To isolate the single-stranded population, the bead-bound double-stranded DNA is treated with a sodium hydroxide solution that serves to disrupt the hydrogen bonding between the complementary DNA strands.
1. 250 μl Melt Solution (0.125 M NaOH, 0.1 M NaCl)was added to washed beads from Step 6 above.
2. The bead solution was mixed well and the bead mixture was incubated at room temperature for 10 minutes on a tube rotator.
3. A Dynal MPC (magnetic particle concentrator) was used, the pellet beads were carefully removed, and the supernatant was set aside. The 250-μl supernatant included the single-stranded DNA library.
4. In a separate tube, 1250 μl PB (from QiaQuick Purification kit) was added and the solution was neutralized by adding 9 μl of 20% acetic acid.
5. Using a Dynal MPC, beads from the 250-μl supernatant including the single-stranded gDNA library were pelleted and the supernatant was carefully removed and transferred to the freshly prepared PB/acetic acid solution.
6. The 1500 μl solution was purified using a single QiaQuick purification spin column (load sample through same column two times at 750 μl per load). The single-stranded DNA library was eluted with 50 μl EB.

Step 8a: Single-stranded gDNA Quantitation using Pyrophosphate Sequencing.
1. In a 0.2 ml tube, the following reagents were added in order:
   25 μl single-stranded gDNA
   1 μl MMP2B sequencing primer
   14 μl Library Annealing Buffer
   40 μl total
2. The DNA was allowed to anneal using the ANNEAL-S Program (see Appendix, below).
3. The samples were run on PSQ (pyrophosphate sequencing jig) to determine the number of picomoles of template in each sample (see below). Methods of sequencing can be found in U.S. Pat. No. 6,274,320; U.S. Pat. No. 4,863,849; U.S. Pat. No. 6,210,891; and U.S. Pat. No. 6,258,568, the disclosures of which are incorporated in toto herein by reference. Calculations were performed to determine the number of single-stranded gDNA template molecules per microliter. The remaining 25 μL of prepared single-stranded gDNA library was used for amplification and subsequent sequencing (approximately 1×10⁶ reactions). Other methods of quantitating of DNA are known.

Step 9: Dilution and Storage of Single-Stranded gDNA library

The single-stranded gDNA library was eluted and quantitated in Buffer EB. To prevent degradation, the single-stranded gDNA library was stored frozen at −20° C. in the presence of EDTA. After quantitation, an equal volume of 10 mM TE was added to the library stock. All subsequent dilutions was in TE. The yield was as follows:
Remaining final volume of ssDNA library following PSQ analysis=25 μl.
Remaining final volume of ssDNA library following LabChip analysis=47 μl.
For the initial stock dilution, single-stranded gDNA library was diluted to 100 million molecules/μl in 133 Library-Grade Elution Buffer. Aliquots of single-stranded gDNA library were prepared for common use. For this, 200,000 molecules/μl were diluted in 1× Library-Grade Elution Buffer and 20 μl aliquots were measured. Single-use library aliquots were stored at −20° C.

Step 10: Emulsion Polymerase Chain Reaction

Bead emulsion PCR was performed as described in U.S. patent application Ser. No. 06/476,504 filed Jun. 6, 2003, incorporated herein by reference in its entirety.

Reagent Preparation

The Stop Solution (50 mM EDTA) included 100 μl of 0.5 M EDTA mixed with 900 μl of nH₂O to obtain 1.0 ml of 50 mM EDTA solution. For 10 mM dNTPs, (10 μl dCTP (100 mM), 10 μI dATP (100 mM), 10 μl dGTP (100 mM), and 10 μl dTTP (100 mM) were mixed with 60 μl molecular biology grade water. All four 100 mM nucleotide stocks were thawed on ice. Then, 10 μl of each nucleotide was combined with 60 μl of nH₂O to a final volume of 100 μl, and mixed thoroughly. Next, 1 ml aliquots were dispensed into 1.5 ml microcentrifuge tubes. The stock solutions could be stored at −20° C. for one year.

The 10× Annealing buffer included 200 mM Tris (pH 7.5) and 50 mM magnesium acetate. For this solution, 24.23 g Tris was added to 800 ml nH₂O and the mixture was adjusted to pH 7.5. To this solution, 10.72 g of magnesium acetate was added and dissolved completely. The solution was brought up to a final volume of 1000 ml and could be stored at 4° C. for 1 month. The 10×TE included 100 mM Tris.HCl (pH 7.5) and 50 mM EDTA. These reagents were added together and mixed thoroughly. The solution could be stored at room temperature for 6 months.

### Example 5

### Primer Design

As discussed above, the universal adaptors are designed to include: 1) a set of unique PCR priming regions that are typically 20 bp in length (located adjacent to (2)); 2) a set of unique sequencing priming regions that are typically 20 bp in length; and 3) optionally followed by a unique discriminating key sequence consisting of at least one of each of the four deoxyribonucleotides (i.e., A, C, G, T). The probability of cross-hybridization between primers and unintended regions of the genome of interest is increased as the genome size increases and length of a perfect match with the primer decreases. However, this potential interaction with a cross-hybridizing region (CHR) is not expected to produce problems for the reasons set forth below.

In a preferred embodiment of the present invention, the single-stranded DNA library is utilized for PCR amplification and subsequent sequencing. Sequencing methodology requires random digestion of a given genome into 150 to 500 base pair fragments, after which two unique bipartite primers (composed of both a PCR and sequencing region) are ligated onto the 5′ and 3′ ends of the fragments (FIG. 18). Unlike typical PCR amplifications where an existing section of the genome is chosen as a priming site based on melting temperature (Tₘ), uniqueness of the priming sequence within the genome and proximity to the particular region or gene of interest, the disclosed process utilizes synthetic priming sites that necessitates careful de novo primer design.

Tetramer Selection:

Strategies for de novo primer design are found in the published literature regarding work conducted on molecular tags for hybridization experiments (see, Hensel, M. and D. W. Holden, Molecular genetic approaches for the study of virulence in both pathogenic bacteria and fungi. Microbiology, 1996. 142(Pt 5): p. 1049-58; Shoemaker, D. D., et al., Quantitative phenotypic analysis of yeast deletion mutants using a highly parallel molecular bar-coding strategy. Nat Genet, 1996. 14(4): p. 450-6) and PCR/LDR (polymerase chain reaction/ligation detection reaction) hybridization primers (see, Gerry, N. P., et al., Universal DNA microarray method for multiplex detection of low abundance point mutations. Journal of Molecular Biology, 1999. 292: p. 251-262; Witowski, N. E., et al., Microarray-based detection of select cardiovascular disease markers. BioTechniques, 2000. 29(5): p. 936-944.).

The PCR/LDR work was particularly relevant and focused on designing oligonucleotide “zipcodes”, 24 base primers comprised of six specifically designed tetramers with a similar final Tₘ. (see, Gerry, N. P., et al., Universal DNA microarray method for multiplex detection of low abundance point mutations. Journal of Molecular Biology, 1999. 292: p. 251-262; U.S. Pat. No. 6,506,594). Tetrameric components were chosen based on the following criteria: each tetramer differed from the others by at least two bases, tetramers that induced self-pairing or hairpin formations were excluded, and palindromic (AGCT) or repetitive tetramers (TATA) were omitted as well. Thirty-six of the 256 (4⁴) possible permutations met the necessary requirements and were then subjected to further restrictions required for acceptable PCR primer design (Table 1).

TABLE 1

| | | | | | | | | | | | | | | | | |
|---|---|---|---|---|---|---|---|---|---|---|---|---|---|---|---|---|
| 6. | TA | TC | TG | TA | CT | CC | CG | CA | GT | GC | GG | GA | AT | AC | AG | AA |

| | | | | | | | | | | | | | | | | |
|---|---|---|---|---|---|---|---|---|---|---|---|---|---|---|---|---|
| TT | TTTT | TTTC | TTTG | TTTA | TTCT | TTCC | TTCG | TTCA | TTGT | TTGC | TTGG | TTGA | TTAT | TTAC | TTAG | TTAA |
| TC | TCTT | TCTC | TCTG | TCTA | TCCT | TCCC | TCCG | TCCA | TCGT | TCGC | TCGG | TCGA | TCAT | TCAC | TCAG | TCAA |
| TG | TGTT | TGTC | TGTG | TGTA | TGCT | TGCC | TGCG | TGCA | TGGT | TGGC | TGGG | TGGA | TGAT | TGAC | TGAG | TGAA |
| TA | TATT | TATC | TATG | TATA | TACT | TACC | TACG | TACA | TAGT | TAGC | TAGG | TAGA | TAAT | TAAC | TAAG | TAAA |
| CT | CTTT | CTTC | CTTG | CTTA | CTCT | CTCC | CTOG | CTCA | CTGT | CTGC | CTGG | CTGA | CTAT | CTAC | CTAG | CTAA |
| CC | CCTT | CCTC | CCTG | CCTA | CCCT | CCCC | CCCG | CCCA | CCGT | CCGC | CCGG | CCGA | CCAT | CCAC | CCAG | CCAA |
| CG | CGTT | CGTC | CGTG | CGTA | CGCT | CGCC | CGCG | CGCA | CGGT | CGGC | CGGG | CGGA | CGAT | CGAC | CGAG | CGAA |
| CA | CATT | CATC | CATG | CATA | CACT | CACC | CACG | CACA | CAGT | CAGC | CAGG | CAGA | CAAT | CAAC | CAAG | CAAA |
| GT | GTTT | GTTC | GTTG | GTTA | GTCT | GTCC | GTCG | GTCA | GTGT | GTGC | GTGG | GTGA | GTAT | GTAC | GTAG | GTAA |
| GC | GCTT | GCTC | GCTG | GCTA | GCCT | GCCC | GCCG | GCCA | GCGT | GCGC | GCGG | GCGA | GCAT | GCAC | GCAG | GCAA |
| GG | GGTT | GGTC | GGTG | GGTA | GGCT | GGCC | GGCG | GGCA | GGGT | GGGC | GGGG | GGGA | GGAT | GGAC | GGAG | GGAA |
| GA | GATT | GATC | GATG | GATA | GACT | GACC | GACG | GACA | GAGT | GAGC | GAGG | GAGA | GAAT | GAAC | GAAG | GAAA |
| AT | ATTT | ATTC | ATTG | ATTA | ATCT | ATCC | ATCG | ATCA | ATGT | ATGC | ATGG | ATGA | ATAT | ATAC | ATAG | ATAA |
| AC | ACTT | ACTC | ACTG | ACTA | ACCT | ACCC | ACCG | ACCA | ACGT | ACGC | ACGG | ACGA | ACAT | ACAC | ACAG | ACAA |
| AG | AGTT | AGTC | AGTG | AGTA | AGCT | AGOC | AGOG | AGCA | AGGT | AGGC | AGGG | AGGA | AGAT | AGAC | AGAG | AGAA |
| AA | AATT | AATC | AATG | AATA | AACT | AACC | AACG | AACA | AAGT | AAGC | AAGG | AAGA | AAAT | AAAC | AAAG | AAAA |

The table shows a matrix demonstrating tetrameric primer component selection based on criteria outlined by Gerry et al. 1999. J. Mol. Bio. 292: 251-262. Each tetramer was required to differ from all others by at least two bases. The tetramers could not be palindromic or complimentary with any other tetramer. Thirty-six tetramers were selected (bold, underlined); italicized sequences signal palindromic tetramers that were excluded from consideration.

Primer Design:

The PCR primers were designed to meet specifications common to general primer design (see, Rubin, E. and A. A. Levy, A mathematical model and a computerized simulation of PCR using complex template Nucleic Acids Res, 1996. 24(18): p. 3538-45; Buck, G. A., et al., Design strategies and performance of custom DNA sequencing primers. Biotechniques, 1999. 27(3): p. 528-36), and the actual selection was conducted by a computer program, MMP. Primers were limited to a length of 20 bases (5 tetramers) for efficient synthesis of the total bipartite PCR/sequencing primer. Each primer contained a two base GC clamp on the 5′ end, and a single GC clamp on the 3′ end (Table 2), and all primers shared similar Tₘ (±2° C.) (FIG. 19). No hairpinning within the primer (internal hairpin stem ΔG>−1.9 kcal/mol) was permitted. Dimerization was also controlled; a 3 base maximum acceptable dimer was allowed, but it could occur in final six 3′ bases, and the maximum allowable ΔG for a 3′ dimer was −2.0 kcal/mol. Additionally, a penalty was applied to primers in which the 3′ ends were too similar to others in the group, thus preventing cross-hybridization between one primer and the reverse complement of another.

TABLE 2

| | | | | | |
|---|---|---|---|---|---|
| 7. | 1-pos | 2-pos | 3-pos | 4-pos | 5-pos |

| | | | | | |
|---|---|---|---|---|---|
| 1 | CCAT | TGAT | TGAT | TGAT | ATAC |
| 2 | CCTA | CTCA | CTCA | CTCA | AAAG |
| 3 | CGAA | TACA | TACA | TACA | TTAG |
| 4 | CGTT | AGCC | AGCC | AGCC | AATC |
| 5 | GCAA | GACC | GACC | GACC | TGTC |
| 6 | GCTT | TCCC | TCCC | TCCC | AGTG |
| 7 | GGAC | ATCG | ATCG | ATCG | CTTG |
| 8 | GGTA | CACG | CACG | CACG | GATG |
| 9 | | TGCG | TGCG | TGCG | TCTG |
| 10 | | ACCT | ACCT | ACCT | |
| 11 | | GTCT | GTCT | GTCT | |
| 12 | | AGGA | AGGA | AGGA | |
| 13 | | TTGA | TTGA | TTGA | |
| 14 | | CAGC | CAGC | CAGC | |
| 15 | | GTGC | GTGC | GTGC | |
| 16 | | ACGG | ACGG | ACGG | |
| 17 | | CTGT | CTGT | CTGT | |
| 18 | | GAGT | GAGT | GAGT | |
| 19 | | TCGT | TCGT | TCGT | |

Table 2 shows possibly permutations of the 36 selected tetrads providing two 5′ and a single 3′ C/C clamp. The internal positions are composed of remaining tetrads. This results in 8 ×19×19×19×9 permutations, or 493,848 possible combinations. FIG. 19 shows first pass, Tₘ based selection of acceptable primers, reducing field of 493,848 primers to 56,246 candidates with Tₘ of 64 to 66° C.

8.

TABLE 3
The probability of perfect sequence matches for primers increases
with decreasing match length requirements an size of the genome of

| | Perfect | % chance | | % |
|---|---|---|---|---|
| | match | for match | % chance for match in | chance for |
| | probability | in Adeno | % bacterial database ˜ | match in |
| Match | (1/(4\hat{ }length) | 35K | bases | ˜3B |
| 20 | 9.1E−13 | 0.00% | 0.04% | 0.27% |
| 19 | 7.3E−12 | 0.00% | 0.65% | 4.32% |
| 18 | 4.4E−11 | 0.00% | 5.76% | 34.37% |
| 17 | 2.3E−10 | 0.00% | 35.69% | 99.17% |
| 16 | 1.2E−09 | 0.02% | 97.52% | >100% |
| 15 | 5.6E−09 | 0.12% | >100% | >100% |
| 14 | 2.6E−08 | 0.64% | >100% | >100% |
| 13 | 1.2E−07 | 3.29% | >100% | >100% |
| 12 | 5.4E−07 | 15.68% | >100% | >100% |
| 11 | 2.4E−06 | 58.16% | >100% | >100% |
| 10 | 1.0E−05 | 99.35% | >100% | >100% |
| 9 | 4.6E−05 | 99.77% | >100% | >100% |
| 8 | 2.0E−04 | >100% | >100% | >100% |
| 7 | 8.5E−04 | >100% | >100% | >100% |
| 6 | 3.7E−03 | >100% | >100% | >100% |
| 5 | 1.6E−02 | >100% | >100% | >100% |
| 4 | 6.4E−02 | >100% | >100% | >100% |
| 3 | 2.5E−01 | >100% | >100% | >100% |
| 2 | 7.1E−01 | >100% | >100% | >100% |
| 1 | 1.0E+00 | >100% | >100% | >100% |

### Example 3

### DNA Sample Preparation For Sequence-Based Karyotyping

Preparation of DNA by Nebulization

The purpose of the Nebulization step is to fragment a large stretch of DNA such as a whole genome or a large portion of a genome into smaller molecular species that are amenable to DNA sequencing. This population of smaller-sized DNA species generated from a single DNA template is referred to as a library. Nebulization shears double-stranded template DNA into fragments ranging from 50 to 900 base pairs. The sheared library contains single-stranded ends that are end-repaired by a combination of T4 DNA polymerase, E. coli DNA polymerase I (Klenow fragment), and T4 polynucleotide kinase. Both T4 and Klenow DNA polymerases are used to “fill-in” 3′ recessed ends (5′ overhangs) of DNA via their 5′-3′ polymerase activity. The single-stranded 3′-5′ exonuclease activity of T4 and Klenow polymerases will remove 3′ overhang ends and the kinase activity of T4 polynucleotide kinase will add phosphates to 5′ hydroxyl termini.

The sample was prepared as follows:
1. 15 μg of gDNA (genomic DNA) was obtained and adjusted to a final volume of 100 μl in 10 mM TE (10 mM Tris, 0.1 mM EDTA, pH 7.6; see reagent list at the end of section). The DNA was analyzed for contamination by measuring the O.D. _{260/280} ratio, which was 1.8 or higher. The final gDNA concentration was expected to be approximately 300 μg/ml.
2. 1600 μl of ice-cold Nebulization Buffer (see end of section) was added to the gDNA.
3. The reaction mixture was placed in an ice-cold nebulizer (CIS-US, Bedford, Mass.).
4. The cap from a 15 ml snap cap falcon tube was placed over the top of the nebulizer (FIG. 51028A).
5. The cap was secured with a clean Nebulizer Clamp assembly, consisting of the fitted cover (for the falcon tube lid) and two rubber O-rings (FIG. 20).
6. The bottom of the nebulizer was attached to a nitrogen supply and the entire device was wrapped in parafilm (FIG. 20).
7. While maintaining nebulizer upright (as shown in FIG. 20), 50 psi (pounds per square inch) of nitrogen was applied for 5 minutes. The bottom of the nebulizer was tapped on a hard surface every few seconds to force condensed liquid to the bottom.
8. Nitrogen was turned off after 5 minutes. After the pressure had normalized (30 seconds), the nitrogen source was remove from the nebulizer.
9. The parafilm was removed and the nebulizer top was unscrewed. The sample was removed and transferred to a 1.5 ml microcentrifuge tube.
10. The nebulizer top was reinstalled and the nebulizer was centrifuged at 500 rpm for 5 minutes.
11. The remainder of the sample in the nebulizer was collected. Total recovery was about 700 μl.
12. The recovered sample was purified using a QIAquick column (Qiagen Inc., Valencia, Calif.) according to manufacturer's directions. The large volume required the column to be loaded several times. The sample was eluted with 30 μl of Buffer EB (10 mM Tris HCl, pH 8.5;supplied in Qiagen kit) which was pre-warmed at 55° C.
13. The sample was quantitated by UV spectroscopy (2 μl in 198 μl water for 1:100 dilution).

Enzymatic Polishing

Nebulization of DNA templates yields many fragments of DNA with frayed ends. These ends are made blunt and ready for ligation to adaptor fragments by using three enzymes, T4 DNA polymerase, E. coli DNA polymerase (Klenow fragment) and T4 polynucleotide kinase.

The sample was prepared as follows:
1. In a 0.2 ml tube the following reagents were added in order:
   28 μl purified, nebulized gDNA fragments
   5 μl water
   5 μl 10×T4 DNA polymerase buffer
   5 μl BSA (1 mg/ml)
   2 μl dNTPs (10 mM)
   5 μl T4 DNA polymerase (3 units/μl)
   50 μl final volume
2. The solution of step 1 was mixed well and incubated at 25° C. for 10 minutes in a MJ thermocycler (any accurate incubator may be used).
3. 1.25 μl E. coli DNA polymerase (Klenow fragment) (5 units/ml) was added.
4. The reaction was mixed well and incubated in the MJ thermocycler for 10 minutes at 25° C. and for an additional 2 hrs at 16° C.
5. The treated DNA was purified using a QiaQuick column and eluted with 30 μl of Buffer EB (10 mM Tris HCl, pH 8.5) which was pre-warmed at 55° C.
6. The following reagents were combined in a 0.2 ml tube:
   30 μl Qiagen purified, polished, nebulized gDNA fragments
   5 μl water
   5 μl 10×T4 PNK buffer
   5 μl ATP (10 mM)
   5 μl T4 PNK (10 units/ml)
   50 μl final volume
7. The solution was mixed and placed in a MJ thermal cycler using the T4 PNK program for incubation at 37° C. for 30 minutes, 65° C. for 20 minutes, followed by storage at 14° C.
8. The sample was purified using a QiaQuick column and eluted in 30 μl of Buffer EB which was pre-warmed at 55° C.
9. A 2 μl aliquot of the final polishing reaction was held for analysis using a BioAnalyzer DNA 1000 LabChip (see below).

Ligation of Adaptors

The procedure for ligating the adaptors was performed as follows:
1. In a 0.2 ml tube the following reagents were added in order:
   20.6 μl molecular biology grade water
   28 μl digested, polished gDNA Library
   60 μl 2× Quick Ligase Reaction Buffer
   1.8 μl MMP (200 pmol/μl) Universal Adaptor set
   9.6 μl Quick Ligase
   120 μl total

The above reaction was designed for 5 μg and was scaled depending on the amount of gDNA used.
2. The reagents were mixed well and incubated at 25° C. for 20 minutes. The tube was on ice until the gel was prepared for agarose gel electrophoresis.

Gel Electrophoresis and Extraction of Adapted gDNA Library

The procedure described below was used to isolated fragments of 250 bp to 500 bp.

A 150 ml agarose gel was prepared to include 2% agarose, 1× TBE, and 4.5 μl ethidium bromide (10 mg/ml stock). The ligated DNA was mixed with 10× Ready Load Dye and loaded onto the gel. In addition, 10 μl of a 100-bp ladder (0.1 μg/μl) was loaded on two lanes away from the ligation reaction flanking the sample. The gel was electrophoresed at 100 V for 3 hours. When the gel run was complete, the gel was removed from the gel box, transferred to a GelDoc, and covered with plastic wrap. The DNA bands were visualized using the Prep UV light. A sterile, single-use scalpel, was used to cut out a library population from the agarose gel with fragment sizes of 250-500 bp. This process was done as quickly as possible to prevent nicking of DNA. The gel slices were placed in a 15 ml falcon tube. The agarose-embedded gDNA library was isolated using a Qiagen MinElute Gel Extraction kit. Aliquots of each isolated gDNA library were analyzed using a BioAnalyzer DNA 1000 LabChip to assess the exact distribution of the gDNA library population.

Strand Displacement and Extension of the gDNA Library and Isolation of the Single Stranded gDNA Library Using Streptavidin Beads

Strand displacement and extension of nicked double-stranded gDNA library was performed as described in Example 1, with the exception that the Bst-treated samples were incubated in the thermal cycler at 65° C. for 30 minutes and placed on ice until needed. Streptavidin beads were prepared as described in Example 1, except that the final wash was performed using two washes with 200 μl 1× Binding buffer and two washes with 200 μl nH₂O. Single-stranded gDNA library was isolated using streptavidin beads as follows. Water from the washed beads was removed and 250 [μl of Melt Solution (see below) was added. The bead suspension was mixed well and incubated at room temperature for 10 minutes on a tube rotator. In a separate tube, 1250 μl of PB (from the QiaQuick Purification kit) and 9 μl of 20% acetic acid were mixed. The beads in 250 μl Melt Solution were pelleted using a Dynal MPC and the supernatant was carefully removed and transferred to the freshly prepared PB/acetic acid solution. DNA from the 1500 μl solution was purified using a single MinElute purification spin column. This was performed by loading the sample through the same column twice at 750 μl per load. The single stranded gDNA library was eluted with 15 μl of Buffer EB which was pre-warmed at 55° C.

Single Strand gDNA Quantitation and Storage

Single-stranded gDNA was quantitated using RNA Pico 6000 LabChip according to manufacturer's instructions.

Dilution and storage of the single stranded gDNA library was performed as described in Example 1. The yield was as follows:
Remaining final volume of ssDNA library following LabChip analysis=12 μl.
Remaining final volume of ssDNA library following RiboGreen analysis=9 μl.
Final volume of ssDNA library after the addition of TE=18 μl.

An equal volume of TE was added to single-stranded gDNA library stock. Single-stranded gDNA library to 1×10⁸ molecules/μl in Buffer TE. Stock was diluted (1/500) to 200,000 molecules/μl in TE and 20 μl aliquots were prepared.

Library Fragment Size Distribution After Nebulization

Typical results from Agilent 2100 DNA 1000 LabChip analysis of 1 μl of the material following Nebulization and polishing are around 50 to 900 base pairs.

Reagents

Unless otherwise specified, the reagents listed in the Examples represent standard reagents that are commercially available. For example, Klenow, T4 DNA polymerase, T4 DNA polymerase buffer, T4 PNK, T4 PNK buffer, Quick T4 DNA Ligase, Quick Ligation Buffer, Bst DNA polymerase (Large Fragment) and ThermoPol reaction buffer are available from New England Biolabs (Beverly, Mass.). dNTP mix is available from Pierce (Rockford, Ill.). Agarose, UltraPure TBE, BlueJuice gel loading buffer and Ready-Load 100 bp DNA ladder may be purchased from Invitrogen (Carlsbad, Calif.). Ethidium Bromide and 2-Propanol may be purchased from Fisher (Hampton, N.H.). RNA Ladder may be purchased from Ambion (Austin, Tex.). Other reagents are either commonly known and/or are listed below:

Melt Solution:

| Irgredient | Quantity Required | | Vendor | Stock Number |
|---|---|---|---|---|
| NaCl (5 M) | 200 | μl | Invitrogen | 24740-011 |
| NaOH (10 N) | 125 | μl | Fisher | SS255-1 |
| molecular biology | 9.675 | ml | Eppendorf | 0032-006-205 |
| grade water | | | | |

The Melt Solution included 100 mM NaCl, and 125 mM NaOH. The listed reagents were combined and mixed thoroughly. The solution could be stored at RT for six months.

Binding & Washing (B&W) Buffer (2× and 1×):

| Ingredient | Quantity Required | | Vendor | Stock Number |
|---|---|---|---|---|
| UltraPure Tris-HCl | 250 | μl | Invitrogen | 15567-027 |
| (pH 7.5, 1 M) | | | | |
| EDTA (0.5 M) | 50 | μl | Invitrogen | 15575-020 |
| NaCl (5 M) | 10 | ml | Invitrogen | 24740-011 |
| molecular biology | 14.7 | ml | Eppendorf | 0032-006-205 |
| grade water | | | | |

The 2× B&W buffer included final concentrations of 10 mM Tris-HCl (pH 7.5), 1 mM EDTA, and 2 M NaCl. The listed reagents were combined by combined and mixed thoroughly. The solution could be stored at RT for 6 months. The 1× B&W buffer was prepared by mixing 2× B&W buffer with picopure H₂O, 1:1. The final concentrations was half of that listed the above, i.e., 5 mM Tris-HCl (pH 7.5), 0.5 mM EDTA, and 1 M NaCl.

Other buffers included the following. 1× T4 DNA Polymerase Buffer: 50 mM NaCl, 10 mM Tris-HCl, 10 mM MgCl2, 1 mM dithiothreitol (pH 7.9 @ 25° C.). TE: 10 mM Tris, 1 mM EDTA.

Special Reagent Preparation:

TE (10 mM):

| Ingredient | Quantity Required | Vendor | Stock Number |
|---|---|---|---|
| TE (1 M) | 1 ml | Fisher | BP1338-1 |
| molecular biology | 99 ml | Eppendorf | 0032-006-205 |
| grade water | | | |

Nebulization Buffer:

| Ingredient | Quantity Required | Vendor | Stock Number |
|---|---|---|---|
| Glycerol | 53.1 ml | Sigma | G5516 |
| molecular biology | 42.1 ml | Eppendorf | 0032-006-205 |
| grade water | | | |
| UltraPure Tris-HCl | 3.7 ml | Invitrogen | 15567-027 |
| (pH 7.5, 1M) | | | |
| EDTA (0.5M) | 1.1 ml | Sigma | M-10228 |

ATP (10 mM):

| Ingredient | Quantity Required | Vendor | Stock Number |
|---|---|---|---|
| ATP (100 mM) | 10 μl | Roche | 1140965 |
| molecular biology | 90 μl | Eppendorf | 0032-006-205 |
| grade water | | | |

BSA (1 mg/ml):

| Ingredient | Quantity Required | Vendor | Stock Number |
|---|---|---|---|
| BSA (10 mg/ml) | 10 μl | NEB | M0203 kit |
| Molecular Biology | 90 μl | Eppendorf | 0032-006-205 |
| Grade water | | | |

Library Annealing Buffer, 10×:

| Ingredient | Quantity Req. | | Vendor | Stock No. |
|---|---|---|---|---|
| UltraPure Tris-HCl | 200 | ml | Invitrogen | 15567-027 |
| (pH 7.5, 1 M) | | | | |
| Magnesium acetate, | 10.72 | g | Fisher | BP-215-500 |
| enzyme grade (1 M) | | | | |
| Molecular Biology | ˜1 | L | Eppendorf | 0032-006-205 |
| Grade water | | | | |

The 10× Annealing Buffer included 200 mM Tris (pH 7.5) and 50 mM magnesium acetate. For this buffer, 200 ml of Tris was added to 500 ml picopure H₂O. Next, 10.72 g of magnesium acetate was added to the solution and dissolved completely. The solution was adjusted to a final volume of 1000 ml.

Adaptors:

Adaptor “A” (400 μM):

| | Quantity | | |
|---|---|---|---|
| Ingredient | Req. | Vendor | Stock No. |
| Adaptor A (sense; | 10.0 μl | IDT | custom |
| HPLC-purified, | | | |
| phosphorothioate linkages, | | | |
| 44 bp, 1000 pmol/μl) | | | |
| Adaptor A (antisense; | 10.0 μl | IDT | custom |
| HPLC-purified, | | | |
| Phosphorothioate linkages, | | | |
| 40 bp, 1000 pmol/μl) | | | |
| Annealing buffer (10×) | 2.5 μl | 454 Corp. | previous |
| | | | table |
| molecular biology grade water | 2.5 μl | Eppendorf | 0032-006-205 |

For this solution, 10 μl of 1000 pmol/μl Adaptor A (44 bp, sense) was mixed with 10 μl of 1000 pmol/μl Adaptor A (40 bp, antisense), 2.5 μl of 10× Library Annealing Buffer, and 2.5 μl of water (V_{f}=25 μl). The adaptors were annealed using the ANNEAL-A program (see Appendix, below) on the Sample Prep Lab thermal cycler. More details on adaptor design are provided in the Appendix.

Adaptor “B” (400 μM):

| | Quantity | | |
|---|---|---|---|
| Ingredient | Req. | Vendor | Stock No. |
| Adaptor B (sense; | 10 μl | IDT | Custom |
| HPLC-purified, | | | |
| phosphorothioate | | | |
| linkages, 40 bp, | | | |
| 1000 pmol/μl)) | | | |
| Adaptor B (anti; HPLC-purified, | 10 μl | IDT | Custom |
| phosphorothioate linkages, | | | |
| 5′Biotinylated, 44 bp, | | | |
| 1000 pmol/μl) | | | |
| Annealing buffer (10X) | 2.5 μl | 454 Corp. | previous table |
| molecular biology grade water | 2.5 μl | Eppendorf | 0032-006-205 |

For this solution, 10 μl of 1000 pmol/μl Adaptor B (40 bp, sense) was mixed with 10 μl of 1000 pmol/μl Adaptor B (44 bp, anti), 2.5 μl of 10× Library Annealing Buffer, and 2.5 μl of water (V_{f}=25 μl). The adaptors were annealed using the ANNEAL-A program (see Appendix) on the Sample Prep Lab thermal cycler. After annealing, adaptor “A” and adaptor “B” (V_{f}=50 μl) were combined. Adaptor sets could be stored at −20° C. until use.

20% Acetic Acid:

| | Quantity | | |
|---|---|---|---|
| Ingredient | Required | Vendor | Stock Number |
| acetic acid, glacial | 2 ml | Fisher | A35-500 |
| molecular biology grade water | 8 ml | Eppendorf | 0032-006-205 |

Adaptor Annealing Program:
ANNEAL-A program for primer annealing:
(1) Incubate at 95° C., 1 min;
(2) Reduce temperature to 15° C. at 0.1° C./sec; and
(3) Hold at 14° C.

T4 Polymerase/Klenow POLISH program for end repair:
(1) Incubate at 25° C., 10 minutes;
(2) Incubate at 16° C., 2 hours; and
(3) Hold at 4° C.

T4 PNK Program for end repair:
(1) Incubate at 37° C., 30 minutes;
(2) Incubate at 65° C., 20 minutes; and
(3) Hold at 14° C.

BST program for stand displacement and extension of nicked double-stranded gDNA:
(1) Incubate at 65° C., 30 minutes; and
(2) Hold at 14° C.

Step 9: Dilution and Storage of Single-Stranded DNA library

Single-stranded DNA library in EB buffer: remaining final volume=25 μl.

Initial Stock dilution was made as follows. Using Pyrosequencing (Pyrosequencing AB, Uppsala, Sweden) results, single-stranded DNA library was diluted to 100M molecules/EL in 1× Annealing Buffer (usually this was a 1:50 dilution).

Aliquots of single-stranded DNA Library were made for common use by diluting 200,000 molecules/EL in 1× Annealing Buffer and preparing 30 μL aliquots. Store at −20° C. Samples were utilized in emulsion PCR.

### 9. Reagent Preparation

Stop Solution (50 mM EDTA): 100 μl of 0.5 M EDTA was mixed with 900 μl of nH₂O to make 1.0 ml of 50 mM EDTA solution.

Solution of 10 mM dNTPs included 10 μl dCTP (100 mM), 10 μl dATP (100 mM), 10 μl dGTP (100 mM), and 10 μl dTTP (100 mM), 60 μl Molecular Biology Grade water, (nH₂O). All four 100 mM nucleotide stocks were thawed on ice. 10 μl of each nucleotide was combined with 60 μl of nH₂O to a final volume of 100 μl, and mixed thoroughly. 1 ml aliquots were dispensed into 1.5 ml microcentrifuge tubes, and stored at −20° C., no longer than one year.

Annealing buffer, 10×: 10× Annealing buffer included 200 mM Tris (pH 7.5) and 50 mM magnesium acetate. For this solution, 24.23 g Tris was added to 800 ml nH2O and adjusted to pH 7.5. To this, 10.72 g magnesium acetate was added and dissolved completely. The solution was brought up to a final volume of 1000 ml. The solution was able be stored at 4° C. for 1 month.

10× TE: 10× TE included 100 mM Tris.HCl (pH 7.5), and 50 mM EDTA. These reagents were added together and mixed thoroughly. The solution could be stored at room temperature for 6 months.

PCR Reaction Mix:

For 200 μl PCR reaction mixture (enough for amplifying 600,000 beads), the following reagents were combined in a 0.2 ml PCR tube:

10.

TABLE 4

| | Stock | Final | Microliters |
|---|---|---|---|
| HIFI Buffer | 10 X | 1 X | 20 |
| treated nucleotides | 10 mM | 1 mM | 20 |
| Mg | 50 mM | 2 mM | 8 |
| BSA | 10% | 0.1% | 2 |
| Tween 80 | 1% | 0.01% | 2 |
| Ppase | 2 U | 0.003 U | 0.333333 |
| Primer MMP1a | 100 μM | 0.625 μM | 1.25 |
| Primer MMP1b | 10 μM | 0.078 μM | 1.56 |
| Taq polymerase | 5 U | 0.2 U | 8 |
| Water | | | 136.6 |
| Total | | | 200 |

The tube was vortexed thoroughly and stored on ice until the beads are annealed with template.

DNA Capture Beads:
1. 600,000 DNA capture beads were transferred from the stock tube to a 1.5 ml microfuge tube. The exact amount used will depend on bead concentration of formalized reagent.
2. The beads were pelleted in a benchtop mini centrifuge and supernatant was removed.
3. Steps 4-11 were performed in a PCR Clean Room.
4. The beads were washed with 1 mL of 1× Annealing Buffer.
5. The capture beads were pelleted in the microcentrifuge. The tube was turned 180° and spun again.
6. All but approximately 10 μl of the supernatant was removed from the tube containing the beads. The beads were not disturbed.
7. 1 mL of 1× Annealing Buffer was added and this mixture was incubated for 1 minute. The beads were then pelleted as in step 5.
8. All but approximately 100 μL of the material from the tube was removed.
9. The remaining beads and solution were transferred to a PCR tube.
10. The 1.5 mL tube was washed with 150 μL of 1× Annealing Buffer by pipetting up and down several times. This was added to the PCR tube containing the beads.
11. The beads were pelleted as in step 5 and all but 10 μL of supernatant was removed, taking care to not disturb the bead pellet.
12. An aliquot of quantitated single-stranded template DNA (sstDNA) was removed. The final concentration was 200,000-sst DNA molecules/μl.
13. 3 μl of the diluted sstDNA was added to PCR tube containing the beads. This was equivalent to 600,000 copies of sstDNA.
14. The tube was vortexed gently to mix contents.
15. The sstDNA was annealed to the capture beads in a PCR thermocycler with the program 80Anneal stored in the EPCR folder on the MJ Thermocycler, using the following protocol:
16. 5 minutes at 65° C.;
17. Decrease by 0.1° C. /sec to 60° C.;
18. Hold at 60° C. for 1 minute;
19. Decrease by 0.1° C./sec to 50° C.;
20. Hold at 50° C. for 1 minute;
21. Decrease by 0.1° C./sec to 40° C.;
22. Hold at 40° C. for 1 minute;
23. Decrease by 0.1° C. /sec to 20° C.; and
24. Hold at 10° C. until ready for next step.
25. In most cases, beads were used for amplification immediately after template binding. If beads were not used immediately, they should were stored in the template solution at 4° C. until needed. After storage, the beads were treated as follows.
26. As in step 6, the beads were removed from the thermocycler, centrifuged, and annealing buffer was removed without disturbing the beads.
27. The beads were stored in an ice bucket until emulsification (Example 2).
28. The capture beads included, on average, 0.5 to 1 copies of sstDNA bound to each bead, and were ready for emulsification.

### Example 5

### Emulsification

A PCR solution suitable for use in this step is described below. For 200 μl PCR reaction mix (enough for amplifying 600K beads), the following were added to a 0.2 ml PCR tube:

| | Stock | Final | Microliters |
|---|---|---|---|
| HIFI Buffer | 10 X | 1 X | 20 |
| treated Nukes | 10 mM | 1 mM | 20 |
| Mg | 50 mM | 2 mM | 8 |
| BSA | 10% | 0.1% | 2 |
| Tween 80 | 1% | 0.01% | 2 |
| Ppase | 2 U | 0.003 U | 0.333333 |
| Primer MMP1a | 100 μM | 0.625 μM | 1.25 |
| Primer MMP1b | 10 μM | 0.078 μM | 1.56 |
| Tag | 5 U | 0.2 U | 8 |
| Water | | | 136.6 |
| Total | | | 200 |

This example describes how to create a heat-stable water-in-oil emulsion containing about 3,000 PCR microreactors per microliter. Outlined below is a protocol for preparing the emulsion.
1. 200 μl of PCR solution was added to the 600,000 beads (both components from Example 1).
2. The solution was pipetted up and down several times to resuspend the beads.
3. The PCR-bead mixture was allowed to incubate at room temperature for 2 minutes to equilibrate the beads with PCR solution.
4. 400 μl of Emulsion Oil was added to a UV-irradiated 2 ml microfuge tube.
5. An “amplicon-free” ¼″ stir magnetic stir bar was added to the tube of Emulsion Oil.
6. An amplicon-free stir bar was prepared as follows. A large stir bar was used to hold a ¼″ stir bar. The stir bar was then:
   Washed with DNA-Off (drip or spray);
   Rinsed with picopure water;
   Dried with a Kimwipe edge; and
   UV irradiated for 5 minutes.
7. The magnetic insert of a Dynal MPC-S tube holder was removed. The tube of Emulsion Oil was placed in the tube holder. The tube was set in the center of a stir plate set at 600 rpm.
8. The tube was vortexed extensively to resuspend the beads. This ensured that there was minimal clumping of beads.
9. Using a P-200 pipette, the PCR-bead mixture was added drop-wise to the spinning oil at a rate of about one drop every 2 seconds, allowing each drop to sink to the level of the magnetic stir bar and become emulsified before adding the next drop. The solution turned into a homogeneous milky white liquid with a viscosity similar to mayonnaise.
10. Once the entire PCR-bead mixture was been added, the microfuge tube was flicked a few times to mix any oil at the surface with the milky emulsion.
11. Stirring was continued for another 5 minutes.
12. Steps 9 and 10 were repeated.
13. The stir bar was removed from the emulsified material by dragging it out of the tube with a larger stir bar.
14. 10 μL of the emulsion was removed and placed on a microscope slide. The emulsion was covered with a cover slip and the emulsion was inspected at 50× magnification (10× ocular and 5× objective lens). A “good” emulsion was expected to include primarily single beads in isolated droplets (microreactors) of PCR solution in oil.
15. A suitable emulsion oil mixture with emulsion stabilizers was made as follows. The components for the emulsion mixture are shown in Table 5.

11.

TABLE 5

| | Quantity | | |
|---|---|---|---|
| Ingredient | Required | Source | Ref. Number |
| Sigma Light Mineral Oil | 94.5 g | Sigma | M-5904 |
| Atlox 4912 | 1 g | Uniqema | NA |
| Span 80 | 4.5 g | Uniqema | NA |

The emulsion oil mixture was made by prewarming the Atlox 4912 to 60° C. in a water bath. Then, 4.5 grams of Span 80 was added to 94.5 grams of mineral oil to form a mixture. Then, one gram of the prewarmed Atlox 4912 was added to the mixture. The solutions were placed in a closed container and mixed by shaking and inversion. Any sign that the Atlox was settling or solidifying was remedied by warming the mixture to 60° C., followed by additional shaking.

### Example

### Amplification

PCR was performed as follows:
The emulsion was transferred in 50-100 μL amounts into approximately 10 separate PCR tubes or a 96-well plate using a single pipette tip. For this step, the water-in-oil emulsion was highly viscous.
The plate was sealed, or the PCR tube lids were closed, and the containers were placed into in a MJ thermocycler with or without a 96-well plate adaptor.
The PCR thermocycler was programmed to run the following program:
   1 cycle (4 minutes at 94° C.)—Hotstart Initiation;
   40 cycles (30 seconds at 94° C., 30 seconds at 58° C., 90 seconds at 68° C.);
   25 cycles (30 seconds at 94° C., 6 minutes at 58° C.); and
   Storage at 14° C.

After completion of the PCR reaction, the amplified material was removed in order to proceed with breaking the emulsion and bead recovery.

### Example 7

### Breaking the Emulsion and Bead Recovery

1. All PCR reactions from the original 600 μl sample were combined into a single 1.5 ml microfuge tube using a single pipette tip. As indicated above, the emulsion was quite viscous. In some cases, pipetting was repeated several times for each tube. As much material as possible was transferred to the 1.5 ml tube.

2. The remaining emulsified material was recovered from each PCR tube by adding 50 μl of Sigma Mineral Oil into each sample. Using a single pipette tip, each tube was pipetted up and down a few times to resuspend the remaining material.

3. This material was added to the 1.5 ml tube containing the bulk of the emulsified material.

4. The sample was vortexed for 30 seconds.

5. The sample was spun for 20 minutes in the tabletop microfuge tube at 13.2K rpm in the Eppendorf microcentrifuge.

6. The emulsion separated into two phases with a large white interface. As much of the top, clear oil phase as possible was removed. The cloudy material was left in the tube. Often a white layer separated the oil and aqueous layers. Beads were often observed pelleted at the bottom of the tube.

7. The aqueous layer above the beads was removed and saved for analysis (gel analysis, Agilent 2100, and Taqman). If an interface of white material persisted above the aqueous layer, 20 microliters of the underlying aqueous layer was removed. This was performed by penetrating the interface material with a pipette tip and withdrawing the solution from underneath.

8. In the PTP Fabrication and Surface Chemistry Room Fume Hood, 1 ml of Hexanes was added to the remainder of the emulsion.

9. The sample was vortexed for 1 minute and spun at full speed for 1 minute.

10. In the PTP Fabrication and Surface Chemistry Room Fume Hood, the top, oil/hexane phase was removed and placed into the organic waste container.

11. 1 ml of 1× Annealing Buffer was added in 80% Ethanol to the remaining aqueous phase, interface, and beads.

12. The sample was vortexed for 1 minute or until the white substance dissolved.

13. The sample was centrifuged for 1 minute at high speed. The tube was rotated 180 degrees, and spun again for 1 minute. The supernatant was removed without disturbing the bead pellet.

14. The beads were washed with 1 ml of 1× Annealing Buffer containing 0.1% Tween 20 and this step was repeated.

### Example 8

### Single Strand Removal and Primer Annealing

1. The beads were washed with 1 ml of water, and spun twice for 1 minute. The tube was rotated 180° between spins. After spinning, the aqueous phase was removed.

2. The beads were washed with 1 ml of 1 mM EDTA. The tube was spun as in step 1 and the aqueous phase was removed.

3. 1 ml of 0.125 M NaOH was added and the sample was incubated for 8 minutes.

4. The sample was vortexed briefly and placed in a microcentrifuge.

5. After 6 minutes, the beads were pelleted as in step 1 and as much solution as possible was removed.

6. At the completion of the 8 minute NaOH incubation, 1 ml of 1× Annealing Buffer was added.

7. The sample was briefly vortexed, and the beads were pelleted as in step 1. As much supernatant as possible was removed, and another 1 ml of 1× Annealing buffer was added.

8. The sample was briefly vortexed, the beads were pelleted as in step 1, and 800 μl of 1× Annealing Buffer was removed.

9. The beads were transferred to a 0.2 ml PCR tube.

10. The beads were transferred and as much Annealing Buffer as possible was removed, without disturbing the beads.

11. 100 μl of 1× Annealing Buffer was added.

12. 4 μl of 100 μM sequencing primer was added. The sample was vortexed just prior to annealing.

13. Annealing was performed in a MJ thermocycler using the “80Anneal” program.

14. The beads were washed three times with 200 μl of 1× Annealing Buffer and resuspended with 100 μl of 1× Annealing Buffer.

15. The beads were counted in a Hausser Hemacytometer. Typically, 300,000 to 500,000 beads were recovered (3,000-5,000 beads/μL).

16. Beads were stored at 4° C. and could be used for sequencing for 1 week.

### Example 9

### Optional Enrichment Step

The beads may be enriched for amplicon containing bead using the following procedure. Enrichment is not necessary but it could be used to make subsequent molecular biology techniques, such as DNA sequencing, more efficient.

Fifty microliters of 10 μM (total 500 pmoles) of biotin-sequencing primer was added to the Sepharose beads containing amplicons from Example 5. The beads were placed in a thermocycler. The primer was annealed to the DNA on the bead by the thermocycler annealing program of Example 2.

After annealing, the sepharose beads were washed three times with Annealing Buffer containing 0.1% Tween 20. The beads, now containing ssDNA fragments annealed with biotin-sequencing primers, were concentrated by centrifugation and resuspended in 200 μl of BST binding buffer. Ten microliters of 50,000 unit/ml Bst-polymerase was added to the resuspended beads and the vessel holding the beads was placed on a rotator for five minutes. Two microliters of 10 mM dNTP mixture (i.e., 2.5 μl each of 10 mM dATP, dGTP, dCTP and dTTP) was added and the mixture was incubated for an additional 10 minutes at room temperature. The beads were washed three times with annealing buffer containing 0.1% Tween 20 and resuspended in the original volume of annealing buffer.

Fifty microliters of Dynal Streptavidin beads (Dynal Biotech Inc., Lake Success, N.Y.; M270 or MyOne™ beads at 10 mg/ml) was washed three times with Annealing Buffer containing 0.1% Tween 20 and resuspended in the original volume in Annealing Buffer containing 0.1% Tween 20. Then the Dynal bead mixture was added to the resuspended sepharose beads. The mixture was vortexed and placed in a rotator for 10 minutes at room temperature.

The beads were collected on the bottom of the test tube by centrifugation at 2300 g (500 rpm for Eppendorf Centrifuge 5415D). The beads were resuspended in the original volume of Annealing Buffer containing 0.1% Tween 20. The mixture, in a test tube, was placed in a magnetic separator (Dynal). The beads were washed three times with Annealing Buffer containing 0.1% Tween 20 and resuspended in the original volume in the same buffer. The beads without amplicons were removed by wash steps, as previously described. Only Sepharose beads containing the appropriated DNA fragments were retained.

The magnetic beads were separated from the sepharose beads by addition of 500 μl of 0.125 M NaOH. The mixture was vortexed and the magnetic beads were removed by magnetic separation. The Sepharose beads remaining in solution was transferred to another tube and washed with 400 μl of 50 mM Tris Acetate until the pH was stabilized at 7.6.

### Example 10

### Nucleic Acid Sequencing Using Bead Emulsion PCR

The following experiment was performed to test the efficacy of the bead emulsion PCR. For this protocol, 600,000 Sepharose beads, with an average diameter of 25-35 μm (as supplied my the manufacturer) were covalently attached to capture primers at a ratio of 30-50 million copies per bead. The beads with covalently attached capture primers were mixed with 1.2 million copies of single stranded Adenovirus Library. The library constructs included a sequence that was complimentary to the capture primer on the beads.

The adenovirus library was annealed to the beads using the procedure described in Example 1. Then, the beads were resuspended in complete PCR solution. The PCR Solution and beads were emulsified in 2 volumes of spinning emulsification oil using the same procedure described in Example 2. The emulsified (encapsulated) beads were subjected to amplification by PCR as outlined in Example 3. The emulsion was broken as outlined in Example 4. DNA on beads was rendered single stranded, sequencing primer was annealed using the procedure of Example 5.

Next, 70,000 beads were sequenced simultaneously by pyrophosphate sequencing using a pyrophosphate sequencer from 454 Life Sciences (New Haven, Conn.). Multiple batches of 70,000 beads were sequenced and the data were listed in Table 6, below.

TABLE 6

| Alignment | | | | | | Inferred |
|---|---|---|---|---|---|---|
| Error | Alignments | | | | | Read |
| Tolerance | None | Single | Multiple | Unique | Coverage | Error |
| 0% | 47916 | 1560 | | 1110 | 54.98% | 0.00% |
| 5% | 46026 | 3450 | | 2357 | 83.16% | 1.88% |
| 10% | 43474 | 6001 | 1 | 3742 | 95.64% | 4.36% |

This table shows the results obtained from BLAST analysis comparing the sequences obtained from the pyrophosphate sequencer against Adenovirus sequence. The first column shows the error tolerance used in the BLAST program. The last column shows the real error as determined by direct comparison to the known sequence.

### 13. Bead Emulsion PCR for Double Ended Sequencing

### Example 11

### Template Quality Control

As indicated previously, the success of the Emulsion PCR reaction was found to be related to the quality of the single stranded template species. Accordingly, the quality of the template material was assessed with two separate quality controls before initiating the Emulsion PCR protocol. First, an aliquot of the single-stranded template was run on the 2100 BioAnalyzer (Agilient). An RNA Pico Chip was used to verify that the sample included a heterogeneous population of fragments, ranging in size from approximately 200 to 500 bases. Second, the library was quantitated using the RiboGreen fluorescence assay on a Bio-Tek FL600 plate fluorometer. Samples determined to have DNA concentrations below 5 ng/μl were deemed too dilute for use.

### Example 12

### DNA Capture Bead Synthesis

Packed beads from a 1 mL N-hydroxysuccinimide ester (NHS)-activated Sepharose HP affinity column (Amersham Biosciences, Piscataway, N.J.) were removed from the column. The 30-25 μm size beads were selected by serial passage through 30 and 25 μm pore filter mesh sections (Sefar America, Depew, N.Y., USA). Beads that passed through the first filter, but were retained by the second were collected and activated as described in the product literature (Amersham Pharmacia Protocol # 71700600AP). Two different amine-labeled HEG (hexaethyleneglycol) long capture primers were obtained, corresponding to the 5′ end of the sense and antisense strand of the template to be amplified, (5′-Amine-3 HEG spacers gcttacctgaccgacctctgcctatcccctgttgcgtgtc-3′; SEQ ID NO:1; and 5′-Amine-3 HEG spacers ccattccccagctcgtcttgccatctgttccctccctgtc-3′; SEQ ID NO:2) (IDT Technologies, Coralville, Iowa, USA). The primers were designed to capture of both strands of the amplification products to allow double ended sequencing, i.e., sequencing the first and second strands of the amplification products. The capture primers were dissolved in 20 mM phosphate buffer, pH 8.0, to obtain a final concentration of 1 mM. Three microliters of each primer were bound to the sieved 30-25 μm beads. The beads were then stored in a bead storage buffer (50 mM Tris, 0.02% Tween and 0.02% sodium azide, pH 8). The beads were quantitated with a hemacytometer (Hausser Scientific, Horsham, Pa., USA) and stored at 4° C. until needed.

### Example 13

### PCR Reaction Mix Preparation and Formulation

As with any single molecule amplification technique, contamination of the reactions with foreign or residual amplicon from other experiments could interfere with a sequencing run. To reduce the possibility of contamination, the PCR reaction mix was prepared in a in a UV-treated laminar flow hood located in a PCR clean room. For each 600,000 bead emulsion PCR reaction, the following reagents were mixed in a 1.5 ml tube: 225 μl of reaction mixture (1× Platinum HiFi Buffer (Invitrogen)), 1 mM dNTPs, 2.5 mM MgSO₄ (Invitrogen), 0.1% BSA, 0.01% Tween, 0.003 U/μl thermostable PPi-ase (NEB), 0.125 μM forward primer (5′-gcttacctgaccgacctctg-3′; SEQ ID NO:3) and 0.125 μM reverse primer (5′-ccattccccagctcgtcttg-3′; SEQ ID NO:4) (IDT Technologies, Coralville, Iowa, USA) and 0.2 U/μl Platinum Hi-Fi Taq Polymerase (Invitrogen). Twenty-five microliters of the reaction mixture was removed and stored in an individual 200 μl PCR tube for use as a negative control. Both the reaction mixture and negative controls were stored on ice until needed.

### Example 14

### Binding Template Species to DNA Capture Beads

Successful clonal DNA amplification for sequencing relates to the delivery of a controlled number of template species to each bead. For the experiments described herein below, the typical target template concentration was determined to be 0.5 template copies per capture bead. At this concentration, Poisson distribution dictates that 61% of the beads have no associated template, 30% have one species of template, and 9% have two or more template species. Delivery of excess species can result in the binding and subsequent amplification of a mixed population (2 or more species) on a single bead, preventing the generation of meaningful sequence data. However, delivery of too few species will result in fewer wells containing template (one species per bead), reducing the extent of sequencing coverage. Consequently, it was deemed that the single-stranded library template concentration was important.

Template nucleic acid molecules were annealed to complimentary primers on the DNA capture beads by the following method, conducted in a UV-treated laminar flow hood. Six hundred thousand DNA capture beads suspended in bead storage buffer (see Example 9, above) were transferred to a 200 μl PCR tube. The tube was centrifuged in a benchtop mini centrifuge for 10 seconds, rotated 180°, and spun for an additional 10 seconds to ensure even pellet formation. The supernatant was removed, and the beads were washed with 200 μl of Annealing Buffer (20 mM Tris, pH 7.5 and 5 mM magnesium acetate). The tube was vortexed for 5 seconds to resuspend the beads, and the beads were pelleted as before. All but approximately 10 μl of the supernatant above the beads was removed, and an additional 200 μl of Annealing Buffer was added. The beads were again vortexed for 5 seconds, allowed to sit for 1 minute, and then pelleted as before. All but 10 μl of supernatant was discarded.

Next, 1.5 μl of 300,000 molecules/μl template library was added to the beads. The tube was vortexed for 5 seconds to mix the contents, and the templates were annealed to the beads in a controlled denaturation/annealing program preformed in an MJ thermocycler. The program allowed incubation for 5 minutes at 80° C., followed by a decrease by 0.1° C./sec to 70° C., incubation for 1 minute at 70° C., decrease by 0.1° C./sec to 60° C., hold at 60° C. for 1 minute, decrease by 0.1° C./sec to 50° C., hold at 50° C. for 1 minute, decrease by 0.1° C./sec to 20° C., hold at 20° C. Following completion of the annealing process, the beads were removed from the thermocycler, centrifuged as before, and the Annealing Buffer was carefully decanted. The capture beads included on average 0.5 copy of single stranded template DNA bound to each bead, and were stored on ice until needed.

### Example 15

### Emulsification

The emulsification process creates a heat-stable water-in-oil emulsion containing 10,000 discrete PCR microreactors per microliter. This serves as a matrix for single molecule, clonal amplification of the individual molecules of the target library. The reaction mixture and DNA capture beads for a single reaction were emulsified in the following manner. In a UV-treated laminar flow hood, 200 μl of PCR solution (from Example 10) was added to the tube containing the 600,000 DNA capture beads (from Example 11). The beads were resuspended through repeated pipetting. After this, the PCR-bead mixture was incubated at room temperature for at least 2 minutes, allowing the beads to equilibrate with the PCR solution. At the same time, 450 μl of Emulsion Oil (4.5% (w:w) Span 80, 1% (w:w) Atlox 4912 (Uniqema, Del.) in light mineral oil (Sigma)) was aliquotted into a flat-topped 2 ml centrifuge tube (Dot Scientific) containing a sterile ¼ inch magnetic stir bar (Fischer). This tube was then placed in a custom-made plastic tube holding jig, which was then centered on a Fisher Isotemp digital stirring hotplate (Fisher Scientific) set to 450 RPM.

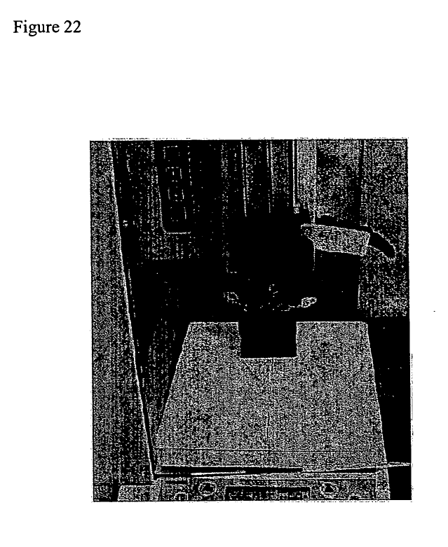

The PCR-bead solution was vortexed for 15 seconds to resuspend the beads. The solution was then drawn into a 1 ml disposable plastic syringe (Benton-Dickenson) affixed with a plastic safety syringe needle (Henry Schein). The syringe was placed into a syringe pump (Cole-Parmer) modified with an aluminum base unit orienting the pump vertically rather than horizontally (FIG. 22). The tube with the emulsion oil was aligned on the stir plate so that it was centered below the plastic syringe needle and the magnetic stir bar was spinning properly. The syringe pump was set to dispense 0.6 ml at 5.5 ml/hr. The PCR-bead solution was added to the emulsion oil in a dropwise fashion. Care was taken to ensure that the droplets did not contact the side of the tube as they fell into the spinning oil.

Once the emulsion was formed, great care was taken to minimize agitation of the emulsion during both the emulsification process and the post-emulsification aliquotting steps. It was found that vortexing, rapid pipetting, or excessive mixing could cause the emulsion to break, destroying the discrete microreactors. In forming the emulsion, the two solutions turned into a homogeneous milky white mixture with the viscosity of mayonnaise. The contents of the syringe were emptied into the spinning oil. Then, the emulsion tube was removed from the holding jig, and gently flicked with a forefinger until any residual oil layer at the top of the emulsion disappeared. The tube was replaced in the holding jig, and stirred with the magnetic stir bar for an additional minute. The stir bar was removed from the emulsion by running a magnetic retrieval tool along the outside of the tube, and the stir bar was discarded.

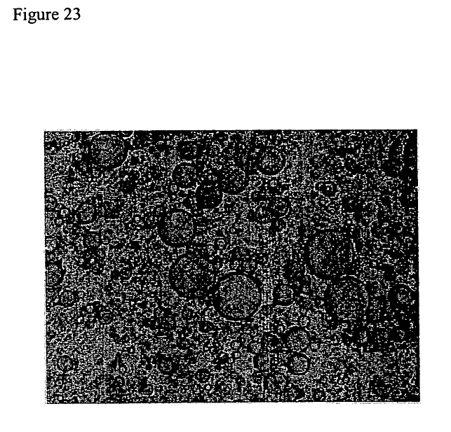

Twenty microliters of the emulsion was taken from the middle of the tube using a P100 pipettor and placed on a microscope slide. The larger pipette tips were used to minimize shear forces. The emulsion was inspected at 50× magnification to ensure that it was comprised predominantly of single beads in 30 to 150 micron diameter microreactors of PCR solution in oil (FIG. 23). After visual examination, the emulsions were immediately amplified.

### Example 16

### Amplification

The emulsion was aliquotted into 7-8 separate PCR tubes. Each tube included approximately 75 μl of the emulsion. The tubes were sealed and placed in a MJ thermocycler along with the 25 μl negative control described above. The following cycle times were used: 1 cycle of incubation for 4 minutes at 94° C. (Hotstart Initiation), 30 cycles of incubation for 30 seconds at 94° C., and 150 seconds at 68° C. (Amplification), and 40 cycles of incubation for 30 seconds at 94° C., and 360 seconds at 68° C. (Hybridization and Extension). After completion of the PCR program, the tubes were removed and the emulsions were broken immediately or the reactions were stored at 10° C. for up to 16 hours prior to initiating the breaking process.

### Example 17

### Breaking the Emulsion and Bead Recovery

Following amplification, the emulstifications were examined for breakage (separation of the oil and water phases). Unbroken emulsions were combined into a single 1.5 ml microcentrifuge tube, while the occasional broken emulsion was discarded. As the emulsion samples were quite viscous, significant amounts remained in each PCR tube. The emulsion remaining in the tubes was recovered by adding 75 μl of mineral oil into each PCR tube and pipetting the mixture. This mixture was added to the 1.5 ml tube containing the bulk of the emulsified material. The 1.5 ml tube was then vortexed for 30 seconds. After this, the tube was centrifuged for 20 minutes in the benchtop microcentrifuge at 13.2K rpm (full speed).

After centrifugation, the emulsion separated into two phases with a large white interface. The clear, upper oil phase was discarded, while the cloudy interface material was left in the tube. In a chemical fume hood, 1 ml hexanes was added to the lower phase and interface layer. The mixture was vortexed for 1 minute and centrifuged at full speed for 1 minute in a benchtop microcentrifuge. The top, oil/hexane phase was removed and discarded. After this, 1 ml of 80% Ethanol/1× Annealing Buffer was added to the remaining aqueous phase, interface, and beads. This mixture was vortexed for 1 minute or until the white material from the interface was dissolved. The sample was then centrifuged in a benchtop microcentrifuge for 1 minute at full speed. The tube was rotated 180 degrees, and spun again for an additional minute. The supernatant was then carefully removed without disturbing the bead pellet.

The white bead pellet was washed twice with 1 ml Annealing Buffer containing 0.1% Tween 20. The wash solution was discarded and the beads were pelleted after each wash as described above. The pellet was washed with 1 ml Picopure water. The beads were pelleted with the centrifuge-rotate-centrifuge method used previously. The aqueous phase was carefully removed. The beads were then washed with 1 ml of 1 mM EDTA as before, except that the beads were briefly vortexed at a medium setting for 2 seconds prior to pelleting and supernatant removal.

Amplified DNA, immobilized on the capture beads, was treated to obtain single stranded DNA. The second strand was removed by incubation in a basic melt solution. One ml of Melt Solution (0.125 M NaOH, 0.2 M NaCl) was subsequently added to the beads. The pellet was resuspended by vortexing at a medium setting for 2 seconds, and the tube placed in a Thermolyne LabQuake tube roller for 3 minutes. The beads were then pelleted as above, and the supernatant was carefully removed and discarded. The residual Melt solution was neutralized by the addition of 1 ml Annealing Buffer. After this, the beads were vortexed at medium speed for 2 seconds. The beads were pelleted, and the supernatant was removed as before. The Annealing Buffer wash was repeated, except that only 800 μl of the Annealing Buffer was removed after centrifugation. The beads and remaining Annealing Buffer were transferred to a 0.2 ml PCR tube. The beads were used immediately or stored at 4° C. for up to 48 hours before continuing on to the enrichment process.

### Example 18

### Optional Bead Enrichment

The bead mass included beads with amplified, immobilized DNA strands, and empty or null beads. As mentioned previously, it was calculated that 61% of the beads lacked template DNA during the amplification process. Enrichment was used to selectively isolate beads with template DNA, thereby maximizing sequencing efficiency. The enrichment process is described in detail below.

The single stranded beads from Example 14 were pelleted with the centrifuge-rotate-centrifuge method, and as much supernatant as possible was removed without disturbing the beads. Fifteen microliters of Annealing Buffer were added to the beads, followed by 2 μl of 100 μM biotinylated, 40 base enrichment primer (5′-Biotin-tetra-ethyleneglycol spacers ccattccccagctcgtcttgccatctgttccctccctgtctcag-3′; SEQ ID NO:5). The primer was complimentary to the combined amplification and sequencing sites (each 20 bases in length) on the 3′ end of the bead-immobilized template. The solution was mixed by vortexing at a medium setting for 2 seconds, and the enrichment primers were annealed to the immobilized DNA strands using a controlled denaturation/annealing program in an MJ thermocycler. The program consisted of the following cycle times and temperatures: incubation for 30 seconds at 65° C., decrease by 0.1° C./sec to 58° C., incubation for 90 seconds at 58° C., and hold at 10° C.

While the primers were annealing, Dynal MyOne™ streptavidin beads were resuspend by gentle swirling. Next, 20 μl of the MyOne™ beads were added to a 1.5 ml microcentrifuge tube containing 1 ml of Enhancing fluid (2 M NaCl, 10 mM Tris-HCl, 1 mM EDTA, pH 7.5). The MyOne bead mixture was vortexed for 5 seconds, and the tube was placed in a Dynal MPC-S magnet. The paramagnetic beads were pelleted against the side of the microcentrifuge tube. The supernatant was carefully removed and discarded without disturbing the MyOne™ beads. The tube was removed from the magnet, and 100 μl of enhancing fluid was added. The tube was vortexed for 3 seconds to resuspend the beads, and stored on ice until needed.

Upon completion of the annealing program, 100 μl of annealing buffer was added to the PCR tube containing the DNA capture beads and enrichment primer. The tube vortexed for 5 seconds, and the contents were transferred to a fresh 1.5 ml microcentrifuge tube. The PCR tube in which the enrichment primer was annealed to the capture beads was washed once with 200 μl of annealing buffer, and the wash solution was added to the 1.5 ml tube. The beads were washed three times with 1 ml of annealing buffer, vortexed for 2 seconds, and pelleted as before. The supernatant was carefully removed. After the third wash, the beads were washed twice with 1 ml of ice cold Enhancing fluid. The beads were vortexed, pelleted, and the supernatant was removed as before. The beads were resuspended in 150 μl ice cold Enhancing fluid and the bead solution was added to the washed MyOne™ beads.

The bead mixture was vortexed for 3 seconds and incubated at room temperature for 3 minutes on a LabQuake tube roller. The streptavidin-coated MyOne™ beads were bound to the biotinylated enrichment primers annealed to immobilized templates on the DNA capture beads. The beads were then centrifuged at 2,000 RPM for 3 minutes, after which the beads were vortexed with 2 second pulses until resuspended. The resuspended beads were placed on ice for 5 minutes. Following this, 500 μl of cold Enhancing fluid was added to the beads and the tube was inserted into a Dynal MPC-S magnet. The beads were left undisturbed for 60 seconds to allow pelleting against the magnet. After this, the supernatant with excess MyOne™ and null DNA capture beads was carefully removed and discarded.

The tube was removed from the MPC-S magnet, and 1 ml of cold enhancing fluid added to the beads. The beads were resuspended with gentle finger flicking. It was important not to vortex the beads at this time, as forceful mixing could break the link between the MyOne™ and DNA capture beads. The beads were returned to the magnet, and the supernatant removed. This wash was repeated three additional times to ensure removal of all null capture beads. To remove the annealed enrichment primers and MyOne™ beads, the DNA capture beads were resuspended in 400 μl of melting solution, vortexed for 5 seconds, and pelleted with the magnet. The supernatant with the enriched beads was transferred to a separate 1.5 ml microcentrifuge tube. For maximum recovery of the enriched beads, a second 400 μl aliquot of melting solution was added to the tube containing the MyOne™ beads. The beads were vortexed and pelleted as before. The supernatant from the second wash was removed and combined with the first bolus of enriched beads. The tube of spent MyOne™ beads was discarded.

The microcentrifuge tube of enriched DNA capture beads was placed on the Dynal MPC-S magnet to pellet any residual MyOne™ beads. The enriched beads in the supernatant were transferred to a second 1.5 ml microcentrifuge tube and centrifuged. The supernatant was removed, and the beads were washed 3 times with 1 ml of annealing buffer to neutralize the residual melting solution. After the third wash, 800 μl of the supernatant was removed, and the remaining beads and solution were transferred to a 0.2 ml PCR tube. The enriched beads were centrifuged at 2,000 RPM for 3 minutes and the supernatant decanted. Next, 20 μl of annealing buffer and 3 μl of two different 100 μM sequencing primers (5′-ccatctgttccctccctgtc-3′; SEQ ID NO:6; and 5′-cctatcccctgttgcgtgtc-3′ phosphate; SEQ ID NO:7) were added. The tube was vortexed for 5 seconds, and placed in an MJ thermocycler for the following 4-stage annealing program: incubation for 5 minutes at 65° C., decrease by 0.1° C./sec to 50° C., incubation for 1 minute at 50° C., decrease by 0.1° C./sec to 40° C., hold at 40° C. for 1 minute, decrease by 0.1° C. to 15° C., and hold at 15° C.

Upon completion of the annealing program, the beads were removed from thermocycler and pelleted by centrifugation for 10 seconds. The tube was rotated 180°, and spun for an additional 10 seconds. The supernatant was decanted and discarded, and 200 μl of annealing buffer was added to the tube. The beads were resuspended with a 5 second vortex, and pelleted as before. The supernatant was removed, and the beads resuspended in 100 μl annealing buffer. At this point, the beads were quantitated with a Multisizer 3 Coulter Counter (Beckman Coulter). Beads were stored at 4° C. and were stable for at least 1 week.

### Example 19

### Double Strand Sequencing

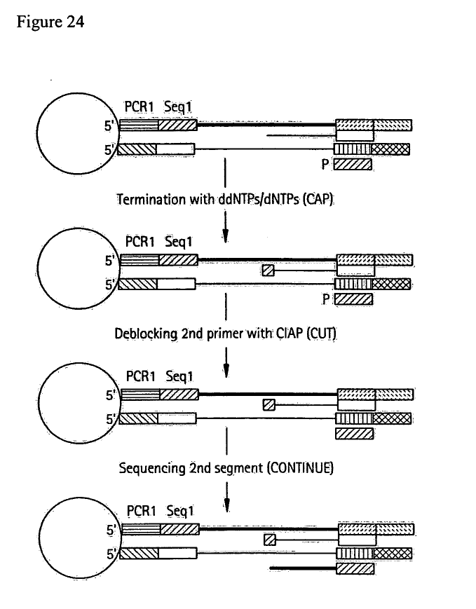

For double strand sequencing, two different sequencing primers are used; an unmodified primer MMP7A and a 3′ phosphorylated primer MMP2Bp. There are multiple steps in the process. This process is shown schematically in FIG. 24.
1. First Strand Sequencing. Sequencing of the first strand involves extension of the unmodified primer by a DNA polymerase through sequential addition of nucleotides for a predetermined number of cycles.
2. CAPPING: The first strand sequencing was terminated by flowing a Capping Buffer containing 25 mM Tricine, 5 mM Mangesium acetate, 1 mM DTT, 0.4 mg/ml PVP, 0.1 mg/ml BSA, 0.01% Tween and 2 μM of each dideoxynucleotides and 2 μM of each deoxynucleotide.
3. CLEAN: The residual deoxynucleotides and dideoxynucleotides was removed by flowing in Apyrase Buffer containing 25 mM Tricine, 5 mM Magnesium acetate, 1 mM DTT, 0.4 mg/ml PVP, 0.1 mg/ml BSA, 0.01% Tween and 8.5 units/L of Apyrase.
4. CUTTING: The second blocked primer was unblocked by removing the phosphate group from the 3′ end of the modified 3′ phosphorylated primer by flowing a Cutting buffer containing 5 units/ml of Calf intestinal phosphatases.
5. CONTINUE: The second unblocked primer was activated by addition of polymerase by flowing 1000 units/ml of DNA polymerases to capture all the available primer sites.
6. Second Strand Sequencing: Sequencing of the second strand by a DNA polymerase through sequential addition of nucleotides for a predetermined number of cycles.

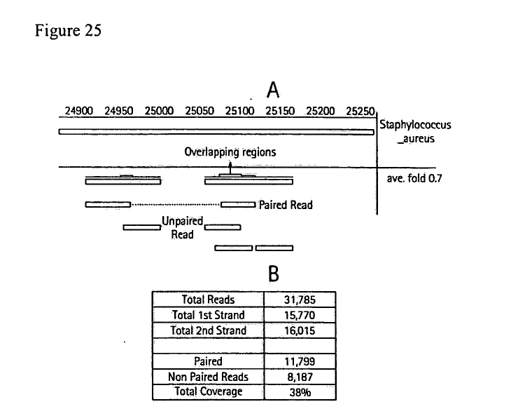

Using the methods described above, the genomic DNA of Staphylococcus aureus was sequenced. The results are presented in FIG. 25. A total of 31,785 reads were obtained based on 15770 reads of the first strand and 16015 reads of the second strand. Of these, a total of 11,799 reads were paired and 8187 reads were unpaired obtaining a total coverage of 38%.

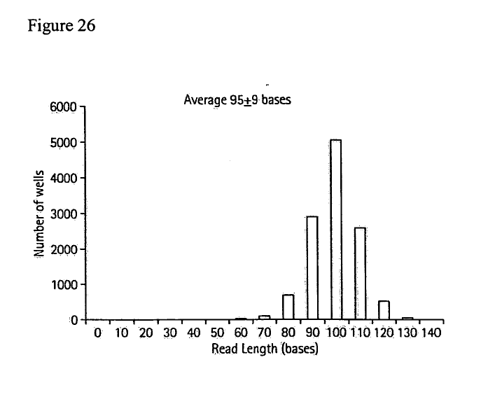

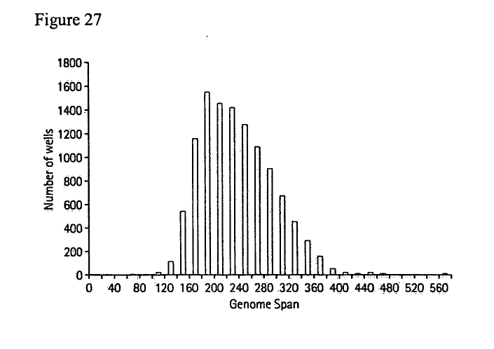

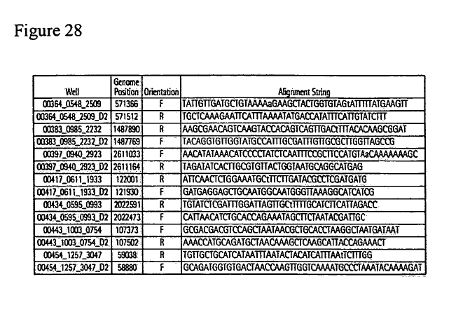

Read lengths ranged from 60 to 130 with an average of 95±9 bases (FIG. 26). The distribution of genome span and the number of wells of each genome span is shown in FIG. 27. Representative alignment strings, from this genomic sequencing, are shown in FIG. 28.

### Example 20

### Template PCR

30 micron NHS Sepharose beads were coupled with 1 mM of each of the following primers:

| | |
|---|---|
| MMP1A: cgtttcccctgtgtgccttg | (SEQ ID NO:8) |
| MMP1B: ccatctgttgcgtgcgtgtc | (SEQ ID NO:9) |

Drive-to-bead PCR was performed in a tube on the MJ thermocycler by adding 50 μl of washed primer-coupled beads to a PCR master mix at a one-to-one volume-to-volume ratio. The PCR master mixture included:
1× PCR buffer;
1 mM of each dNTP;
0.625 μM primer MMP1A;
0.625 μM primer MMP1B;
1 μl of 1 unit/μl Hi Fi Taq (Invitrogen, San Diego, Calif.); and
˜5-10 ng Template DNA (the DNA to be sequenced).

The PCR reaction was performed by programming the MJ thermocycler for the following: incubation at 94° C. for 3 minutes; 39 cycles of incubation at 94° C. for 30 seconds, 58° C. for 30 seconds, 68° C. for 30 seconds; followed by incubation at 94° C. for 30 seconds and 58° C. for 10 minutes; 10 cycles of incubation at 94° C. for 30 seconds, 58° C. for 30 seconds, 68° C. for 30 seconds; and storage at 10° C.

### Example 21

### Template DNA Preparation and Annealing Sequencing Primer

The beads from Example 1 were washed two times with distilled water; washed once with 1 mM EDTA, and incubated with 0.125 M NaOH for 5 minutes. This removed the DNA strands not linked to the beads. Then, the beads were washed once with 50 mM Tris Acetate buffer, and twice with Annealing Buffer: 200 mM Tris-Acetate, 50 mM Mg Acetate, pH 7.5. Next, 500 pmoles of Sequencing Primer MMP7A (ccatctgttccctccctgtc; SEQ ID NO:10) and MMP2B-phos (cctatcccctgttgcgtgtc; SEQ ID NO:11) were added to the beads. The primers were annealed with the following program on the MJ thermocycler: incubation at 60° C. for 5 minutes; temperature drop of 0.1 degree per second to 50° C.; incubation at 50° C. for 5 minutes; temperature drop of 0.1 degree per second to 4° C.; incubation at 40° C. for 5 minutes; temperature drop of 0.1 degree per second to 10° C. The template was then sequenced using standard pyrophosphate sequencing.

### Example 22

### Sequencing and Stopping of the First Strand

The beads were spun into a 55 μm PicoTiter plate (PTP) at 3000 rpm for 10 minutes. The PTP was placed on a rig and run using de novo sequencing for a predetermined number of cycles. The sequencing was stopped by capping the first strand. The first strand was capped by adding 100 μl of 1× AB (50 mM Mg Acetate, 250 mM Tricine), 1000 unit/ml BST polymerase, 0.4 mg/ml single strand DNA binding protein, 1 mM DTT, 0.4 mg/ml PVP (Polyvinyl Pyrolidone), 10 uM of each ddNTP, and 2.5 μM of each dNTP. Apyrase was then flowed over in order to remove excess nucleotides by adding 1× AB, 0.4 mg/ml PVP, 1 mM DTT, 0.1 mg/ml BSA, 0.125 units/ml apyrase, incubated for 20 minutes.

### Example 23

### Preparation of Second Strand for Sequencing

The second strand was unblocked by adding 100 μl of 1× AB, 0.1 unit per ml poly nucleotide kinase, 5 mM DTT. The resultant template was sequenced using standard pyrophosphate sequencing (described, e.g., in U.S. Pat. Nos. 6,274,320, 6258,568 and 6,210,891, incorporated herein by reference). The results of the sequencing method can be seen in FIG. 21F where a fragment of 174 bp was sequenced on both ends using pyrophosphate sequencing and the methods described in these examples.

### REFERENCES

1. Vogelstein, B. & Kinzler, K. W. (2002) The genetic basis of human cancer (McGraw-Hill Health Professions Division, New York).

2. Scriver, C. R., Beaudet, A. L., Sly W. S., Valle, D. (2001) The metabolic and molecular bases of inherited disease (McGraw-Hill Health Professions Division, New York).

3. Kallioniemi, A., Kallioniemi, O. P., Sudar, D., Rutovitz, D., Gray, J. W., Waldman, F. & Pinkel, D. (1992) Science 258, 818-21.

4. Lisitsyn, N., Lisitsyn, N. & Wigler, M. (1993) Science 259, 946-51.

5. Schrock, E., du Manoir, S., Veldman, T., Schoell, B., Wienberg, J., Ferguson-Smith, M. A., Ning, Y., Ledbetter, D. H., Bar-Am, I., Soenksen, D., Garini, Y. & Ried, T. (1996) Science 273, 494-7.

6. Speicher, M. R., Gwyn Ballard, S. & Ward, D. C. (1996) Nat Genet 12, 368-75.

7. Solinas-Toldo, S., Lampel, S., Stilgenbauer, S., Nickolenko, J., Benner, A., Dohner, H., Cremer, T. & Lichter, P. (1997) Genes Chromosomes Cancer 20, 399-407.

8. Pinkel, D., Segraves, R., Sudar, D., Clark, S., Poole, I., Kowbel, D., Collins, C., Kuo, W. L., Chen, C., Zhai, Y., Dairkee, S. H., Ljung, B. M., Gray, J. W. & Albertson, D. G. (1998) Nat Genet 20, 207-11.

9. Pollack, J. R., Perou, C. M., Alizadeh, A. A., Eisen, M. B., Pergamenschikov, A., Williams, C. F., Jeffrey, S. S., Botstein, D. & Brown, P. O. (1999) Nat Genet 23, 41-6.

10. Cai, W. W., Mao, J. H., Chow, C. W., Damani, S., Balmain, A. & Bradley, A. (2002) Nat Biotechnol 20, 393-6.

11. Knuutila, S., Bjorkqvist, A. M., Autio, K., Tarkkanen, M., Wolf, M., Monni, O., Szymanska, J., Larramendy, M. L., Tapper, J., Pere, H., El-Rifai, W., Hemmer, S., Wasenius, V. M., Vidgren, V. & Zhu, Y. (1998) Am J Pathol 152, 1107-23.

12. Knuutila, S., Aalto, Y., Autio, K., Bjorkqvist, A. M., El-Rifai, W., Hemmer, S., Huhta, T., Kettunen, E., Kiuru-Kuhlefelt, S., Larramendy, M. L., Lushnikova, T., Monni, O., Pere, H., Tapper, J., Tarkkanen, M., Varis, A., Wasenius, V. M., Wolf, M. & Zhu, Y. (1999) Am J Pathol 155, 683-94.

13. Carpenter, N. J. (2001) Semin Pediatr Neurol 8, 135-46.

14. Hodgson, G., Hager, J. H., Volik, S., Hariono, S., Wernick, M., Moore, D., Nowak, N., Albertson, D. G., Pinkel, D., Collins, C., Hanahan, D. & Gray, J. W. (2001) Nat Genet 29, 459-64.

15. Gray, J. W. & Collins, C. (2000) Carcinogenesis 21, 443-52.

16. Snijders, A. M., Nowak, N., Segraves, R., Blackwood, S., Brown, N., Conroy, J., Hamilton, G., Hindle, A. K., Huey, B., Kimura, K., Law, S., Myambo, K., Palmer, J., Ylstra, B., Yue, J. P., Gray, J. W., Jain, A. N., Pinkel, D. & Albertson, D. G. (2001) Nat Genet 29, 263-4.

17. Wang T L, Maierhofer C, Speicher M R, Lengauer C, Vogelstein B, Kinzler K W, Velculescu V E. (2002) Proc Natl Acad Sci USA. 99(25):16156-61.

18. Sebat J, Lakshmi B, Troge J, Alexander J, Young J, Lundin P, Maner S, Massa H, Walker M, Chi M, Navin N, Lucito R, Healy J, Hicks J, Ye K, Reiner A, Gilliam T C, Trask B, Patterson N, Zetterberg A, Wigler M. (2004) Science. 305(5683):525-528.

19. Hamilton, S. C., J. W. Farchaus and M. C. Davis. 2001. DNA polymerases as engines for biotechnology. BioTechniques 31:370.

20. QiaQuick Spin Handbook (QIAGEN, 2001): hypertext transfer protocol://world wide web.qiagen.com/literature/handbooks/qqspin/1016893HBQQSpin_PCR_mc_prot.pdf.

21. Quick Ligation Kit (NEB): hypertext transfer protocol://world wide web.neb.com/neb/products/mod_enzymes/M2200.html.

22. MinElute kit (QIAGEN): hypertext transfer protocol://world wide web.qiagen.com/literature/handbooks/minelute/1016839_HBMinElute_Prot_Gel.pdf.

23. Biomagnetic Techniques in Molecular Biology, Technical Handbook, 3rd edition (Dynal, 1998): hypertext transfer protocol://world wide web.dynal.no/kunder/dynal/DynalPub36.nsf/cb927fbab 127a0ad4125683b004b011c/4908f5b 1a665858a41256adf05779f2/$FILE/Dynabeads M-280 Streptavidin.pdf.

24. Bio Analyzer User Manual (Agilent): hypertext transfer protocol://world wide web.chem.agilent.com/temp/rad31B29/00033620.pdf

All patents and publications cited in this specification are hereby incorporated by reference herein, including the previous disclosure provided by U.S. application Ser. No. 60/513,319 filed Oct. 23, 2003.

1. A method of karyotyping a genome of a test cell, comprising:
a) obtaining a plurality of test DNA sequences from random locations of the genome of the test cell;
b) mapping said test DNA sequences to a genomic scaffold to obtain a test distribution of mapped sequences to a test region;
c) comparing the test distribution to a reference distribution of obtained from a reference cell;
d) identifying a statistically significant alteration between the test distribution and the reference distribution
wherein if present said alteration indicates a karyotypic difference between the test cell and the reference cell.

2. The method of claim 1, wherein the test and reference distribution are within a contiguous region in the genome.

3. The method of claim 1, wherein the reference distribution comprises a database.

4. The method of claim 3, wherein the database comprises the mapped sequences from a reference genome.

5. The method of claim 1, further comprising prior to step(c):
1) obtaining a plurality of reference DNA sequences from random locations of a reference genome of a reference cell and
2) mapping said reference DNA sequences to a genomic scaffold to obtain a reference distribution of reference sequences to a reference region of the genomic scaffold to generate a reference distribution of mapped sequences.

6. The method of claim 1 wherein said statistically significant alteration is at confidence level of a p-value of less than 0.05.

7. The method of claim 1 wherein said statistically significant alteration is at confidence level of a p-value of less than 0.01.

8. The method of claim 1 wherein said statistically significant alteration is at confidence level of a p-value of less than 0.001.

9. The method of claim 1 wherein said statistically significant alteration is at confidence level of a p-value of less than 1/24.

10. The method of claim 1 wherein said statistically significant alteration is at confidence level of a p-value of less than 1/23.

11. The method of claim 1 wherein said statistically significant alteration is at confidence level of a p-value of less than 1/22.

12. The method of claim 1 wherein the test cell and the reference cell are of the same species.

13. The method of claim 1 wherein said test cell is a eukaryotic cell.

14. The method of claim 13, wherein said eukaryotic cell is a human cell.

15. The method of claim 14, wherein said eukaryotic cell is a cancer cell.

16. The method of claim 1 wherein the test cell is a cell from a subject with a hereditary disorder.

17. The method of claim 13, wherein said eukaryotic cell is isolated from amniotic fluid.

18. The method of claim 13, wherein said eukaryotic cell is from an embryo, or a fetus.

19. The method of claim 18, wherein said embryo is derived from in vitro fertilization.

20. The method of claim 1, wherein the test and the reference distribution of mapped sequences comprises more than 1000 mapped sequences.

21. The method of claim 1, wherein the test and the reference distribution of mapped sequences comprises more than 10,000 mapped sequences.

22. The method of claim 1, wherein the test and the reference distribution of mapped sequences comprises more than 100,000 mapped sequences.

23. The method of claim 1, wherein the test region comprises a single chromosome.

24. The method of claim 1, wherein the test region comprise two or more chromosomes.

25. The method of claim 2, wherein the contiguous region is about 4 Mb in length.

26. The method of claim 2, wherein the contiguous region is about 2 Mb in length.

27. The method of claim 2, wherein the contiguous region is 500 kb in length.

28. The method of claim 2, wherein the contiguous region is about 250 kb in length.

29. The method of claim 2, wherein the contiguous region is about 60 kb in length.

30. The method of claim 2, wherein the contiguous region is about 30 kb in length.

31. The method of claim 2, wherein the contiguous region is about 10 kb in length.

32. The method of claim 2, wherein said plurality of test DNA sequences are obtained by:
a) providing DNA from a test cell;
b) randomly fragmenting said DNA into a plurality of DNA fragments; and
c) determining the sequence of at least 20 bases from each said DNA fragments.

33. The method of claim 32, wherein the fragmenting is by an enzyme.

34. The method of or claim 33, wherein the enzyme is DNAase 1.

35. The method of claim 32, wherein the fragmenting is by a mechanical method.

36. The method of claim 35, wherein the mechanical method is sonication or nebulization.

37. The method of claim 1, wherein said plurality of DNA fragment comprises at least 1000 DNA fragments.

38. The method of claim 1, wherein said plurality of DNA fragment comprises at least 10,000 DNA fragments.

39. The method of claim 1, wherein said plurality of DNA fragment comprises at least 100,000 DNA fragments.

40. The method of claim 1, wherein said plurality of DNA fragment comprises at least 1,000,000 DNA fragments.

41. The method of claim 1, wherein the mapping step is performed by recording the location and number of occurrences of each of the plurality of DNA sequences.

42. The method of claim 1, wherein a test distribution/reference distribution ratio greater than 1.5 or less than 0.75 is indicative of aneuploidy.

43. The method of claim 1, wherein said test region and reference region is in a sex chromosome, wherein said reference region is from a male cell and said test region is in a female cell, and a test distribution/reference distribution ratio greater than 3.0 or less than 1.5 is indicative of aneuploidy.

44. The method of claim 1, wherein said test region and reference region is in a sex chromosome, wherein said reference region is from a female cell and said test region is in a male cell, and a test distribution/reference distribution ratio greater than 3.0 or less than 1.5 is indicative of aneuploidy.
